SEQUENCE LISTING (619 sequences)

SEQ ID NO: 1 - protein, 28 aa, Artificial Sequence
  synthetic construct; ApoE mimetic
LRKLRKRLLRDWLKAFYDKVAEKLKEAF

SEQ ID NO: 2 - protein, 28 aa, Artificial Sequence
  synthetic construct; reverse analog of ApoE mimetic
RLLRKRLKRLDWLKAFYDKVAEKLKEAF

SEQ ID NO: 3 - protein, 28 aa, Artificial Sequence
  synthetic construct; reverse analog of ApoE mimetic
LRKLRKRLLRFAEKLKEAVKDYFAKLWD

SEQ ID NO: 4 - protein, 10 aa, Artificial Sequence
  synthetic construct; receptor binding domain of human Apo E
LRKLRKRLLR

SEQ ID NO: 5 - protein, 18 aa, Artificial Sequence
  synthetic construct; lipid-associating peptide
DWLKAFYDKVAEKLKEAF

SEQ ID NO: 6 - protein, 10 aa, Artificial Sequence
  synthetic construct; analog of receptor binding domain of human Apo E
LRRLRRRLLR

SEQ ID NO: 7 - protein, 10 aa, Artificial Sequence
  synthetic construct; Rat ApoE mimetic
LRKMRKRLMR

SEQ ID NO: 8 - protein, 10 aa, Artificial Sequence
  synthetic construct; Bovine Apo E mimetic
LRKLPKRLLR

SEQ ID NO: 9 - protein, 10 aa, Artificial Sequence
  synthetic construct; Pig ApoE mimetic
LRNVRKRLVR

SEQ ID NO: 10 - protein, 10 aa, Artificial Sequence
  synthetic construct; Dog ApoE mimetic
MRKLRKRVLR

SEQ ID NO: 11 - protein, 10 aa, Artificial Sequence
  synthetic construct; R modified ApoE mimetic
LRRLRRRLLR

SEQ ID NO: 12 - protein, 10 aa, Artificial Sequence
  synthetic construct; F modified ApoE mimetic
LRKLRKRFFR

SEQ ID NO: 13 - protein, 9 aa, Artificial Sequence
  synthetic construct; ApoB mimetic
RLTRKRGLK

SEQ ID NO: 14 - protein, 18 aa, Artificial Sequence
  synthetic construct; class A peptide
DWFKAFYDKVAEKLKEAF

SEQ ID NO: 15 - protein, 18 aa, Artificial Sequence
  synthetic construct; class A peptide
DWLKAFYDKVAEKFKEAF

SEQ ID NO: 16 - protein, 18 aa, Artificial Sequence
  synthetic construct; class A peptide
DWFKAFYDKVAEKFKEAF

SEQ ID NO: 17 - protein, 18 aa, Artificial Sequence
  synthetic construct; class A peptide
DWLKAFYDKVFEKFKEFF

SEQ ID NO: 18 - protein, 18 aa, Artificial Sequence
  synthetic construct; class A peptide
DWLKAFYDKFFEKFKEFF

SEQ ID NO: 19 - protein, 18 aa, Artificial Sequence
  synthetic construct; class A peptide
DWFKAFYDKFFEKFKEFF

SEQ ID NO: 20 - protein, 18 aa, Artificial Sequence
  synthetic construct; class A peptide
DWLKAFYDKVAEKLKEFF

SEQ ID NO: 21 - protein, 18 aa, Artificial Sequence
  synthetic construct; class A peptide
DWLKAFYDKVFEKLKEFF

SEQ ID NO: 22 - protein, 18 aa, Artificial Sequence
  synthetic construct; class A peptide
DWLKAFYDKVFEKFKEFF

SEQ ID NO: 23 - protein, 18 aa, Artificial Sequence
  synthetic construct; class A peptide
EWLKLFYEKVLEKFKEAF

SEQ ID NO: 24 - protein, 19 aa, Artificial Sequence
  synthetic construct; class A peptide
EWLKAFYIDKVAEKFKEAF

SEQ ID NO: 25 - protein, 19 aa, Artificial Sequence
  synthetic construct; class A peptide
EEWLKAFYDKVAEKLKEFF

SEQ ID NO: 26 - protein, 18 aa, Artificial Sequence
  synthetic construct; class A peptide
EWLKAFYDKVFEKFKEAF

SEQ ID NO: 27 - protein, 18 aa, Artificial Sequence
  synthetic construct; class A peptide
EWLKAFYDKVFEKLKEFF

SEQ ID NO: 28 - protein, 18 aa, Artificial Sequence
  synthetic construct; class A peptide
EWLKAFYDKVAEKFKEFF

SEQ ID NO: 29 - protein, 18 aa, Artificial Sequence
  synthetic construct; class A peptide
EWLKAFYDKVFEKFKEFF

SEQ ID NO: 30 - protein, 14 aa, Artificial Sequence
  synthetic construct; class A peptide
AFYDKVAEKLKEAF

SEQ ID NO: 31 - protein, 14 aa, Artificial Sequence
  synthetic construct; class A peptide
AFYDKVAEKFKEAF

SEQ ID NO: 32 - protein, 14 aa, Artificial Sequence
  synthetic construct; class A peptide
AFYDKVAEKFKEAF

SEQ ID NO: 33 - protein, 14 aa, Artificial Sequence
  synthetic construct; class A peptide
AFYDKFFEKFKEFF

SEQ ID NO: 34 - protein, 14 aa, Artificial Sequence
  synthetic construct; class A peptide
AFYDKFFEKFKEFF

SEQ ID NO: 35 - protein, 14 aa, Artificial Sequence
  synthetic construct; class A peptide
AFYDKVAEKFKEAF

SEQ ID NO: 36 - protein, 14 aa, Artificial Sequence
  synthetic construct; class A peptide
AFYDKVAEKLKEFF

SEQ ID NO: 37 - protein, 14 aa, Artificial Sequence
  synthetic construct; class A peptide
AFYDKVFEKFKEAF

SEQ ID NO: 38 - protein, 14 aa, Artificial Sequence
  synthetic construct; class A peptide
AFYDKVFEKLKEFF

SEQ ID NO: 39 - protein, 14 aa, Artificial Sequence
  synthetic construct; class A peptide
AFYDKVAEKFKEFF

SEQ ID NO: 40 - protein, 14 aa, Artificial Sequence
  synthetic construct; class A peptide
KAFYDKVFEKFKEF

SEQ ID NO: 41 - protein, 14 aa, Artificial Sequence
  synthetic construct; class A peptide
LFYEKVLEKFKEAF

SEQ ID NO: 42 - protein, 14 aa, Artificial Sequence
  synthetic construct; class A peptide
AFYDKVAEKFKEAF

SEQ ID NO: 43 - protein, 14 aa, Artificial Sequence
  synthetic construct; class A peptide
AFYDKVAEKLKEFF

SEQ ID NO: 44 - protein, 14 aa, Artificial Sequence
  synthetic construct; class A peptide
AFYDKVFEKFKEAF

SEQ ID NO: 45 - protein, 14 aa, Artificial Sequence
  synthetic construct; class A peptide
AFYDKVFEKLKEFF

SEQ ID NO: 46 - protein, 14 aa, Artificial Sequence
  synthetic construct; class A peptide
AFYDKVAEKFKEFF

SEQ ID NO: 47 - protein, 14 aa, Artificial Sequence
  synthetic construct; class A peptide
AFYDKVFEKFKEFF

SEQ ID NO: 48 - protein, 18 aa, Artificial Sequence
  synthetic construct; class A peptide
DWLKALYDKVAEKLKEAL

SEQ ID NO: 49 - protein, 18 aa, Artificial Sequence
  synthetic construct; class A peptide
DWFKAFYEKVAEKLKEFF

SEQ ID NO: 50 - protein, 18 aa, Artificial Sequence
  synthetic construct; class A peptide
DWFKAFYEKFFEKFKEFF

SEQ ID NO: 51 - protein, 18 aa, Artificial Sequence
  synthetic construct; class A peptide
EWLKALYEKVAEKLKEAL

SEQ ID NO: 52 - protein, 18 aa, Artificial Sequence
  synthetic construct; class A peptide
EWLKAFYEKVAEKLKEAF

SEQ ID NO: 53 - protein, 18 aa, Artificial Sequence
  synthetic construct; class A peptide
EWFKAFYEKVAEKLKEFF

SEQ ID NO: 54 - protein, 18 aa, Artificial Sequence
  synthetic construct; class A peptide
EWLKAFYEKVFEKFKEFF

SEQ ID NO: 55 - protein, 18 aa, Artificial Sequence
  synthetic construct; class A peptide
EWLKAFYEKFFEKFKEFF

SEQ ID NO: 56 - protein, 18 aa, Artificial Sequence
  synthetic construct; class A peptide
EWFKAFYEKFFEKFKEFF

SEQ ID NO: 57 - protein, 18 aa, Artificial Sequence
  synthetic construct; class A peptide
DFLKAWYDKVAEKLKEAW

SEQ ID NO: 58 - protein, 18 aa, Artificial Sequence
  synthetic construct; class A peptide
EFLKAWYEKVAEKLKEAW

SEQ ID NO: 59 - protein, 18 aa, Artificial Sequence
  synthetic construct; class A peptide
DFWKAWYDKVAEKLKEWW

SEQ ID NO: 60 - protein, 18 aa, Artificial Sequence
  synthetic construct; class A peptide
EFWKAWYEKVAEKLKEWW

SEQ ID NO: 61 - protein, 18 aa, Artificial Sequence
  synthetic construct; class A peptide
DKLKAFYDKVFEWAKEAF

SEQ ID NO: 62 - protein, 18 aa, Artificial Sequence
  synthetic construct; class A peptide
DKWKAVYDKFAEAFKEFL

SEQ ID NO: 63 - protein, 18 aa, Artificial Sequence
  synthetic construct; class A peptide
EKLKAFYEKVFEWAKEAF

SEQ ID NO: 64 - protein, 18 aa, Artificial Sequence
  synthetic construct; class A peptide
EKWKAVYEKFAEAFKEFL

SEQ ID NO: 65 - protein, 18 aa, Artificial Sequence
  synthetic construct; class A peptide
DWLKAFVDKFAEKFKEAY

SEQ ID NO: 66 - protein, 18 aa, Artificial Sequence
  synthetic construct; class A peptide
EKWKAVYEKFAEAFKEFL

SEQ ID NO: 67 - protein, 18 aa, Artificial Sequence
  synthetic construct; class A peptide
DWLKAFVYDKVFKLKEFF

SEQ ID NO: 68 - protein, 18 aa, Artificial Sequence
  synthetic construct; class A peptide
EWLKAFVYEKVFKLKEFF

SEQ ID NO: 69 - protein, 18 aa, Artificial Sequence
  synthetic construct; class A peptide
DWLRAFYDKVAEKLKEAF

SEQ ID NO: 70 - protein, 18 aa, Artificial Sequence
  synthetic construct; class A peptide
EWLRAFYEKVAEKLKEAF

SEQ ID NO: 71 - protein, 18 aa, Artificial Sequence
  synthetic construct; class A peptide
DWLKAFYDRVAEKLKEAF

SEQ ID NO: 72 - protein, 18 aa, Artificial Sequence
  synthetic construct; class A peptide
EWLKAFYERVAEKLKEAF

SEQ ID NO: 73 - protein, 18 aa, Artificial Sequence
  synthetic construct; class A peptide
DWLKAFYDKVAERLKEAF

SEQ ID NO: 74 - protein, 18 aa, Artificial Sequence
  synthetic construct; class A peptide
EWLKAFYEKVAERLKEAF

SEQ ID NO: 75 - protein, 18 aa, Artificial Sequence
  synthetic construct; class A peptide
DWLKAFYDKVAEKLREAF

SEQ ID NO: 76 - protein, 18 aa, Artificial Sequence
  synthetic construct; class A peptide
EWLKAFYEKVAEKLREAF

SEQ ID NO: 77 - protein, 18 aa, Artificial Sequence
  synthetic construct; class A peptide
DWLKAFYDRVAERLKEAF

SEQ ID NO: 78 - protein, 18 aa, Artificial Sequence
  synthetic construct; class A peptide
EWLKAFYERVAERLKEAF

SEQ ID NO: 79 - protein, 18 aa, Artificial Sequence
  synthetic construct; class A peptide
DWLRAFYDKVAEKLREAF

SEQ ID NO: 80 - protein, 18 aa, Artificial Sequence
  synthetic construct; class A peptide
EWLRAFYEKVAEKLREAF

SEQ ID NO: 81 - protein, 18 aa, Artificial Sequence
  synthetic construct; class A peptide
DWLRAFYDRVAEKLKEAF

SEQ ID NO: 82 - protein, 18 aa, Artificial Sequence
  synthetic construct; class A peptide
EWLRAFYERVAEKLKEAF

SEQ ID NO: 83 - protein, 18 aa, Artificial Sequence
  synthetic construct; class A peptide
DWLKAFYDKVAERLREAF

SEQ ID NO: 84 - protein, 18 aa, Artificial Sequence
  synthetic construct; class A peptide
EWLKAFYEKVAERLREAF

SEQ ID NO: 85 - protein, 18 aa, Artificial Sequence
  synthetic construct; class A peptide
DWLRAFYDKVAERLKEAF

SEQ ID NO: 86 - protein, 18 aa, Artificial Sequence
  synthetic construct; class A peptide
EWLRAFYEKVAERLKEAF

SEQ ID NO: 87 - protein, 37 aa, Artificial Sequence
  synthetic construct; class A peptide
DWLKAFYDKVAEKLKEAFPDWLKAFYDKVAEKLKEAF

SEQ ID NO: 88 - protein, 37 aa, Artificial Sequence
  synthetic construct; class A peptide
DWLKAFYDKVAEKLKEFFPDWLKAFYDKVAEKLKEFF

SEQ ID NO: 89 - protein, 37 aa, Artificial Sequence
  synthetic construct; class A peptide
DWFKAFYDKVAEKLKEAFPDWFKAFYDKVAEKLKEAF

SEQ ID NO: 90 - protein, 37 aa, Artificial Sequence
  synthetic construct; class A peptide
DKLKAFYDKVFEWAKEAFPDKLKAFYDKVFEWLKEAF

SEQ ID NO: 91 - protein, 37 aa, Artificial Sequence
  synthetic construct; class A peptide
DKWKAVYDKFAEAFKEFLPDKWKAVYDKFAEAFKEFL

SEQ ID NO: 92 - protein, 37 aa, Artificial Sequence
  synthetic construct; class A peptide
DWFKAFYDKVAEKFKEAFPDWFKAFYDKVAEKFKEAF

SEQ ID NO: 93 - protein, 37 aa, Artificial Sequence
  synthetic construct; class A peptide
DWLKAFVYDKVFKLKEFFPDWLKAFVYDKVFKLKEFF

SEQ ID NO: 94 - protein, 37 aa, Artificial Sequence
  synthetic construct; class A peptide
DWLKAFYDKFAEKFKEFFPDWLKAFYDKFAEKFKEFF

SEQ ID NO: 95 - protein, 18 aa, Artificial Sequence
  synthetic construct; class A peptide
EWFKAFYEKVAEKFKEAF

SEQ ID NO: 96 - protein, 14 aa, Artificial Sequence
  synthetic construct; class A peptide
DWFKAFYDKVAEKF

SEQ ID NO: 97 - protein, 14 aa, Artificial Sequence
  synthetic construct; class A peptide
FKAFYDKVAEKFKE

SEQ ID NO: 98 - protein, 14 aa, Artificial Sequence
  synthetic construct; class A peptide
FKAFYEKVAEKFKE

SEQ ID NO: 99 - protein, 14 aa, Artificial Sequence
  synthetic construct; class A peptide
FKAFYDKVAEKFKE

SEQ ID NO: 100 - protein, 14 aa, Artificial Sequence
  synthetic construct; class A peptide
FKAFYEKVAEKFKE

SEQ ID NO: 101 - protein, 18 aa, Artificial Sequence
  synthetic construct; class A peptide
DWFKAFYDKVAEKFKEAF

SEQ ID NO: 102 - protein, 18 aa, Artificial Sequence
  synthetic construct; class A peptide
EWFKAFYEKVAEKFKEAF

SEQ ID NO: 103 - protein, 14 aa, Artificial Sequence
  synthetic construct; class A peptide
AFYDKVAEKFKEAF

SEQ ID NO: 104 - protein, 14 aa, Artificial Sequence
  synthetic construct; class A peptide
DWFKAFYDKVAEKF

SEQ ID NO: 105 - protein, 18 aa, Artificial Sequence
  synthetic construct; class A peptide
DWLKAFYDKVFEKFKEFF

SEQ ID NO: 106 - protein, 18 aa, Artificial Sequence
  synthetic construct; class A peptide
DWLKAFYDKVFEKFKEFF

SEQ ID NO: 107 - protein, 18 aa, Artificial Sequence
  synthetic construct; class A peptide
EWLKAFYEKVFEKFKEFF

SEQ ID NO: 108 - protein, 18 aa, Artificial Sequence
  synthetic construct; class A peptide
EWLKAFYEKVFEKFKEFF

SEQ ID NO: 109 - protein, 14 aa, Artificial Sequence
  synthetic construct; class A peptide
AFYDKVFEKFKEFF

SEQ ID NO: 110 - protein, 14 aa, Artificial Sequence
  synthetic construct; class A peptide
AFYDKVFEKFKEFF

SEQ ID NO: 111 - protein, 14 aa, Artificial Sequence
  synthetic construct; class A peptide
AFYEKVFEKFKEFF

SEQ ID NO: 112 - protein, 14 aa, Artificial Sequence
  synthetic construct; class A peptide
AFYEKVFEKFKEFF

SEQ ID NO: 113 - protein, 14 aa, Artificial Sequence
  synthetic construct; class A peptide
DWLKAFYDKVFEKF

SEQ ID NO: 114 - protein, 14 aa, Artificial Sequence
  synthetic construct; class A peptide
DWLKAFYDKVFEKF

SEQ ID NO: 115 - protein, 14 aa, Artificial Sequence
  synthetic construct; class A peptide
EWLKAFYEKVFEKF

SEQ ID NO: 116 - protein, 14 aa, Artificial Sequence
  synthetic construct; class A peptide
EWLKAFYEKVFEKF

SEQ ID NO: 117 - protein, 14 aa, Artificial Sequence
  synthetic construct; class A peptide
LKAFYDKVFEKFKE

SEQ ID NO: 118 - protein, 14 aa, Artificial Sequence
  synthetic construct; class A peptide
LKAFYDKVFEKFKE

SEQ ID NO: 119 - protein, 14 aa, Artificial Sequence
  synthetic construct; class A peptide
LKAFYEKVFEKFKE

SEQ ID NO: 120 - protein, 14 aa, Artificial Sequence
  synthetic construct; class A peptide
LKAFYEKVFEKFKE

SEQ ID NO: 121 - protein, 18 aa, Artificial Sequence
  synthetic construct; modified class A peptide
DKWKAVYDKFAEAFKEFL

SEQ ID NO: 122 - protein, 18 aa, Artificial Sequence
  synthetic construct; modified class A peptide
DKLKAFYDKVFEWAKEAF

SEQ ID NO: 123 - protein, 18 aa, Artificial Sequence
  synthetic construct; Class A amphipathic helical peptides
EWFKAFYEKVADKFKDAF

SEQ ID NO: 124 - protein, 18 aa, Artificial Sequence
  synthetic construct; Class A amphipathic helical peptides
EWFKAFYDKVADKFKEAF

SEQ ID NO: 125 - protein, 18 aa, Artificial Sequence
  synthetic construct; Class A amphipathic helical peptides
DWFKAFYEKVADKFKEAF

SEQ ID NO: 126 - protein, 18 aa, Artificial Sequence
  synthetic construct; Class A amphipathic helical peptides
DWFKAFYEKVAEKFKDAF

SEQ ID NO: 127 - protein, 18 aa, Artificial Sequence
  synthetic construct; Class A amphipathic helical peptides
DFWKAFYDKVAEKFKEAF

SEQ ID NO: 128 - protein, 18 aa, Artificial Sequence
  synthetic construct; Class A amphipathic helical peptides
EFWKAFYEKVADKFKDAF

SEQ ID NO: 129 - protein, 18 aa, Artificial Sequence
  synthetic construct; Class A amphipathic helical peptides
EFWKAFYDKVADKFKEAF

SEQ ID NO: 130 - protein, 18 aa, Artificial Sequence
  synthetic construct; Class A amphipathic helical peptides
DFWKAFYEKVADKFKEAF

SEQ ID NO: 131 - protein, 18 aa, Artificial Sequence
  synthetic construct; Class A amphipathic helical peptides
DFWKAFYEKVAEKFKDAF

SEQ ID NO: 132 - protein, 18 aa, Artificial Sequence
  synthetic construct; Class A amphipathic helical peptides
DWFKAYFDKVAEKFKEAF

SEQ ID NO: 133 - protein, 18 aa, Artificial Sequence
  synthetic construct; Class A amphipathic helical peptides
EWFKAYFEKVADKFKDAF

SEQ ID NO: 134 - protein, 18 aa, Artificial Sequence
  synthetic construct; Class A amphipathic helical peptides
EWFKAYFDKVADKFKEAF

SEQ ID NO: 135 - protein, 18 aa, Artificial Sequence
  synthetic construct; Class A amphipathic helical peptides
DWFKAYFEKVADKFKEAF

SEQ ID NO: 136 - protein, 18 aa, Artificial Sequence
  synthetic construct; Class A amphipathic helical peptides
DWFKAYFEKVAEKFKDAF

SEQ ID NO: 137 - protein, 18 aa, Artificial Sequence
  synthetic construct; Class A amphipathic helical peptides
DWFKAFVDKYAEKFKEAF

SEQ ID NO: 138 - protein, 18 aa, Artificial Sequence
  synthetic construct; Class A amphipathic helical peptides
EWFKAFVEKYADKFKDAF

SEQ ID NO: 139 - protein, 18 aa, Artificial Sequence
  synthetic construct; Class A amphipathic helical peptides
EWFKAFVDKYADKFKEAF

SEQ ID NO: 140 - protein, 18 aa, Artificial Sequence
  synthetic construct; Class A amphipathic helical peptides
DWFKAFVEKYADKFKEAF

SEQ ID NO: 141 - protein, 18 aa, Artificial Sequence
  synthetic construct; Class A amphipathic helical peptides
DWFKAFVEKYAEKFKDAF

SEQ ID NO: 142 - protein, 18 aa, Artificial Sequence
  synthetic construct; Class A amphipathic helical peptides
DWFKAFYDKAVEKFKEAF

SEQ ID NO: 143 - protein, 18 aa, Artificial Sequence
  synthetic construct; Class A amphipathic helical peptides
EWFKAFYEKAVDKFKDAF

SEQ ID NO: 144 - protein, 18 aa, Artificial Sequence
  synthetic construct; Class A amphipathic helical peptides
EWFKAFYDKAVDKFKEAF

SEQ ID NO: 145 - protein, 18 aa, Artificial Sequence
  synthetic construct; Class A amphipathic helical peptides
DWFKAFYEKAVDKFKEAF

SEQ ID NO: 146 - protein, 18 aa, Artificial Sequence
  synthetic construct; Class A amphipathic helical peptides
DWFKAFYEKAVEKFKDAF

SEQ ID NO: 147 - protein, 18 aa, Artificial Sequence
  synthetic construct; Class A amphipathic helical peptides
DWFKAFYDKVFEKAKEAF

SEQ ID NO: 148 - protein, 18 aa, Artificial Sequence
  synthetic construct; Class A amphipathic helical peptides
EWFKAFYEKVFDKAKDAF

SEQ ID NO: 149 - protein, 18 aa, Artificial Sequence
  synthetic construct; Class A amphipathic helical peptides
EWFKAFYDKVFDKAKEAF

SEQ ID NO: 150 - protein, 18 aa, Artificial Sequence
  synthetic construct; Class A amphipathic helical peptides
DWFKAFYEKVFDKAKEAF

SEQ ID NO: 151 - protein, 18 aa, Artificial Sequence
  synthetic construct; Class A amphipathic helical peptides
DWFKAFYEKVFEKAKDAF

SEQ ID NO: 152 - protein, 18 aa, Artificial Sequence
  synthetic construct; Class A amphipathic helical peptides
DWFKAFYDKVAEKAKEFF

SEQ ID NO: 153 - protein, 18 aa, Artificial Sequence
  synthetic construct; Class A amphipathic helical peptides
EWFKAFYEKVADKAKDFF

SEQ ID NO: 154 - protein, 18 aa, Artificial Sequence
  synthetic construct; Class A amphipathic helical peptides
EWFKAFYDKVADKAKEFF

SEQ ID NO: 155 - protein, 18 aa, Artificial Sequence
  synthetic construct; Class A amphipathic helical peptides
DWFKAFYEKVADKAKEFF

SEQ ID NO: 156 - protein, 18 aa, Artificial Sequence
  synthetic construct; Class A amphipathic helical peptides
DWFKAFYEKVAEKAKDFF

SEQ ID NO: 157 - protein, 18 aa, Artificial Sequence
  synthetic construct; Class A amphipathic helical peptides
DWFKAFYDKVAEKFKEFA

SEQ ID NO: 158 - protein, 18 aa, Artificial Sequence
  synthetic construct; Class A amphipathic helical peptides
EWFKAFYEKVADKFKDFA

SEQ ID NO: 159 - protein, 18 aa, Artificial Sequence
  synthetic construct; Class A amphipathic helical peptides
EWFKAFYDKVADKFKEFA

SEQ ID NO: 160 - protein, 18 aa, Artificial Sequence
  synthetic construct; Class A amphipathic helical peptides
DWFKAFYEKVADKFKEFA

SEQ ID NO: 161 - protein, 18 aa, Artificial Sequence
  synthetic construct; Class A amphipathic helical peptides
DWFKAFYEKVAEKFKDFA

SEQ ID NO: 162 - protein, 18 aa, Artificial Sequence
  synthetic construct; Class A amphipathic helical peptides
DAFKAFYDKVAEKFKEWF

SEQ ID NO: 163 - protein, 18 aa, Artificial Sequence
  synthetic construct; Class A amphipathic helical peptides
EAFKAFYEKVADKFKDWF

SEQ ID NO: 164 - protein, 18 aa, Artificial Sequence
  synthetic construct; Class A amphipathic helical peptides
EAFKAFYDKVADKFKEWF

SEQ ID NO: 165 - protein, 18 aa, Artificial Sequence
  synthetic construct; Class A amphipathic helical peptides
DAFKAFYEKVADKFKEWF

SEQ ID NO: 166 - protein, 18 aa, Artificial Sequence
  synthetic construct; Class A amphipathic helical peptides
DAFKAFYEKVAEKFKDWF

SEQ ID NO: 167 - protein, 18 aa, Artificial Sequence
  synthetic construct; Class A amphipathic helical peptides
DAFKAFYDKVWEKFKEAF

SEQ ID NO: 168 - protein, 18 aa, Artificial Sequence
  synthetic construct; Class A amphipathic helical peptides
EAFKAFYEKVWDKFKDAF

SEQ ID NO: 169 - protein, 18 aa, Artificial Sequence
  synthetic construct; Class A amphipathic helical peptides
EAFKAFYDKVWDKFKEAF

SEQ ID NO: 170 - protein, 18 aa, Artificial Sequence
  synthetic construct; Class A amphipathic helical peptides
DAFKAFYEKVWDKFKEAF

SEQ ID NO: 171 - protein, 18 aa, Artificial Sequence
  synthetic construct; Class A amphipathic helical peptides
DAFKAFYEKVWEKFKDAF

SEQ ID NO: 172 - protein, 18 aa, Artificial Sequence
  synthetic construct; Class A amphipathic helical peptides
DYFKAFWDKVAEKFKEAF

SEQ ID NO: 173 - protein, 18 aa, Artificial Sequence
  synthetic construct; Class A amphipathic helical peptides
EYFKAFWEKVADKFKDAF

SEQ ID NO: 174 - protein, 18 aa, Artificial Sequence
  synthetic construct; Class A amphipathic helical peptides
EYFKAFWDKVADKFKEAF

SEQ ID NO: 175 - protein, 18 aa, Artificial Sequence
  synthetic construct; Class A amphipathic helical peptides
DYFKAFWEKVADKFKEAF

SEQ ID NO: 176 - protein, 18 aa, Artificial Sequence
  synthetic construct; Class A amphipathic helical peptides
DYFKAFWEKVAEKFKDAF

SEQ ID NO: 177 - protein, 18 aa, Artificial Sequence
  synthetic construct; Class A amphipathic helical peptides
DWAKAFYDKVAEKFKEFF

SEQ ID NO: 178 - protein, 18 aa, Artificial Sequence
  synthetic construct; Class A amphipathic helical peptides
EWAKAFYEKVADKFKDFF

SEQ ID NO: 179 - protein, 18 aa, Artificial Sequence
  synthetic construct; Class A amphipathic helical peptides
EWAKAFYDKVADKFKEFF

SEQ ID NO: 180 - protein, 18 aa, Artificial Sequence
  synthetic construct; Class A amphipathic helical peptides
DWAKAFYEKVADKFKEFF

SEQ ID NO: 181 - protein, 18 aa, Artificial Sequence
  synthetic construct; Class A amphipathic helical peptides
DWAKAFYEKVAEKFKDFF

SEQ ID NO: 182 - protein, 18 aa, Artificial Sequence
  synthetic construct; Class A amphipathic helical peptides
DWFKAAYDKVAEKFKEFF

SEQ ID NO: 183 - protein, 18 aa, Artificial Sequence
  synthetic construct; Class A amphipathic helical peptides
EWFKAAYEKVADKFKDFF

SEQ ID NO: 184 - protein, 18 aa, Artificial Sequence
  synthetic construct; Class A amphipathic helical peptides
EWFKAAYDKVADKFKEFF

SEQ ID NO: 185 - protein, 18 aa, Artificial Sequence
  synthetic construct; Class A amphipathic helical peptides
DWFKAAYEKVADKFKEFF

SEQ ID NO: 186 - protein, 18 aa, Artificial Sequence
  synthetic construct; Class A amphipathic helical peptides
DWFKAAYEKVAEKFKDFF

SEQ ID NO: 187 - protein, 18 aa, Artificial Sequence
  synthetic construct; Class A amphipathic helical peptides
DWFKAFADKVAEKFKEYF

SEQ ID NO: 188 - protein, 18 aa, Artificial Sequence
  synthetic construct; Class A amphipathic helical peptides
EWFKAFAEKVADKFKDYF

SEQ ID NO: 189 - protein, 18 aa, Artificial Sequence
  synthetic construct; Class A amphipathic helical peptides
EWFKAFADKVADKFKEYF

SEQ ID NO: 190 - protein, 18 aa, Artificial Sequence
  synthetic construct; Class A amphipathic helical peptides
DWFKAFAEKVADKFKEYF

SEQ ID NO: 191 - protein, 18 aa, Artificial Sequence
  synthetic construct; Class A amphipathic helical peptides
DWFKAFAEKVAEKFKDYF

SEQ ID NO: 192 - protein, 18 aa, Artificial Sequence
  synthetic construct; Class A amphipathic helical peptides
DWFKAFYDKAAEKFKEVF

SEQ ID NO: 193 - protein, 18 aa, Artificial Sequence
  synthetic construct; Class A amphipathic helical peptides
EWFKAFYEKAADKFKDVF

SEQ ID NO: 194 - protein, 18 aa, Artificial Sequence
  synthetic construct; Class A amphipathic helical peptides
EWFKAFYDKAADKFKEVF

SEQ ID NO: 195 - protein, 18 aa, Artificial Sequence
  synthetic construct; Class A amphipathic helical peptides
DWFKAFYEKAADKFKEVF

SEQ ID NO: 196 - protein, 18 aa, Artificial Sequence
  synthetic construct; Class A amphipathic helical peptides
DWFKAFYEKAAEKFKDVF

SEQ ID NO: 197 - protein, 18 aa, Artificial Sequence
  synthetic construct; Class A amphipathic helical peptides
DWYKAFFDKVAEKFKEAF

SEQ ID NO: 198 - protein, 18 aa, Artificial Sequence
  synthetic construct; Class A amphipathic helical peptides
EWYKAFFEKVADKFKDAF

SEQ ID NO: 199 - protein, 18 aa, Artificial Sequence
  synthetic construct; Class A amphipathic helical peptides
EWYKAFFDKVADKFKEAF

SEQ ID NO: 200 - protein, 18 aa, Artificial Sequence
  synthetic construct; Class A amphipathic helical peptides
DWYKAFFEKVADKFKEAF

SEQ ID NO: 201 - protein, 18 aa, Artificial Sequence
  synthetic construct; Class A amphipathic helical peptides
DWYKAFFEKVAEKFKDAF

SEQ ID NO: 202 - protein, 18 aa, Artificial Sequence
  synthetic construct; Class A amphipathic helical peptides
DWVKAFYDKFAEKFKEAF

SEQ ID NO: 203 - protein, 18 aa, Artificial Sequence
  synthetic construct; Class A amphipathic helical peptides
EWVKAFYEKFADKFKDAF

SEQ ID NO: 204 - protein, 18 aa, Artificial Sequence
  synthetic construct; Class A amphipathic helical peptides
EWVKAFYDKFADKFKEAF

SEQ ID NO: 205 - protein, 18 aa, Artificial Sequence
  synthetic construct; Class A amphipathic helical peptides
DWVKAFYEKFADKFKEAF

SEQ ID NO: 206 - protein, 18 aa, Artificial Sequence
  synthetic construct; Class A amphipathic helical peptides
DWVKAFYEKFAEKFKDAF

SEQ ID NO: 207 - protein, 18 aa, Artificial Sequence
  synthetic construct; Class A amphipathic helical peptides
DWFKAFFDKVAEKYKEAF

SEQ ID NO: 208 - protein, 18 aa, Artificial Sequence
  synthetic construct; Class A amphipathic helical peptides
EWFKAFFEKVADKYKDAF

SEQ ID NO: 209 - protein, 18 aa, Artificial Sequence
  synthetic construct; Class A amphipathic helical peptides
EWFKAFFDKVADKYKEAF

SEQ ID NO: 210 - protein, 18 aa, Artificial Sequence
  synthetic construct; Class A amphipathic helical peptides
DWFKAFFEKVADKYKEAF

SEQ ID NO: 211 - protein, 18 aa, Artificial Sequence
  synthetic construct; Class A amphipathic helical peptides
DWFKAFFEKVADKYKEAF

SEQ ID NO: 212 - protein, 18 aa, Artificial Sequence
  synthetic construct; Class A amphipathic helical peptides
DWFKAFFDKVAEKFKEAY

SEQ ID NO: 213 - protein, 18 aa, Artificial Sequence
  synthetic construct; Class A amphipathic helical peptides
EWFKAFFEKVADKFKDAY

SEQ ID NO: 214 - protein, 18 aa, Artificial Sequence
  synthetic construct; Class A amphipathic helical peptides
EWFKAFFDKVADKFKEAY

SEQ ID NO: 215 - protein, 18 aa, Artificial Sequence
  synthetic construct; Class A amphipathic helical peptides
DWFKAFFEKVADKFKEAY

SEQ ID NO: 216 - protein, 18 aa, Artificial Sequence
  synthetic construct; Class A amphipathic helical peptides
DWFKAFFEKVAEKFKDAY

SEQ ID NO: 217 - protein, 18 aa, Artificial Sequence
  synthetic construct; Class A amphipathic helical peptides
DWFKAFYDKFAEKFKEAV

SEQ ID NO: 218 - protein, 18 aa, Artificial Sequence
  synthetic construct; Class A amphipathic helical peptides
EWFKAFYEKFADKFKDAV

SEQ ID NO: 219 - protein, 18 aa, Artificial Sequence
  synthetic construct; Class A amphipathic helical peptides
EWFKAFYDKFADKFKEAV

SEQ ID NO: 220 - protein, 18 aa, Artificial Sequence
  synthetic construct; Class A amphipathic helical peptides
DWFKAFYEKFADKFKEAV

SEQ ID NO: 221 - protein, 18 aa, Artificial Sequence
  synthetic construct; Class A amphipathic helical peptides
DWFKAFYEKFAEKFKDAV

SEQ ID NO: 222 - protein, 18 aa, Artificial Sequence
  synthetic construct; Class A amphipathic helical peptides
DKFKAFYDKVAEKFWEAF

SEQ ID NO: 223 - protein, 18 aa, Artificial Sequence
  synthetic construct; Class A amphipathic helical peptides
EKFKAFYEKVADKFWDAF

SEQ ID NO: 224 - protein, 18 aa, Artificial Sequence
  synthetic construct; Class A amphipathic helical peptides
EKFKAFYDKVADKFWEAF

SEQ ID NO: 225 - protein, 18 aa, Artificial Sequence
  synthetic construct; Class A amphipathic helical peptides
DKFKAFYEKVADKFWEAF

SEQ ID NO: 226 - protein, 18 aa, Artificial Sequence
  synthetic construct; Class A amphipathic helical peptides
DKFKAFYEKVAEKFWDAF

SEQ ID NO: 227 - protein, 18 aa, Artificial Sequence
  synthetic construct; Class A amphipathic helical peptides
DKWKAFYDKVAEKFFEAF

SEQ ID NO: 228 - protein, 18 aa, Artificial Sequence
  synthetic construct; Class A amphipathic helical peptides
EKWKAFYEKVADKFFDAF

SEQ ID NO: 229 - protein, 18 aa, Artificial Sequence
  synthetic construct; Class A amphipathic helical peptides
EKWKAFYDKVADKFFEAF

SEQ ID NO: 230 - protein, 18 aa, Artificial Sequence
  synthetic construct; Class A amphipathic helical peptides
DKWKAFYEKVADKFFEAF

SEQ ID NO: 231 - protein, 18 aa, Artificial Sequence
  synthetic construct; Class A amphipathic helical peptides
DKWKAFYEKVAEKFFDAF

SEQ ID NO: 232 - protein, 18 aa, Artificial Sequence
  synthetic construct; Class A amphipathic helical peptides
DKFKAFYDKWAEVFKEAF

SEQ ID NO: 233 - protein, 18 aa, Artificial Sequence
  synthetic construct; Class A amphipathic helical peptides
EKFKAFYEKWADVFKDAF

SEQ ID NO: 234 - protein, 18 aa, Artificial Sequence
  synthetic construct; Class A amphipathic helical peptides
EKFKAFYDKWADVFKEAF

SEQ ID NO: 235 - protein, 18 aa, Artificial Sequence
  synthetic construct; Class A amphipathic helical peptides
DKFKAFYEKWADVFKEAF

SEQ ID NO: 236 - protein, 18 aa, Artificial Sequence
  synthetic construct; Class A amphipathic helical peptides
DKFKAFYEKWAEVFKDAF

SEQ ID NO: 237 - protein, 18 aa, Artificial Sequence
  synthetic construct; Class A amphipathic helical peptides
DKFKAFYDKVAEFWKEAF

SEQ ID NO: 238 - protein, 18 aa, Artificial Sequence
  synthetic construct; Class A amphipathic helical peptides
EKFKAFYEKVADFWKDAF

SEQ ID NO: 239 - protein, 18 aa, Artificial Sequence
  synthetic construct; Class A amphipathic helical peptides
EKFCAFYDKVADFWKEAF

SEQ ID NO: 240 - protein, 18 aa, Artificial Sequence
  synthetic construct; Class A amphipathic helical peptides
DKFKAFYEKVADFWKEAF

SEQ ID NO: 241 - protein, 18 aa, Artificial Sequence
  synthetic construct; Class A amphipathic helical peptides
DKFKAFYEKVAEFWKDAF

SEQ ID NO: 242 - protein, 18 aa, Artificial Sequence
  synthetic construct; Class A amphipathic helical peptides
FAEKFKEAVKDYFAKFWD

SEQ ID NO: 243 - protein, 18 aa, Artificial Sequence
  synthetic construct; Class A amphipathic helical peptides
FADKFKDAVKEYFAKFWE

SEQ ID NO: 244 - protein, 18 aa, Artificial Sequence
  synthetic construct; Class A amphipathic helical peptides
FADKFKEAVKDYFAKFWE

SEQ ID NO: 245 - protein, 18 aa, Artificial Sequence
  synthetic construct; Class A amphipathic helical peptides
FAEKFKDAVKEYFAKFWD

SEQ ID NO: 246 - protein, 18 aa, Artificial Sequence
  synthetic construct; Class A amphipathic helical peptides
FAEKFKDAVKDYFAKFWE

SEQ ID NO: 247 - protein, 18 aa, Artificial Sequence
  synthetic construct; Class A amphipathic helical peptides
FWEKFKEAVKDYFAKFAD

SEQ ID NO: 248 - protein, 18 aa, Artificial Sequence
  synthetic construct; Class A amphipathic helical peptides
FWDKFKDAVKEYFAKFAE

SEQ ID NO: 249 - protein, 18 aa, Artificial Sequence
  synthetic construct; Class A amphipathic helical peptides
FADKFKEAVKDYFAKFWE

SEQ ID NO: 250 - protein, 18 aa, Artificial Sequence
  synthetic construct; Class A amphipathic helical peptides
FAEKFKDAVKEYFAKFWD

SEQ ID NO: 251 - protein, 18 aa, Artificial Sequence
  synthetic construct; Class A amphipathic helical peptides
FAEKFKDAVKDYFAKFWE

SEQ ID NO: 252 - protein, 18 aa, Artificial Sequence
  synthetic construct; Class A amphipathic helical peptides
FFEKFKEAVKDYFAKAWD

SEQ ID NO: 253 - protein, 18 aa, Artificial Sequence
  synthetic construct; Class A amphipathic helical peptides
FFDKFKDAVKEYFAKAWE

SEQ ID NO: 254 - protein, 18 aa, Artificial Sequence
  synthetic construct; Class A amphipathic helical peptides
FFDKFKEAVKDYFAKAWE

SEQ ID NO: 255 - protein, 18 aa, Artificial Sequence
  synthetic construct; Class A amphipathic helical peptides
FFEKFKDAVKEYFAKAWD

SEQ ID NO: 256 - protein, 18 aa, Artificial Sequence
  synthetic construct; Class A amphipathic helical peptides
FFEKFKDAVKDYFAKAWE

SEQ ID NO: 257 - protein, 18 aa, Artificial Sequence
  synthetic construct; Class A amphipathic helical peptides
FAEKAKEFVKDYFAKFWD

SEQ ID NO: 258 - protein, 18 aa, Artificial Sequence
  synthetic construct; Class A amphipathic helical peptides
FADKAKDFVKEYFAKFWE

SEQ ID NO: 259 - protein, 18 aa, Artificial Sequence
  synthetic construct; Class A amphipathic helical peptides
FADKAKEFVKDYFAKFWE

SEQ ID NO: 260 - protein, 18 aa, Artificial Sequence
  synthetic construct; Class A amphipathic helical peptides
FAEKAKDFVKEYFAKFWD

SEQ ID NO: 261 - protein, 18 aa, Artificial Sequence
  synthetic construct; Class A amphipathic helical peptides
FAEKAKDFVKDYFAKFWE

SEQ ID NO: 262 - protein, 18 aa, Artificial Sequence
  synthetic construct; Class A amphipathic helical peptides
FAEKFKEVAKDYFAKFWD

SEQ ID NO: 263 - protein, 18 aa, Artificial Sequence
  synthetic construct; Class A amphipathic helical peptides
FADKFKDVAKEYFAKFWE

SEQ ID NO: 264 - protein, 18 aa, Artificial Sequence
  synthetic construct; Class A amphipathic helical peptides
FADKFKEVAKDYFAKFWE

SEQ ID NO: 265 - protein, 18 aa, Artificial Sequence
  synthetic construct; Class A amphipathic helical peptides
FAEKFKDVAKEYFAKFWD

SEQ ID NO: 266 - protein, 18 aa, Artificial Sequence
  synthetic construct; Class A amphipathic helical peptides
FAEKFKDVAKDYFAKFWE

SEQ ID NO: 267 - protein, 18 aa, Artificial Sequence
  synthetic construct; Class A amphipathic helical peptides
FADKFKEAYKDVFAKFWE

SEQ ID NO: 268 - protein, 18 aa, Artificial Sequence
  synthetic construct; Class A amphipathic helical peptides
FAEKFKDAYKEVFAKFWD

SEQ ID NO: 269 - protein, 18 aa, Artificial Sequence
  synthetic construct; Class A amphipathic helical peptides
FAEKFKDAYKDVFAKFWE

SEQ ID NO: 270 - protein, 18 aa, Artificial Sequence
  synthetic construct; Class A amphipathic helical peptides
FAEKFKEAVKDFYAKFWD

SEQ ID NO: 271 - protein, 18 aa, Artificial Sequence
  synthetic construct; Class A amphipathic helical peptides
FADKFKDAVKEFYAKFWE

SEQ ID NO: 272 - protein, 18 aa, Artificial Sequence
  synthetic construct; Class A amphipathic helical peptides
FADKFKEAVKDFYAKFWE

SEQ ID NO: 273 - protein, 18 aa, Artificial Sequence
  synthetic construct; Class A amphipathic helical peptides
FAEKFKDAVKEFYAKFWD

SEQ ID NO: 274 - protein, 18 aa, Artificial Sequence
  synthetic construct; Class A amphipathic helical peptides
FAEKFKDAVKDFYAKFWE

SEQ ID NO: 275 - protein, 18 aa, Artificial Sequence
  synthetic construct; Class A amphipathic helical peptides
FAEKFWEAVKDYFAKFKD

SEQ ID NO: 276 - protein, 18 aa, Artificial Sequence
  synthetic construct; Class A amphipathic helical peptides
FADKFWDAVKEYFAKFKE

SEQ ID NO: 277 - protein, 18 aa, Artificial Sequence
  synthetic construct; Class A amphipathic helical peptides
FADKFWEAVKDYFAKFKE

SEQ ID NO: 278 - protein, 18 aa, Artificial Sequence
  synthetic construct; Class A amphipathic helical peptides
FAEKFWDAVKEYFAKFKD

SEQ ID NO: 279 - protein, 18 aa, Artificial Sequence
  synthetic construct; Class A amphipathic helical peptides
FAEKFWDAVKDYFAKFKE

SEQ ID NO: 280 - protein, 18 aa, Artificial Sequence
  synthetic construct; Class A amphipathic helical peptides
AFEKFKEAVKDYFAKFWD

SEQ ID NO: 281 - protein, 18 aa, Artificial Sequence
  synthetic construct; Class A amphipathic helical peptides
AFDKFKDAVKEYFAKFWE

SEQ ID NO: 282 - protein, 18 aa, Artificial Sequence
  synthetic construct; Class A amphipathic helical peptides
AFDKFKEAVKDYFAKFWE

SEQ ID NO: 283 - protein, 18 aa, Artificial Sequence
  synthetic construct; Class A amphipathic helical peptides
AFEKFKDAVKEYFAKFWD

SEQ ID NO: 284 - protein, 18 aa, Artificial Sequence
  synthetic construct; Class A amphipathic helical peptides
AFEKFKDAVKDYFAKFWE

SEQ ID NO: 285 - protein, 18 aa, Artificial Sequence
  synthetic construct; Class A amphipathic helical peptides
VAEKFKEAFKDYFAKFWD

SEQ ID NO: 286 - protein, 18 aa, Artificial Sequence
  synthetic construct; Class A amphipathic helical peptides
VADKFKDAFKEYFAKFWE

SEQ ID NO: 287 - protein, 18 aa, Artificial Sequence
  synthetic construct; Class A amphipathic helical peptides
VADKFKEAFKDYFAKFWE

SEQ ID NO: 288 - protein, 18 aa, Artificial Sequence
  synthetic construct; Class A amphipathic helical peptides
VAEKFKDAFKEYFAKFWD

SEQ ID NO: 289 - protein, 18 aa, Artificial Sequence
  synthetic construct; Class A amphipathic helical peptides
VAEKFKDAFKDYFAKFWE

SEQ ID NO: 290 - protein, 18 aa, Artificial Sequence
  synthetic construct; Class A amphipathic helical peptides
YAEKFKEAVKDFFAKFWD

SEQ ID NO: 291 - protein, 18 aa, Artificial Sequence
  synthetic construct; Class A amphipathic helical peptides
YADKFKDAVKEFFAKFWE

SEQ ID NO: 292 - protein, 18 aa, Artificial Sequence
  synthetic construct; Class A amphipathic helical peptides
YADKFKEAVKDFFAKFWE

SEQ ID NO: 293 - protein, 18 aa, Artificial Sequence
  synthetic construct; Class A amphipathic helical peptides
YAEKFKDAVKEFFAKFWD

SEQ ID NO: 294 - protein, 18 aa, Artificial Sequence
  synthetic construct; Class A amphipathic helical peptides
YAEKFKDAVKDFFAKFWE

SEQ ID NO: 295 - protein, 18 aa, Artificial Sequence
  synthetic construct; Class A amphipathic helical peptides
AAEKFKEFVKDYFAKFWD

SEQ ID NO: 296 - protein, 18 aa, Artificial Sequence
  synthetic construct; Class A amphipathic helical peptides
AADKFKDFVKEYFAKFWE

SEQ ID NO: 297 - protein, 18 aa, Artificial Sequence
  synthetic construct; Class A amphipathic helical peptides
AADKFKEFVKDYFAKFWE

SEQ ID NO: 298 - protein, 18 aa, Artificial Sequence
  synthetic construct; Class A amphipathic helical peptides
AAEKFKDFVKEYFAKFWD

SEQ ID NO: 299 - protein, 18 aa, Artificial Sequence
  synthetic construct; Class A amphipathic helical peptides
AAEKFKDFVKDYFAKFWE

SEQ ID NO: 300 - protein, 18 aa, Artificial Sequence
  synthetic construct; Class A amphipathic helical peptides
FFEKAKEAVKDYFAKFWD

SEQ ID NO: 301 - protein, 18 aa, Artificial Sequence
  synthetic construct; Class A amphipathic helical peptides
FFDKAKDAVKEYFAKFWE

SEQ ID NO: 302 - protein, 18 aa, Artificial Sequence
  synthetic construct; Class A amphipathic helical peptides
FFDKAKEAVKDYFAKFWE

SEQ ID NO: 303 - protein, 16 aa, Artificial Sequence
  synthetic construct; Class A amphipathic helical peptides
EKAKDAVKEYFAKFWD

SEQ ID NO: 304 - protein, 16 aa, Artificial Sequence
  synthetic construct; Class A amphipathic helical peptides
EKAKDAVKDYFAKFWE

SEQ ID NO: 305 - protein, 18 aa, Artificial Sequence
  synthetic construct; Class A amphipathic helical peptides
FYEKFKEAVKDAFAKFWD

SEQ ID NO: 306 - protein, 18 aa, Artificial Sequence
  synthetic construct; Class A amphipathic helical peptides
FYDKFKDAVKEAFAKFWE

SEQ ID NO: 307 - protein, 18 aa, Artificial Sequence
  synthetic construct; Class A amphipathic helical peptides
FYDKFKEAVKDAFAKFWE

SEQ ID NO: 308 - protein, 18 aa, Artificial Sequence
  synthetic construct; Class A amphipathic helical peptides
FYEKFKDAVKEAFAKFWD

SEQ ID NO: 309 - protein, 18 aa, Artificial Sequence
  synthetic construct; Class A amphipathic helical peptides
FYEKFKDAVKDAFAKFWE

SEQ ID NO: 310 - protein, 18 aa, Artificial Sequence
  synthetic construct; Class A amphipathic helical peptides
FVEKFKEAAKDYFAKFWD

SEQ ID NO: 311 - protein, 18 aa, Artificial Sequence
  synthetic construct; Class A amphipathic helical peptides
FVDKFKDAAKEYFAKFWE

SEQ ID NO: 312 - protein, 18 aa, Artificial Sequence
  synthetic construct; Class A amphipathic helical peptides
FVDKFKEAAKDYFAKFWE

SEQ ID NO: 313 - protein, 18 aa, Artificial Sequence
  synthetic construct; Class A amphipathic helical peptides
FVEKFKDAAKEYFAKFWD

SEQ ID NO: 314 - protein, 18 aa, Artificial Sequence
  synthetic construct; Class A amphipathic helical peptides
FVEKFKDAAKDYFAKFWE

SEQ ID NO: 315 - protein, 18 aa, Artificial Sequence
  synthetic construct; Class A amphipathic helical peptides
FAEKYKEAVKDFFAKFWD

SEQ ID NO: 316 - protein, 18 aa, Artificial Sequence
  synthetic construct; Class A amphipathic helical peptides
FADKYKDAVKEFFAKFWE

SEQ ID NO: 317 - protein, 18 aa, Artificial Sequence
  synthetic construct; Class A amphipathic helical peptides
FADKYKEAVKDFFAKFWE

SEQ ID NO: 318 - protein, 18 aa, Artificial Sequence
  synthetic construct; Class A amphipathic helical peptides
FAEKYKDAVKEFFAKFWD

SEQ ID NO: 319 - protein, 18 aa, Artificial Sequence
  synthetic construct; Class A amphipathic helical peptides
FAEKYKDAVKDFFAKFWE

SEQ ID NO: 320 - protein, 18 aa, Artificial Sequence
  synthetic construct; Class A amphipathic helical peptides
FAEKVKEAFKDYFAKFWD

SEQ ID NO: 321 - protein, 18 aa, Artificial Sequence
  synthetic construct; Class A amphipathic helical peptides
FADKVKDAFKEYFAKFWE

SEQ ID NO: 322 - protein, 18 aa, Artificial Sequence
  synthetic construct; Class A amphipathic helical peptides
FADKVKEAFKDYFAKFWE

SEQ ID NO: 323 - protein, 18 aa, Artificial Sequence
  synthetic construct; Class A amphipathic helical peptides
FAEKVKDAFKEYFAKFWD

SEQ ID NO: 324 - protein, 18 aa, Artificial Sequence
  synthetic construct; Class A amphipathic helical peptides
FAEKVKDAFKDYFAKFWE

SEQ ID NO: 325 - protein, 18 aa, Artificial Sequence
  synthetic construct; Class A amphipathic helical peptides
FAEKFKEYVKDAFAKFWD

SEQ ID NO: 326 - protein, 18 aa, Artificial Sequence
  synthetic construct; Class A amphipathic helical peptides
FADKFKDYVKEAFAKFWE

SEQ ID NO: 327 - protein, 18 aa, Artificial Sequence
  synthetic construct; Class A amphipathic helical peptides
FADKFKEYVKDAFAKFWE

SEQ ID NO: 328 - protein, 18 aa, Artificial Sequence
  synthetic construct; Class A amphipathic helical peptides
FAEKFKDYVKEAFAKFWD

SEQ ID NO: 329 - protein, 18 aa, Artificial Sequence
  synthetic construct; Class A amphipathic helical peptides
FAEKFKDYVKDAFAKFWE

SEQ ID NO: 330 - protein, 18 aa, Artificial Sequence
  synthetic construct; Class A amphipathic helical peptides
FAEKFKEAFKDYVAKFWD

SEQ ID NO: 331 - protein, 18 aa, Artificial Sequence
  synthetic construct; Class A amphipathic helical peptides
FADKFKDAFKEYVAKFWE

SEQ ID NO: 332 - protein, 18 aa, Artificial Sequence
  synthetic construct; Class A amphipathic helical peptides
FADKFKEAFKDYVAKFWE

SEQ ID NO: 333 - protein, 18 aa, Artificial Sequence
  synthetic construct; Class A amphipathic helical peptides
FAEKFKDAFKEYVAKFWD

SEQ ID NO: 334 - protein, 18 aa, Artificial Sequence
  synthetic construct; Class A amphipathic helical peptides
FAEKFKDAFKDYVAKFWE

SEQ ID NO: 335 - protein, 18 aa, Artificial Sequence
  synthetic construct; Class A amphipathic helical peptides
FAEKFKEAFKDYFAKVWD

SEQ ID NO: 336 - protein, 18 aa, Artificial Sequence
  synthetic construct; Class A amphipathic helical peptides
FADKFKDAFKEYFAKVWE

SEQ ID NO: 337 - protein, 18 aa, Artificial Sequence
  synthetic construct; Class A amphipathic helical peptides
FADKFKEAFKDYFAKVWE

SEQ ID NO: 338 - protein, 18 aa, Artificial Sequence
  synthetic construct; Class A amphipathic helical peptides
FAEKFKDAFKEYFAKVWD

SEQ ID NO: 339 - protein, 18 aa, Artificial Sequence
  synthetic construct; Class A amphipathic helical peptides
FAEKFKDAFKDYFAKVWE

SEQ ID NO: 340 - protein, 18 aa, Artificial Sequence
  synthetic construct; Class A amphipathic helical peptides
FAEKFKEAVKDFFAKYWD

SEQ ID NO: 341 - protein, 18 aa, Artificial Sequence
  synthetic construct; Class A amphipathic helical peptides
FADKFKDAVKEFFAKYWE

SEQ ID NO: 342 - protein, 18 aa, Artificial Sequence
  synthetic construct; Class A amphipathic helical peptides
FADKFKEAVKDFFAKYWE

SEQ ID NO: 343 - protein, 18 aa, Artificial Sequence
  synthetic construct; Class A amphipathic helical peptides
FAEKFKDAVKEFFAKYWD

SEQ ID NO: 344 - protein, 18 aa, Artificial Sequence
  synthetic construct; Class A amphipathic helical peptides
FAEKFKDAVKDFFAKYWE

SEQ ID NO: 345 - protein, 18 aa, Artificial Sequence
  synthetic construct; Class A amphipathic helical peptides
WAEKFFEAVKDYFAKFKD

SEQ ID NO: 346 - protein, 18 aa, Artificial Sequence
  synthetic construct; Class A amphipathic helical peptides
WADKFFDAVKEYFAKFKE

SEQ ID NO: 347 - protein, 18 aa, Artificial Sequence
  synthetic construct; Class A amphipathic helical peptides
WADKFFEAVKDYFAKFKE

SEQ ID NO: 348 - protein, 18 aa, Artificial Sequence
  synthetic construct; Class A amphipathic helical peptides
WAEKFFDAVKEYFAKFKD

SEQ ID NO: 349 - protein, 18 aa, Artificial Sequence
  synthetic construct; Class A amphipathic helical peptides
WAEKFFDAVKDYFAKFKE

SEQ ID NO: 350 - protein, 18 aa, Artificial Sequence
  synthetic construct; Class A amphipathic helical peptides
FAEKWFEAVKDYFAKFKD

SEQ ID NO: 351 - protein, 18 aa, Artificial Sequence
  synthetic construct; Class A amphipathic helical peptides
FADKWFDAVKEYFAKFKE

SEQ ID NO: 352 - protein, 18 aa, Artificial Sequence
  synthetic construct; Class A amphipathic helical peptides
FADKWFEAVKDYFAKFKE

SEQ ID NO: 353 - protein, 18 aa, Artificial Sequence
  synthetic construct; Class A amphipathic helical peptides
FAEKWFDAVKEYFAKFKD

SEQ ID NO: 354 - protein, 18 aa, Artificial Sequence
  synthetic construct; Class A amphipathic helical peptides
FAEKWFDAVKDYFAKFKE

SEQ ID NO: 355 - protein, 18 aa, Artificial Sequence
  synthetic construct; Class A amphipathic helical peptides
FAEKFVEAWKDYFAKFKD

SEQ ID NO: 356 - protein, 18 aa, Artificial Sequence
  synthetic construct; Class A amphipathic helical peptides
FADKFVDAWKEYFAKFKE

SEQ ID NO: 357 - protein, 18 aa, Artificial Sequence
  synthetic construct; Class A amphipathic helical peptides
FADKFVEAWKDYFAKFKE

SEQ ID NO: 358 - protein, 18 aa, Artificial Sequence
  synthetic construct; Class A amphipathic helical peptides
FAEKFVDAWKEYFAKFKD

SEQ ID NO: 359 - protein, 18 aa, Artificial Sequence
  synthetic construct; Class A amphipathic helical peptides
FAEKFVDAWKDYFAKFKE

SEQ ID NO: 360 - protein, 18 aa, Artificial Sequence
  synthetic construct; Class A amphipathic helical peptides
FYEKFAEAVKDWFAKFKD

SEQ ID NO: 361 - protein, 18 aa, Artificial Sequence
  synthetic construct; Class A amphipathic helical peptides
FYDKFADAVKEWFAKFKE

SEQ ID NO: 362 - protein, 18 aa, Artificial Sequence
  synthetic construct; Class A amphipathic helical peptides
FYDKFAEAVKDWFAKFKE

SEQ ID NO: 363 - protein, 18 aa, Artificial Sequence
  synthetic construct; Class A amphipathic helical peptides
FYEKFADAVKEWFAKFKD

SEQ ID NO: 364 - protein, 18 aa, Artificial Sequence
  synthetic construct; Class A amphipathic helical peptides
FYEKFADAVKDWFAKFKE

SEQ ID NO: 365 - protein, 18 aa, Artificial Sequence
  synthetic construct; class A or class Y amphipathic helix peptide
DXXKYXXDKXYDKXKDYX

SEQ ID NO: 366 - protein, 18 aa, Artificial Sequence
  synthetic construct; peptide mimetic
DWFKHFYDKVAEKFKEAF

SEQ ID NO: 367 - protein, 18 aa, Artificial Sequence
  synthetic construct; peptide mimetic
EWFKHFYEKVADKFKDAF

SEQ ID NO: 368 - protein, 18 aa, Artificial Sequence
  synthetic construct; peptide mimetic
EWFKHFYDKVAEKFKEAF

SEQ ID NO: 369 - protein, 18 aa, Artificial Sequence
  synthetic construct; peptide mimetic
DWFKHFYEKVAEKFKEAF

SEQ ID NO: 370 - protein, 18 aa, Artificial Sequence
  synthetic construct; peptide mimetic
DWFKHFYDKVADKFKEAF

SEQ ID NO: 371 - protein, 18 aa, Artificial Sequence
  synthetic construct; peptide mimetic
DWFKHFYDKVAEKFKDAF

SEQ ID NO: 372 - protein, 18 aa, Artificial Sequence
  synthetic construct; peptide mimetic
DWHKFFYDKVAEKFKEAF

SEQ ID NO: 373 - protein, 18 aa, Artificial Sequence
  synthetic construct; peptide mimetic
EWHKFFYEKVADKFKDAF

SEQ ID NO: 374 - protein, 18 aa, Artificial Sequence
  synthetic construct; peptide mimetic
EWHKFFYDKVAEKFKEAF

SEQ ID NO: 375 - protein, 18 aa, Artificial Sequence
  synthetic construct; peptide mimetic
DWHKFFYEKVAEKFKEAF

SEQ ID NO: 376 - protein, 18 aa, Artificial Sequence
  synthetic construct; peptide mimetic
DWHKFFYDKVADKFKEAF

SEQ ID NO: 377 - protein, 18 aa, Artificial Sequence
  synthetic construct; peptide mimetic
DWHKFFYDKVAEKFKDAF

SEQ ID NO: 378 - protein, 18 aa, Artificial Sequence
  synthetic construct; peptide mimetic
DWFKFHYDKVAEKFKEAF

SEQ ID NO: 379 - protein, 18 aa, Artificial Sequence
  synthetic construct; peptide mimetic
EWFKFHYEKVADKFKDAF

SEQ ID NO: 380 - protein, 18 aa, Artificial Sequence
  synthetic construct; peptide mimetic
EWFKFHYDKVAEKFKEAF

SEQ ID NO: 381 - protein, 18 aa, Artificial Sequence
  synthetic construct; peptide mimetic
DWFKFHYEKVAEKFKEAF

SEQ ID NO: 382 - protein, 18 aa, Artificial Sequence
  synthetic construct; peptide mimetic
DWFKFHYDKVADKFKEAF

SEQ ID NO: 383 - protein, 18 aa, Artificial Sequence
  synthetic construct; peptide mimetic
DWFKFHYDKVAEKFKDAF

SEQ ID NO: 384 - protein, 18 aa, Artificial Sequence
  synthetic construct; peptide mimetic
DWFKVFYDKHAEKFKEAF

SEQ ID NO: 385 - protein, 18 aa, Artificial Sequence
  synthetic construct; peptide mimetic
EWFKVFYEKHADKFKDAF

SEQ ID NO: 386 - protein, 18 aa, Artificial Sequence
  synthetic construct; peptide mimetic
EWFKVFYDKHAEKFKEAF

SEQ ID NO: 387 - protein, 18 aa, Artificial Sequence
  synthetic construct; peptide mimetic
DWFKVFYEKHAEKFKEAF

SEQ ID NO: 388 - protein, 18 aa, Artificial Sequence
  synthetic construct; peptide mimetic
DWFKVFYDKHADKFKEAF

SEQ ID NO: 389 - protein, 18 aa, Artificial Sequence
  synthetic construct; peptide mimetic
DWFKVFYDKHAEKFKDAF

SEQ ID NO: 390 - protein, 18 aa, Artificial Sequence
  synthetic construct; peptide mimetic
DWFKAFYDKVAEKFKEHF

SEQ ID NO: 391 - protein, 18 aa, Artificial Sequence
  synthetic construct; peptide mimetic
EWFKAFYEKVADKFKDHF

SEQ ID NO: 392 - protein, 18 aa, Artificial Sequence
  synthetic construct; peptide mimetic
EWFKAFYDKVAEKFKEHF

SEQ ID NO: 393 - protein, 18 aa, Artificial Sequence
  synthetic construct; peptide mimetic
DWFKAFYEKVAEKFKEHF

SEQ ID NO: 394 - protein, 18 aa, Artificial Sequence
  synthetic construct; peptide mimetic
DWFKAFYDKVADKFKEHF

SEQ ID NO: 395 - protein, 18 aa, Artificial Sequence
  synthetic construct; peptide mimetic
DWFKAFYDKVAEKFKDHF

SEQ ID NO: 396 - protein, 18 aa, Artificial Sequence
  synthetic construct; peptide mimetic
DWFKAFYDKVAEKFKEFH

SEQ ID NO: 397 - protein, 18 aa, Artificial Sequence
  synthetic construct; peptide mimetic
EWFKAFYEKVADKFKDFH

SEQ ID NO: 398 - protein, 18 aa, Artificial Sequence
  synthetic construct; peptide mimetic
EWFKAFYDKVAEKFKEFH

SEQ ID NO: 399 - protein, 18 aa, Artificial Sequence
  synthetic construct; peptide mimetic
DWFKAFYDKVAEKFKEFH

SEQ ID NO: 400 - protein, 18 aa, Artificial Sequence
  synthetic construct; peptide mimetic
DWFKAFYEKVAEKFKEFH

SEQ ID NO: 401 - protein, 18 aa, Artificial Sequence
  synthetic construct; peptide mimetic
DWFKAFYDKVAEKFKEFH

SEQ ID NO: 402 - protein, 18 aa, Artificial Sequence
  synthetic construct; peptide mimetic
DWFKAFYDKVAEKFKDFH

SEQ ID NO: 403 - protein, 18 aa, Artificial Sequence
  synthetic construct; peptide mimetic
FAEKFKEAVKDYFAKFWD

SEQ ID NO: 404 - protein, 18 aa, Artificial Sequence
  synthetic construct; peptide mimetic
FHEKFKEAVKDYFAKFWD

SEQ ID NO: 405 - protein, 18 aa, Artificial Sequence
  synthetic construct; peptide mimetic
FHEKFKEAVKEYFAKFWE

SEQ ID NO: 406 - protein, 18 aa, Artificial Sequence
  synthetic construct; peptide mimetic
FHDKFKDAVKDYFAKFWD

SEQ ID NO: 407 - protein, 18 aa, Artificial Sequence
  synthetic construct; peptide mimetic
FHDKFKDAVKEYFAKFWE

SEQ ID NO: 408 - protein, 18 aa, Artificial Sequence
  synthetic construct; peptide mimetic
FHDKFKEAVKDYFAKFWD

SEQ ID NO: 409 - protein, 18 aa, Artificial Sequence
  synthetic construct; peptide mimetic
FHEKFKDAVKDYFAKFWD

SEQ ID NO: 410 - protein, 18 aa, Artificial Sequence
  synthetic construct; peptide mimetic
FHEKFKEAVKEYFAKFWD

SEQ ID NO: 411 - protein, 18 aa, Artificial Sequence
  synthetic construct; peptide mimetic
FHEKFKEAVKDYFAKFWE

SEQ ID NO: 412 - protein, 18 aa, Artificial Sequence
  synthetic construct; peptide mimetic
HFEKFKEAVKDYFAKFWD

SEQ ID NO: 413 - protein, 18 aa, Artificial Sequence
  synthetic construct; peptide mimetic
HFDKFKDAVKEYFAKFWE

SEQ ID NO: 414 - protein, 18 aa, Artificial Sequence
  synthetic construct; peptide mimetic
HFEKFKEAVKEYFAKFWE

SEQ ID NO: 415 - protein, 18 aa, Artificial Sequence
  synthetic construct; peptide mimetic
HFDKFKEAVKDYFAKFWD

SEQ ID NO: 416 - protein, 18 aa, Artificial Sequence
  synthetic construct; peptide mimetic
HFEKFKDAVKDYFAKFWD

SEQ ID NO: 417 - protein, 18 aa, Artificial Sequence
  synthetic construct; peptide mimetic
HFEKFKEAVKEYFAKFWD

SEQ ID NO: 418 - protein, 18 aa, Artificial Sequence
  synthetic construct; peptide mimetic
HFEKFKEAVKDYFAKFWE

SEQ ID NO: 419 - protein, 18 aa, Artificial Sequence
  synthetic construct; peptide mimetic
FFEKHKEAVKDYFAKFWD

SEQ ID NO: 420 - protein, 18 aa, Artificial Sequence
  synthetic construct; peptide mimetic
FFDKHKDAVKEYFAKFWE

SEQ ID NO: 421 - protein, 18 aa, Artificial Sequence
  synthetic construct; peptide mimetic
FFEKHKEAVKEYFAKFWE

SEQ ID NO: 422 - protein, 18 aa, Artificial Sequence
  synthetic construct; peptide mimetic
FFDKHKDAVKDYFAKFWD

SEQ ID NO: 423 - protein, 18 aa, Artificial Sequence
  synthetic construct; peptide mimetic
FFDKHKEAVKDYFAKFWD

SEQ ID NO: 424 - protein, 18 aa, Artificial Sequence
  synthetic construct; peptide mimetic
FFEKHKEAVKEYFAKFWD

SEQ ID NO: 425 - protein, 18 aa, Artificial Sequence
  synthetic construct; peptide mimetic
FFEKHKEAVKDYFAKFWE

SEQ ID NO: 426 - protein, 18 aa, Artificial Sequence
  synthetic construct; peptide mimetic
FVEKFKEAHKDYFAKFWD

SEQ ID NO: 427 - protein, 18 aa, Artificial Sequence
  synthetic construct; peptide mimetic
FVDKFKDAHKEYFAKFWE

SEQ ID NO: 428 - protein, 18 aa, Artificial Sequence
  synthetic construct; peptide mimetic
FVEKFKEAHKEYFAKFWE

SEQ ID NO: 429 - protein, 18 aa, Artificial Sequence
  synthetic construct; peptide mimetic
FVDKFKDAHKDYFAKFWD

SEQ ID NO: 430 - protein, 18 aa, Artificial Sequence
  synthetic construct; peptide mimetic
FVDKFKEAHKDYFAKFWD

SEQ ID NO: 431 - protein, 18 aa, Artificial Sequence
  synthetic construct; peptide mimetic
FVEKFKDAHKDYFAKFWD

SEQ ID NO: 432 - protein, 18 aa, Artificial Sequence
  synthetic construct; peptide mimetic
FVEKFKEAHKEYFAKFWD

SEQ ID NO: 433 - protein, 18 aa, Artificial Sequence
  synthetic construct; peptide mimetic
FVEKFKEAHKDYFAKFWE

SEQ ID NO: 434 - protein, 18 aa, Artificial Sequence
  synthetic construct; peptide mimetic
FAEKFKEHVKDYFAKFWD

SEQ ID NO: 435 - protein, 18 aa, Artificial Sequence
  synthetic construct; peptide mimetic
FADKFKDHVKEYFAKFWE

SEQ ID NO: 436 - protein, 18 aa, Artificial Sequence
  synthetic construct; peptide mimetic
FAEKFKEHVKEYFAKFWE

SEQ ID NO: 437 - protein, 18 aa, Artificial Sequence
  synthetic construct; peptide mimetic
FADKFKDHVKDYFAKFWD

SEQ ID NO: 438 - protein, 18 aa, Artificial Sequence
  synthetic construct; peptide mimetic
FADKFKEHVKDYFAKFWD

SEQ ID NO: 439 - protein, 18 aa, Artificial Sequence
  synthetic construct; peptide mimetic
FAEKFKDHVKDYFAKFWD

SEQ ID NO: 440 - protein, 18 aa, Artificial Sequence
  synthetic construct; peptide mimetic
FAEKFKEHVKEYFAKFWD

SEQ ID NO: 441 - protein, 18 aa, Artificial Sequence
  synthetic construct; peptide mimetic
FAEKFKEHVKDYFAKFWE

SEQ ID NO: 442 - protein, 18 aa, Artificial Sequence
  synthetic construct; peptide mimetic
FAEKFKEFVKDYHAKFWD

SEQ ID NO: 443 - protein, 18 aa, Artificial Sequence
  synthetic construct; peptide mimetic
FADKFKDFVKEYHAKFWE

SEQ ID NO: 444 - protein, 18 aa, Artificial Sequence
  synthetic construct; peptide mimetic
FADKFKEFVKDYHAKFWD

SEQ ID NO: 445 - protein, 18 aa, Artificial Sequence
  synthetic construct; peptide mimetic
FAEKFKDFVKDYHAKFWD

SEQ ID NO: 446 - protein, 18 aa, Artificial Sequence
  synthetic construct; peptide mimetic
FADKFKDFVKDYHAKFWD

SEQ ID NO: 447 - protein, 18 aa, Artificial Sequence
  synthetic construct; peptide mimetic
FAEKFKEFVKEYHAKFWE

SEQ ID NO: 448 - protein, 18 aa, Artificial Sequence
  synthetic construct; peptide mimetic
FAEKFKEFVKEYHAKFWD

SEQ ID NO: 449 - protein, 18 aa, Artificial Sequence
  synthetic construct; peptide mimetic
FAEKFKEFVKDYHAKFWE

SEQ ID NO: 450 - protein, 18 aa, Artificial Sequence
  synthetic construct; peptide mimetic
FAEKFKEFVKDYFAKHWD

SEQ ID NO: 451 - protein, 18 aa, Artificial Sequence
  synthetic construct; peptide mimetic
FADKFKDFVKEYFAKHWE

SEQ ID NO: 452 - protein, 18 aa, Artificial Sequence
  synthetic construct; peptide mimetic
FAEKFKEFVKEYFAKHWE

SEQ ID NO: 453 - protein, 18 aa, Artificial Sequence
  synthetic construct; peptide mimetic
FADKFKDFVKDYFAKHWD

SEQ ID NO: 454 - protein, 18 aa, Artificial Sequence
  synthetic construct; peptide mimetic
FADKFKEFVKDYFAKHWD

SEQ ID NO: 455 - protein, 18 aa, Artificial Sequence
  synthetic construct; peptide mimetic
FAEKFKDFVKDYFAKHWD

SEQ ID NO: 456 - protein, 18 aa, Artificial Sequence
  synthetic construct; peptide mimetic
FAEKFKEFVKEYFAKHWD

SEQ ID NO: 457 - protein, 18 aa, Artificial Sequence
  synthetic construct; peptide mimetic
FAEKFKEFVKDYFAKHWE

SEQ ID NO: 458 - protein, 18 aa, Artificial Sequence
  synthetic construct, Nph peptide mimetic
DWFKAFYDKVAEKFKEAF

SEQ ID NO: 459 - protein, 18 aa, Artificial Sequence
  synthetic construct, Nph peptide mimetic
EWFKAFYEKVADKFKDAF

SEQ ID NO: 460 - protein, 18 aa, Artificial Sequence
  synthetic construct, Nph peptide mimetic
EWFKAFYEKVAEKFKEAF

SEQ ID NO: 461 - protein, 18 aa, Artificial Sequence
  synthetic construct, Nph peptide mimetic
DWFKAFYDKVADKFKDAF

SEQ ID NO: 462 - protein, 18 aa, Artificial Sequence
  synthetic construct, Nph peptide mimetic
EWFKAFYDKVAEKFKEAF

SEQ ID NO: 463 - protein, 18 aa, Artificial Sequence
  synthetic construct, Nph peptide mimetic
DWFKAFYEKVAEKFKEAF

SEQ ID NO: 464 - protein, 18 aa, Artificial Sequence
  synthetic construct, Nph peptide mimetic
DWFKAFYDKVADKFKEAF

SEQ ID NO: 465 - protein, 18 aa, Artificial Sequence
  synthetic construct, Nph peptide mimetic
DWFKAFYDKVAEKFKDAF

SEQ ID NO: 466 - protein, 18 aa, Artificial Sequence
  synthetic construct, Nph peptide mimetic
DWFKAFYDKVAEKFKEAF

SEQ ID NO: 467 - protein, 18 aa, Artificial Sequence
  synthetic construct, Nph peptide mimetic
DWFKAFYDKVAEKFKEAF

SEQ ID NO: 468 - protein, 18 aa, Artificial Sequence
  synthetic construct, Nph peptide mimetic
DWFKAFYDKVAEKFKEAF

SEQ ID NO: 469 - protein, 18 aa, Artificial Sequence
  synthetic construct, Nph peptide mimetic
DWFKAFYDKVAEKFKEAF

SEQ ID NO: 470 - protein, 18 aa, Artificial Sequence
  synthetic construct, Nph peptide mimetic
DWFKAFYDKVAEKFKEAF

SEQ ID NO: 471 - protein, 18 aa, Artificial Sequence
  synthetic construct, Nph peptide mimetic
DWFKAFYDKVAEKFKEAF

SEQ ID NO: 472 - protein, 18 aa, Artificial Sequence
  synthetic construct, Nph peptide mimetic
FAEKFKEAVKDYFAKFWD

SEQ ID NO: 473 - protein, 18 aa, Artificial Sequence
  synthetic construct, Nph peptide mimetic
FAEKFKEAVKDYFAKFWD

SEQ ID NO: 474 - protein, 18 aa, Artificial Sequence
  synthetic construct, Nph peptide mimetic
FAEKFKEAVKDYFAKFWD

SEQ ID NO: 475 - protein, 18 aa, Artificial Sequence
  synthetic construct, Nph peptide mimetic
FAEKFKEAVKDYFAKFWD

SEQ ID NO: 476 - protein, 18 aa, Artificial Sequence
  synthetic construct, Nph peptide mimetic
FAEKFKEAVKDYFAKFWD

SEQ ID NO: 477 - protein, 18 aa, Artificial Sequence
  synthetic construct, Nph peptide mimetic
FAEKFKEAVKDYFAKFWD

SEQ ID NO: 478 - protein, 18 aa, Artificial Sequence
  synthetic construct, Nph peptide mimetic
FAEKEKEAVKDYFAKFWD

SEQ ID NO: 479 - protein, 18 aa, Artificial Sequence
  synthetic construct; peptide mimetic
FAEKFKEAVKEYFAKFWE

SEQ ID NO: 480 - protein, 18 aa, Artificial Sequence
  synthetic construct; peptide mimetic
FADKFKDAVKDYFAKFWD

SEQ ID NO: 481 - protein, 18 aa, Artificial Sequence
  synthetic construct; peptide mimetic
FAERFREAVKDYFAKFWD

SEQ ID NO: 482 - protein, 18 aa, Artificial Sequence
  synthetic construct; peptide mimetic
FAEKFREAVKDYFAKFWD

SEQ ID NO: 483 - protein, 18 aa, Artificial Sequence
  synthetic construct; peptide mimetic
FAEKFKEAVRDYFAKFWD

SEQ ID NO: 484 - protein, 18 aa, Artificial Sequence
  synthetic construct; peptide mimetic
FAEKFKEAVKDYFARFWD

SEQ ID NO: 485 - protein, 18 aa, Artificial Sequence
  synthetic construct; peptide mimetic
FAEKFREAVKDYEAKFWD

SEQ ID NO: 486 - protein, 18 aa, Artificial Sequence
  synthetic construct; peptide mimetic
LFEKFAEAFKDYVAKWKD

SEQ ID NO: 487 - protein, 18 aa, Artificial Sequence
  synthetic construct; peptide mimetic
LFERFAEAFKDYVAKWKD

SEQ ID NO: 488 - protein, 18 aa, Artificial Sequence
  synthetic construct; peptide mimetic
LFEKFAEAFRDYVAKWKD

SEQ ID NO: 489 - protein, 18 aa, Artificial Sequence
  synthetic construct; peptide mimetic
LFEKFAEAFKDYVARWKD

SEQ ID NO: 490 - protein, 18 aa, Artificial Sequence
  synthetic construct; peptide mimetic
LFEKFAEAFKDYVAKWRD

SEQ ID NO: 491 - protein, 18 aa, Artificial Sequence
  synthetic construct; peptide mimetic
LFEKFAEAFKEYVAKWKE

SEQ ID NO: 492 - protein, 18 aa, Artificial Sequence
  synthetic construct; peptide mimetic
LFDKFADAFKDYVAKWKD

SEQ ID NO: 493 - protein, 18 aa, Artificial Sequence
  synthetic construct; peptide mimetic
LFDKFAEAFKDYVAKWKD

SEQ ID NO: 494 - protein, 18 aa, Artificial Sequence
  synthetic construct; peptide mimetic
LFEKFADAFKDYVAKWKD

SEQ ID NO: 495 - protein, 18 aa, Artificial Sequence
  synthetic construct; peptide mimetic
LFEKFAEAFKEYVAKWKD

SEQ ID NO: 496 - protein, 18 aa, Artificial Sequence
  synthetic construct; peptide mimetic
LFEKFAEAFKDYVAKWKE

SEQ ID NO: 497 - protein, 18 aa, Artificial Sequence
  synthetic construct; peptide mimetic
FAEKAWEFVKDYFAKLKD

SEQ ID NO: 498 - protein, 18 aa, Artificial Sequence
  synthetic construct; peptide mimetic
FAERAWEFVKDYFAKLKD

SEQ ID NO: 499 - protein, 18 aa, Artificial Sequence
  synthetic construct; peptide mimetic
FAEKAWEFVKDYFAKLKD

SEQ ID NO: 500 - protein, 18 aa, Artificial Sequence
  synthetic construct; peptide mimetic
FAEKAWEFVKDYFAKLKD

SEQ ID NO: 501 - protein, 18 aa, Artificial Sequence
  synthetic construct; peptide mimetic
FAEKAWEFVKDYFAKLRD

SEQ ID NO: 502 - protein, 18 aa, Artificial Sequence
  synthetic construct; peptide mimetic
FAEKAWEFVKEYFAKLKE

SEQ ID NO: 503 - protein, 18 aa, Artificial Sequence
  synthetic construct; peptide mimetic
FADKAWDFVKDYFAKLKD

SEQ ID NO: 504 - protein, 18 aa, Artificial Sequence
  synthetic construct; peptide mimetic
FADKAWEFVKDYFAKLKD

SEQ ID NO: 505 - protein, 18 aa, Artificial Sequence
  synthetic construct; peptide mimetic
FAEKAWDFVKDYFAKLKD

SEQ ID NO: 506 - protein, 18 aa, Artificial Sequence
  synthetic construct; peptide mimetic
FAEKAWEFVKEYFAKLKD

SEQ ID NO: 507 - protein, 18 aa, Artificial Sequence
  synthetic construct; peptide mimetic
FAEKAWEFVKDYFAKLKE

SEQ ID NO: 508 - protein, 18 aa, Artificial Sequence
  synthetic construct; peptide mimetic
FFEKFKEFVKDYFAKLWD

SEQ ID NO: 509 - protein, 18 aa, Artificial Sequence
  synthetic construct; peptide mimetic
FFEKFKEFVKEYFAKLWE

SEQ ID NO: 510 - protein, 18 aa, Artificial Sequence
  synthetic construct; peptide mimetic
FFDKFKDFVKDYFAKLWD

SEQ ID NO: 511 - protein, 18 aa, Artificial Sequence
  synthetic construct; peptide mimetic
FFERFKEFVKDYFAKLWD

SEQ ID NO: 512 - protein, 18 aa, Artificial Sequence
  synthetic construct; peptide mimetic
FFEKFREFVKDYFAKLWD

SEQ ID NO: 513 - protein, 18 aa, Artificial Sequence
  synthetic construct; peptide mimetic
FFEKFKEFVRDYFAKLWD

SEQ ID NO: 514 - protein, 18 aa, Artificial Sequence
  synthetic construct; peptide mimetic
FFEKFKEFVKDYFARLWD

SEQ ID NO: 515 - protein, 18 aa, Artificial Sequence
  synthetic construct; peptide mimetic
FFDKFKEFVKDYFAKLWD

SEQ ID NO: 516 - protein, 18 aa, Artificial Sequence
  synthetic construct; peptide mimetic
FFEKFKDFVKDYFAKLWD

SEQ ID NO: 517 - protein, 18 aa, Artificial Sequence
  synthetic construct; peptide mimetic
FFEKFKEFVKEYFAKLWD

SEQ ID NO: 518 - protein, 18 aa, Artificial Sequence
  synthetic construct; peptide mimetic
FFEKFKEFVKDYFAKLWE

SEQ ID NO: 519 - protein, 18 aa, Artificial Sequence
  synthetic construct; peptide mimetic
FLEKFKEFVKDYFAKFWD

SEQ ID NO: 520 - protein, 18 aa, Artificial Sequence
  synthetic construct; peptide mimetic
FLEKFKEFVKEYFAKFWE

SEQ ID NO: 521 - protein, 18 aa, Artificial Sequence
  synthetic construct; peptide mimetic
FLDKFKEFVKDYFAKFWD

SEQ ID NO: 522 - protein, 18 aa, Artificial Sequence
  synthetic construct; peptide mimetic
FLDKFKEFVKDYFAKFWD

SEQ ID NO: 523 - protein, 18 aa, Artificial Sequence
  synthetic construct; peptide mimetic
FLEKFKDFVKDYFAKFWD

SEQ ID NO: 524 - protein, 18 aa, Artificial Sequence
  synthetic construct; peptide mimetic
FLEKFKEFVKEYFAKFWD

SEQ ID NO: 525 - protein, 18 aa, Artificial Sequence
  synthetic construct; peptide mimetic
FLEKFKEFVKDYFAKFWE

SEQ ID NO: 526 - protein, 18 aa, Artificial Sequence
  synthetic construct; peptide mimetic
FLERFKEFVKDYFAKFWD

SEQ ID NO: 527 - protein, 18 aa, Artificial Sequence
  synthetic construct; peptide mimetic
FLEKFREFVKDYFAKFWD

SEQ ID NO: 528 - protein, 18 aa, Artificial Sequence
  synthetic construct; peptide mimetic
FLEKFKEFVRDYFAKFWD

SEQ ID NO: 529 - protein, 18 aa, Artificial Sequence
  synthetic construct; peptide mimetic
FLEKFKEFVKDYFARFWD

SEQ ID NO: 530 - protein, 18 aa, Artificial Sequence
  synthetic construct; peptide mimetic
FFEKFKEFFKDYFAKLWD

SEQ ID NO: 531 - protein, 18 aa, Artificial Sequence
  synthetic construct; peptide mimetic
FFEKFKEFFKEYFAKLWE

SEQ ID NO: 532 - protein, 18 aa, Artificial Sequence
  synthetic construct; peptide mimetic
FFDKFKDFFKDYFAKLWD

SEQ ID NO: 533 - protein, 18 aa, Artificial Sequence
  synthetic construct; peptide mimetic
FFERFKEFFKDYFAKLWD

SEQ ID NO: 534 - protein, 18 aa, Artificial Sequence
  synthetic construct; peptide mimetic
FFEKFREFFKDYFAKLWD

SEQ ID NO: 535 - protein, 18 aa, Artificial Sequence
  synthetic construct; peptide mimetic
FFEKFKEFFRDYFAKLWD

SEQ ID NO: 536 - protein, 18 aa, Artificial Sequence
  synthetic construct; peptide mimetic
FFERFKEFFKDYFARLWD

SEQ ID NO: 537 - protein, 18 aa, Artificial Sequence
  synthetic construct; peptide mimetic
FFDKFKEFFKDYFAKLWD

SEQ ID NO: 538 - protein, 18 aa, Artificial Sequence
  synthetic construct; peptide mimetic
FFEKEKDFFKDYFAKLWD

SEQ ID NO: 539 - protein, 18 aa, Artificial Sequence
  synthetic construct; peptide mimetic
FFEKFKEFFKEYFAKLWD

SEQ ID NO: 540 - protein, 18 aa, Artificial Sequence
  synthetic construct; peptide mimetic
FFEKFKEFFKDYFAKLWE

SEQ ID NO: 541 - protein, 18 aa, Artificial Sequence
  synthetic construct; peptide mimetic
FAEKFKEAVKDYFAKFWD

SEQ ID NO: 542 - protein, 18 aa, Artificial Sequence
  synthetic construct; peptide mimetic
DKWKAVYDKFAEAFKEFF

SEQ ID NO: 543 - protein, 18 aa, Artificial Sequence
  synthetic construct; peptide mimetic
EKWKAVYEKFAEAFKEFF

SEQ ID NO: 544 - protein, 18 aa, Artificial Sequence
  synthetic construct; peptide mimetic
DKWKAVYDKFADAFKDFF

SEQ ID NO: 545 - protein, 18 aa, Artificial Sequence
  synthetic construct; peptide mimetic
DRWKAVYDKFAEAFKEFF

SEQ ID NO: 546 - protein, 18 aa, Artificial Sequence
  synthetic construct; peptide mimetic
DKWRAVYDKFAEAFKEFF

SEQ ID NO: 547 - protein, 18 aa, Artificial Sequence
  synthetic construct; peptide mimetic
DKWKAVYDRFAEAFKEFF

SEQ ID NO: 548 - protein, 18 aa, Artificial Sequence
  synthetic construct; peptide mimetic
DKWKAVYDKFAEAFREFF

SEQ ID NO: 549 - protein, 18 aa, Artificial Sequence
  synthetic construct; peptide mimetic
FFEKFAEAFKDYVAKWKD

SEQ ID NO: 550 - protein, 18 aa, Artificial Sequence
  synthetic construct; peptide mimetic
FFEKFAEAFKEYVAKWKE

SEQ ID NO: 551 - protein, 18 aa, Artificial Sequence
  synthetic construct; peptide mimetic
FFDKFADAFKDYVAKWKD

SEQ ID NO: 552 - protein, 18 aa, Artificial Sequence
  synthetic construct; peptide mimetic
FFERFAEAFKDYVAKWKD

SEQ ID NO: 553 - protein, 18 aa, Artificial Sequence
  synthetic construct; peptide mimetic
EFERFAEAFRDYVAKWKD

SEQ ID NO: 554 - protein, 18 aa, Artificial Sequence
  synthetic construct; peptide mimetic
FFEKFAEAFKDYVARWKD

SEQ ID NO: 555 - protein, 18 aa, Artificial Sequence
  synthetic construct; peptide mimetic
FFERFAEAFKDYVAKWRD

SEQ ID NO: 556 - protein, 18 aa, Artificial Sequence
  synthetic construct; peptide mimetic
FFDKFAEAFKDYVAKWKD

SEQ ID NO: 557 - protein, 18 aa, Artificial Sequence
  synthetic construct; peptide mimetic
FFEKFADAFKDYVAKWKD

SEQ ID NO: 558 - protein, 18 aa, Artificial Sequence
  synthetic construct; peptide mimetic
FFERFAEAFKEYVAKWKD

SEQ ID NO: 559 - protein, 18 aa, Artificial Sequence
  synthetic construct; peptide mimetic
FFERFAEAFKDYVAKWKE

SEQ ID NO: 560 - protein, 18 aa, Artificial Sequence
  synthetic construct; peptide mimetic
FFEKFKEFFKDYFAKFWD

SEQ ID NO: 561 - protein, 18 aa, Artificial Sequence
  synthetic construct; peptide mimetic
FFDKFKDFFKDYFAKFWD

SEQ ID NO: 562 - protein, 18 aa, Artificial Sequence
  synthetic construct; peptide mimetic
FFEKFKEFFKEYFAKFWE

SEQ ID NO: 563 - protein, 18 aa, Artificial Sequence
  synthetic construct; peptide mimetic
FFERFKEFFKDYFAKFWD

SEQ ID NO: 564 - protein, 18 aa, Artificial Sequence
  synthetic construct; peptide mimetic
FFEKFREFFKDYFAKFWD

SEQ ID NO: 565 - protein, 18 aa, Artificial Sequence
  synthetic construct; peptide mimetic
FFEKFKEFFRDYFAKFWD

SEQ ID NO: 566 - protein, 18 aa, Artificial Sequence
  synthetic construct; peptide mimetic
FFEKFKEFFKDYFARFWD

SEQ ID NO: 567 - protein, 18 aa, Artificial Sequence
  synthetic construct; peptide mimetic
FFDKFKEFFKDYFAKFWD

SEQ ID NO: 568 - protein, 18 aa, Artificial Sequence
  synthetic construct; peptide mimetic
FFEKFKDFFKDYFAKFWD

SEQ ID NO: 569 - protein, 18 aa, Artificial Sequence
  synthetic construct; peptide mimetic
FFEKFKEFFKEYFAKFWD

SEQ ID NO: 570 - protein, 18 aa, Artificial Sequence
  synthetic construct; peptide mimetic
FFEKFKEFFKDYFAKFWE

SEQ ID NO: 571 - protein, 18 aa, Artificial Sequence
  synthetic construct; epsilon-methylLys analog
DWFKAFYDKVAEKFKEAF

SEQ ID NO: 572 - protein, 18 aa, Artificial Sequence
  synthetic construct; epsilon-methylLys analog
DWFKAFYDKVAEKFKEAF

SEQ ID NO: 573 - protein, 18 aa, Artificial Sequence
  synthetic construct; epsilon-N-dimethylLys analog
DWFKAFYDKVAEKFKEAF

SEQ ID NO: 574 - protein, 18 aa, Artificial Sequence
  synthetic construct; epsilon-N-dimethylLys analog
DWFKAFYDKVAEKFKEAF

SEQ ID NO: 575 - protein, 18 aa, Artificial Sequence
  synthetic construct; epsilon-N-dimethylLys analog
DWFKAFYDKVAEKFKEAF

SEQ ID NO: 576 - protein, 18 aa, Artificial Sequence
  synthetic construct; epsilon-N-diethylLys analog
DWFKAFYDKVAEKFKEAF

SEQ ID NO: 577 - protein, 18 aa, Artificial Sequence
  synthetic construct; epsilon-N-diethylLys analog
DWFKAFYDKVAEKFKEAF

SEQ ID NO: 578 - protein, 18 aa, Artificial Sequence
  synthetic construct; epsilon-N-diethylLys analog
DWFKAFYDKVAEKFKEAF

SEQ ID NO: 579 - protein, 18 aa, Artificial Sequence
  synthetic construct; epsilon-N-monomethylLys analog
DWFKAFYDKVAEKFKEAF

SEQ ID NO: 580 - protein, 18 aa, Artificial Sequence
  synthetic construct; epsilon-N-monomethylLys analog
DWFKAFYDKVAEKFKEAF

SEQ ID NO: 581 - protein, 18 aa, Artificial Sequence
  synthetic construct; epsilon-N-monomethylLys analog
DWFKAFYDKVAEKFKEAF

SEQ ID NO: 582 - protein, 18 aa, Artificial Sequence
  synthetic construct; epsilon-N-ethylLys analog
DWFKAFYDKVAEKFKEAF

SEQ ID NO: 583 - protein, 18 aa, Artificial Sequence
  synthetic construct; epsilon-N-ethylLys analog
DWFKAFYDKVAEKFKEAF

SEQ ID NO: 584 - protein, 18 aa, Artificial Sequence
  synthetic construct; epsilon-N-ethylLys analog
DWFKAFYDKVAEKFKEAF

SEQ ID NO: 585 - protein, 18 aa, Artificial Sequence
  synthetic construct; homoLys analog
DWFKAFYDKVAEKFKEAF

SEQ ID NO: 586 - protein, 18 aa, Artificial Sequence
  synthetic construct; homoLys analog
DWFKAFYDKVAEKFKEAF

SEQ ID NO: 587 - protein, 18 aa, Artificial Sequence
  synthetic construct; homoLys analog
DWFKAFYDKVAEKFKEAF

SEQ ID NO: 588 - protein, 18 aa, Artificial Sequence
  synthetic construct; homoLys analog
DWFKAFYDKVAEKFKEAF

SEQ ID NO: 589 - protein, 18 aa, Artificial Sequence
  synthetic construct; homoLys analog
DWFKAFYDKVAEKFKEAF

SEQ ID NO: 590 - protein, 18 aa, Artificial Sequence
  synthetic construct; homoLys analog
DWFKAFYDKVAEKFKEAF

SEQ ID NO: 591 - protein, 18 aa, Artificial Sequence
  synthetic construct; homoLys analog
DWFKAFYDKVAEKFKEAF

SEQ ID NO: 592 - protein, 18 aa, Artificial Sequence
  synthetic construct; homoLys analog
DWFKAFYDKVAEKFKEAF

SEQ ID NO: 593 - protein, 18 aa, Artificial Sequence
  synthetic construct; homoLys analog
DWFKAFYDKVAEKFKEAF

SEQ ID NO: 594 - protein, 18 aa, Artificial Sequence
  synthetic construct; homoLys analog
DWFKAFYDKVAEKFKEAF

SEQ ID NO: 595 - protein, 18 aa, Artificial Sequence
  synthetic construct; homoLys analog
DWFKAFYDKVAEKFKEAF

SEQ ID NO: 596 - protein, 18 aa, Artificial Sequence
  synthetic construct; ornithine analogs
DWFXAFYDXVAEXFXEAF

SEQ ID NO: 597 - protein, 18 aa, Artificial Sequence
  synthetic construct; ornithine analogs
DWFXAFYDXVAEXFXEAF

SEQ ID NO: 598 - protein, 18 aa, Artificial Sequence
  synthetic construct; ornithine analogs
DWFXAFYDXVAEXFXEAF

SEQ ID NO: 599 - protein, 18 aa, Artificial Sequence
  synthetic construct; ornithine analogs
DWFXAFYDXVAEXFXEAF

SEQ ID NO: 600 - protein, 18 aa, Artificial Sequence
  synthetic construct; ornithine analogs
DWFXAFYDXVAEXFXEAF

SEQ ID NO: 601 - protein, 18 aa, Artificial Sequence
  synthetic construct; ornithine analogs
DWFXAFYDXVAEXFXEAF

SEQ ID NO: 602 - protein, 18 aa, Artificial Sequence
  synthetic construct; ornithine analogs
DWFXAFYDXVAEXFXEAF

SEQ ID NO: 603 - protein, 18 aa, Artificial Sequence
  synthetic construct; ornithine analogs
DWFXAFYDXVAEXFXEAF

SEQ ID NO: 604 - protein, 18 aa, Artificial Sequence
  synthetic construct; ornithine analogs
DWFXAFYDXVAEXFXEAF

SEQ ID NO: 605 - protein, 18 aa, Artificial Sequence
  synthetic construct; ornithine analogs
DWFXAFYDXVAEXFXEAF

SEQ ID NO: 606 - protein, 18 aa, Artificial Sequence
  synthetic construct; ornithine analogs
DWFXAFYDXVAEXFXEAF

SEQ ID NO: 607 - protein, 10 aa, Artificial Sequence
  synthetic construct; receptor binding domains of ApoE
RLLRKRLKRL

SEQ ID NO: 608 - protein, 10 aa, Artificial Sequence
  synthetic construct; receptor binding domain of ApoE
RMLRKRMKRL

SEQ ID NO: 609 - protein, 10 aa, Artificial Sequence
  synthetic construct; receptor binding domain of ApoE
RLLRKPLKRL

SEQ ID NO: 610 - protein, 10 aa, Artificial Sequence
  synthetic construct; receptor binding domain of ApoE
RVLRKRVNRL

SEQ ID NO: 611 - protein, 10 aa, Artificial Sequence
  synthetic construct; receptor binding domain of ApoE
RLVRKRLKRM

SEQ ID NO: 612 - protein, 10 aa, Artificial Sequence
  synthetic construct; receptor binding domain of ApoE
RLLRRRLRRL

SEQ ID NO: 613 - protein, 10 aa, Artificial Sequence
  synthetic construct; receptor binding domain of ApoE
RFFRKRLKRL

SEQ ID NO: 614 - protein, 18 aa, Artificial Sequence
  synthetic construct; Lipid-Associating Peptide
FAEKLKEAVKDYFAKLWD

SEQ ID NO: 615 - protein, 18 aa, Artificial Sequence
  synthetic construct; Lipid-Associating Peptide
FAEKFKEAVKDYFAKFWD

SEQ ID NO: 616 - protein, 18 aa, Artificial Sequence
  synthetic construct; Lipid-Associating Peptide
KAFEEVLAKKFYDKALWD

SEQ ID NO: 617 - protein, 10 aa, Artificial Sequence
  synthetic construct; receptor binding domain of ApoE
LRLLRKLKRR

SEQ ID NO: 618 - protein, 18 aa, Artificial Sequence
  synthetic construct; peptide mimetic
DWLKAFYDKVFEKFKEAF

SEQ ID NO: 619 - protein, 18 aa, Artificial Sequence
  synthetic construct; peptide mimetic
DWLKAFYDKVAEKFKEFF

(19) United States
(12) Patent Application Publication
(10) Pub. No.: US 2016/0002315 A1
(43) Pub. Date: Jan. 7, 2016

(54) APOLIPOPROTEIN MIMETICS AND USES THEREOF

(71) Applicant: UAB RESEARCH FOUNDATION, Birmingham, AL (US)

(72) Inventors: G.M. Anantharamaiah, Birmingham, AL (US); Dennis Goldberg, Sudbury, MA (US)

(73) Assignee: UAB RESEARCH FOUNDATION, Birmingham, AL (US)

(21) Appl. No.: 14/770,270

(22) PCT Filed: Mar. 14, 2014
(86) PCT No.: PCT/US14/27719

§ 371 Date: Aug. 25, 2015

Related U.S. Application Data
(60) Provisional application No. 61/782,956, filed on Mar. 14, 2013.
(60) Provisional application No. 61/834,992, filed on Jun. 14, 2013.

(51) Int. Cl. C07K 14/775

(52) U.S. Cl. CPC C07K 14/775

(57) ABSTRACT

The disclosure provides dosing regimens and methods for treating atherosclerosis with an effective amount of Apo E mimetic to provide sustained therapeutic effects even after withdrawal of the treatment. The dosing regimens and methods involve a treatment cycle followed by a rest phase wherein a subject is administered an effective amount of an Apo E mimetic during the treatment cycle and no Apo E mimetic during the rest phase. The treatment cycle and the rest phase can vary.

### CROSS-REFERENCE TO RELATED APPLICATIONS

This application claims benefit of U.S. Provisional Application No. 61/782,956, filed Mar. 14, 2013 and U.S. Provisional Application No. 61/834,992 filed Jun. 14, 2013, both of which are hereby incorporated herein by reference in their entirety.

### STATEMENT REGARDING FEDERALLY SPONSORED RESEARCH

This invention was made with government support under R01 HL090803 awarded by The National Institutes of Health. The government has certain rights in the invention.

### REFERENCE TO SEQUENCE LISTING

The Sequence Listing submitted Mar. 14, 2014 as a text file named “21085_{—}0184P1_Sequence_Listing.txt,” created on Mar. 14, 2014, and having a size of 206,473 bytes is hereby incorporated by reference pursuant to 37 C.F.R. §1.52(e)(5).

### FIELD OF THE INVENTION

The disclosed invention relates generally to the treatment of atherosclerosis. In particular, the treatment pertains to a specific dosing regimen that includes a treatment cycle followed by a rest phase that provides prolonged therapeutic effects even after the treatment is withdrawn.

### BACKGROUND

In the United States, heart disease is the leading cause of death in both men and women. Several causative factors are implicated in the development of cardiovascular disease including hereditary predisposition to the disease, gender, lifestyle factors such as smoking and diet, age, hypertension, and hyperlipidemia, including hypercholesterolemia. Several of these factors, particularly hyperlipidemia and hypercholesteremia (high blood cholesterol concentrations) provide a significant risk factor associated with atherosclerosis.

Atherosclerosis is associated with an inflammatory response caused by the accumulation of low-density lipoprotein (LDL) molecules in blood vessels. It can be asymptomatic for years. Atherosclerosis causes hardening and narrowing of blood vessels. There are several treatments for atherosclerosis, such as lifestyle change, medication and medical procedures. Statins are a well-known treatment for atherosclerosis. Statins have proven to reduce cardiac risk however the withdrawal of statin therapy abrogrates the protective effect (Heeschen et al. Circulation. 105:1446-1452, 2002).

The current approach to treating atherosclerosis is to provide earlier intervention and life-long treatment. This approach is problematic as it requires identifying asymptomatic patients early in their life cycle and, since risk increases with age, maintaining therapy for the duration of their life. Further, the most efficacious currently available therapies are unable to prevent major cardiac events in all patients whether as primary or secondary interventions. Therefore, there is a need for therapies that can provide rapid benefit in reducing atherosclerosis and have long-term effects that do not require constant administration. The compositions and methods disclosed herein provide an atherosclerosis therapy with sustained therapeutic effects even after the treatment is withdrawn.

### BRIEF SUMMARY

Dosing regimens containing at least one treatment cycle followed by a rest phase are provided. Also provided are methods of treating atherosclerosis involving administering to a subject an effective amount of an Apo E mimetic for at least one treatment cycle followed by a rest phase. The treatment cycles and rest phases of the dosing regimens and the methods of treating can be the same.

In both the dosing regimens and the methods of treating, the treatment cycle contains the administration of an effective amount of an Apo E mimetic to cause an immediate benefit illustrated by a decrease in atherosclerosis, a decrease in artery wall stiffness, a decrease in isolated systolic hypertension, a decrease in arterial inflammation, an increase in anti-oxidant capability of the HDL fraction and/or an improvement in myocardial function, and to allow for a sustained therapeutic effect after withdrawal of the Apo E mimetic. The treatment cycle can include the administration of an effective amount of the Apo E mimetic once a week for four weeks or once a week for three months. In some instances, the treatment cycle includes the administration of an effective amount of the Apo E mimetic once every two weeks for up to 12 weeks.

The rest phase is a period of time wherein the Apo E mimetic is not administered. The rest phase can be at least four weeks. An atherosclerosis therapeutic other than an Apo E mimetic can be administered during the rest phase. The atherosclerosis therapeutic other than an Apo E mimetic can be a conventional lipid lowering therapy, such as a statin or bile acid sequestrant, and/or a therapeutic such as a PCSK9 inhibitor, a VLDL synthesis inhibitor and/or a CETP inhibitor.

Both the dosing regimen and the methods of treating can further include a second treatment cycle after the rest phase. The second treatment cycle can be administered after a four week rest phase or one or more years from the end of the initial treatment cycle.

The Apo E mimetic can be hE18A (LRKLRKRLLRDWLKAFYDKVAEKLKEAF (SEQ ID NO:1)).

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A, 1B, and 1C show the design of mR18L. A) 18L; B) m18L; and C) mR18L.

FIG. 2 is a schematic diagram of the experimental protocol used in Apo E null mice.

FIGS. 3A and 3B show the amount of plasma cholesterol (mg/dl). A) Bar graph showing the plasma cholesterol after six weeks of western diet compared to after two weeks of chow after the six weeks of western diet. B) Line graph showing the plasma cholesterol over a 30 day period of treatment with mR18L, hE18A or control.

FIG. 4 is a bar graph of plasma triglycerides (mg/dl) versus different treatments (saline, mR18L or hE18A).

FIGS. 5A and 5B are bar graphs of reactive oxygen species (ROS) and paraoxanase (PON) versus the different treatments, respectively.

FIGS. 6A and 6B are a combination of a line graph showing the absorbance in the different elutions and polyacrylamide gels were stained to show PON-1 activity in the HDL fractions (29-32). A) Control treated; B) Ac-he18A-NH₂ treated. The plasma cholesterol lipoprotein fractions were analyzed by the CLiP method. The solid lines show the cholesterol content of the various lipoprotein fractions, displayed as dashed lines.

FIG. 7 is a line graph showing the plasma cholesterol levels (mg/dl) over a 60 day time period. Peptide was administered during the first 30 days The final time point is four weeks after the end of treatment with saline, mR18L or hE18A. Cholesterol levels in all treatment groups were the same at the end of the study.

FIGS. 8A and 8B are bar graphs of PON activity versus different treatments and ROS versus different treatments, respectively. The samples were from four weeks after treatment.

FIGS. 9A and 9B show that aortic lesions were reduced in hE18A treated mice even after treatment had been withdrawn for four weeks. A) Bar graph showing percent lesion area versus different treatments. B) Stained tissue samples showing the en face lesion area.

FIG. 10 shows that lesions in the aortic sinus were reduced in hE18A treated mice even after treatment had been withdrawn for four weeks. mR18L had no effect on aortic sinus lesions at study termination.

FIG. 11 shows a trend towards decreased macrophage coverage in the aortic sinus of mice treated with hE18A, relative to the other treatment groups, at study termination.

FIG. 12 is a schematic diagram of the study design used in Apo E −/− mice.

FIGS. 13A and 13B are graphs showing the plasma lipid levels after treatment with a control or an Apo E mimetic. A) cholesterol; B) triglycerides. Each graph shows the lipid levels after the treatment has been withdrawn for four weeks (control and hE18A) and the lipid levels immediately after four weeks of treatment (control Term. 1 and hE18A Term. 1).

FIG. 14 is a bar graph showing the liver cholesteryl ester and free cholesterol.

FIG. 15 is a diagram of the aorta showing the regions that were analyzed for plaques or lesions.

FIG. 16 is a bar graph of percent plaque in the aorta versus the different treatments. “Term 1” represents the results after the initial four weeks of treatment. “Term 2” represents the results after treatment has been withdrawn for four weeks.

FIG. 17 is a bar graph showing plaque area in the aortic arch at week 5 and week 8.

FIGS. 18A and 18B are graphs showing the diastolic (A) and the systolic (B) area.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

### A. Definitions

The terminology used herein is for the purpose of describing particular embodiments only and is not intended to be limiting.

As used in the specification and the appended claims, the singular forms “a,” “an” and “the” can include plural referents unless the context clearly dictates otherwise. Thus, for example, reference to “a compound” includes mixtures of compounds, reference to “a pharmaceutical carrier” includes mixtures of two or more such carriers, and the like.

Ranges may be expressed herein as from “about” one particular value, and/or to “about” another particular value. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

As used herein, the term “amino acid sequence” refers to a list of abbreviations, letters, characters or words representing amino acid residues. The amino acid abbreviations used herein are conventional one letter codes for the amino acids and are expressed as follows: A, alanine; C, cysteine; D aspartic acid; E, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine.

As used herein, the term “Apo E mimetic” is interchangeable with apolipoprotein-E mimicking peptide. Apo E mimetics are peptides that are related to, characteristic of, or mimic Apo E. Apo E mimetics include Apo E peptides (i.e. peptides derived from full length Apo E).

“Dosing regimen” as used herein refers to at least one treatment cycle followed by at least one rest phase. A dosing regimen can include more than one treatment cycle and more than one rest phase. For example, a dosing regimen can be a three month treatment cycle followed by a one year rest phase. Another example can be a six month treatment cycle followed by a six month rest phase and then a three month treatment cycle followed by a one year rest phase.

As used herein, the term “treatment cycle” refers to the administration of Apo E mimetics for an established period of time. A treatment cycle includes a wide range of dosages of Apo E mimetics as well as different lengths of time for administering the Apo E mimetics. For example, a treatment cycle can be a three month period wherein an Apo E mimetic is administered twice a week for the three month period.

“Dose” or “dosage” as used herein refers to a specific quantity of a therapeutic agent, such as an Apo E mimetic, that is taken at specific times.

As used herein, “effective amount” is meant to mean a sufficient amount of the composition or Apo E mimetic to provide the desired effect. For example, an effective amount of an Apo E mimetic can be an amount that provides a therapeutic affect and provides sustained therapeutic effects after withdrawal of the treatment. An effective amount of an Apo E mimetic is an amount that is able to cause a benefit illustrated by a decrease in atherosclerosis, a decrease in artery wall stiffness, a decrease in isolated systolic hypertension, a decrease in arterial inflammation, an increase in anti-oxidant capability of the HDL fraction and/or an improvement in myocardial function, as well as an amount that allows for a sustained therapeutic effect after withdrawal of the Apo E mimetic. The exact amount required will vary from subject to subject, depending on the species, age, and general condition of the subject, the severity of disease (or underlying genetic defect) that is being treated, the particular compound used, its mode of administration, and the like. Thus, it is not possible to specify an exact “effective amount.” However, an appropriate “effective amount” may be determined by one of ordinary skill in the art using only routine experimentation.

As used herein, “sustained therapeutic effect” is a therapeutic effect that persists after the therapeutic has been withdrawn. For example, the sustained therapeutic effect is maintained even after the acute cholesterol lowering effect is gone.

“Rest phase” as used herein refers to a period of time wherein an Apo E mimetic is not administered.

Atherosclerotic burden as used herein is the amount of atherosclerosis in the arteries of a patient. This may include the coronary, carotid, peripheral and other arteries. The atheroma may be complex lesions with a smooth muscle and collagen containing fibrous cap, areas of calcification, cholesterol crystals and cholesterol laden macrophages (foam cells) and/or less complex and more unstable lesions with less calcification and a thinner fibrous cap, and more foam cells and cholesterol (unstable lesions). The unstable lesions may intrude into the lumen of the artery or expand away from the lumen of the artery.

“Peptide” as used herein refers to any peptide, oligopeptide, polypeptide, gene product, expression product, or protein. A peptide is comprised of consecutive amino acids. The term “peptide” encompasses naturally occurring or synthetic molecules.

The word “or” as used herein means any one member of a particular list and also includes any combination of members of that list.

As used herein, “reverse oriented”, “reversed orientation”, “reverse analog” or “reverse sequence” refers to a peptide, or a portion of the peptide, has a reverse amino acid sequence as compared to a non-reverse oriented peptide (i.e., the original sequence is read (or written) from right to left). For example, if one peptide has the amino acid sequence ABCDE, its reverse analog or a peptide having its reverse sequence is as follows: EDCBA. In a dual domain peptide for example, Ac-hE-18A-NH2, either the hE sequence is read from right to left or the 18A sequence is read from right to left. For a reverse analog of, LRKLRKRLLR-DWLKAFYDKVAEKLKEAF (SEQ ID NO:1) can be RLLRKRLKRL-DWLKAFYDKVAEKLKEAF (SEQ ID NO:2) or LRKLRKRLLR-FAEKLKEAVKDYFAKLWD (SEQ ID NO:3).

As used herein a “dual-domain peptide”, a “dual-domain synthetic peptide”, or a “dual-domain Apo E mimicking peptide” is meant to mean a peptide comprising a lipid-associating peptide/domain and a receptor binding peptide/domain.

As used herein a “single-domain peptide”, a “single-domain synthetic peptide”, or a “single-domain Apo E mimicking peptide” is meant to mean a peptide comprising either a lipid-associating peptide/domain or a receptor binding peptide/domain, but not both.

As used herein “domain switched”, “switched domain”, or “switched” peptide is meant to mean that the lipid-associating peptide is covalently linked to the receptor binding domain of apolipoprotein E such that the lipid-associating peptide is at the N-terminus of the synthetic apolipoprotein E-mimicking peptide. For example, the peptide 18A-hE is exemplary of a domain switched peptide.

As used herein, “scrambled” “scrambled version”, or “scrambled peptide” is meant to mean that the composition of the amino acid sequence is the same as the unscrambled peptide, however the sequence of the amino acids is altered thus rendering the peptide unable to form either an α-amphipathic helix or does not possess lipid associating (or HSPG associating) properties. However, in some cases, as described in this invention, the scrambled peptide remains able to form a different helical structure, such as a π-helix. For example, if one peptide has the amino acid sequence ABCDE, the scrambled version of the peptide could have the amino acid sequence DEABC. Scrambled peptides are often denoted as having a “Sc” prior to the portion of the peptide that is scrambled. For example, Sc-hE-18A denoted that the hE portion of the peptide is scrambled.

As used herein, “sample” is meant to mean an animal; a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

As used herein, “subject” refers to the target of administration, e.g. an animal. Thus the subject of the disclosed methods can be a vertebrate, such as a mammal. For example, the subject can be a human. The term does not denote a particular age or sex. Subject can be used interchangeably with “individual” or “patient”.

As used herein, “modulate” is meant to mean to alter, by increasing or decreasing.

As used herein “lipid binding domain E” and “lipid-associating peptide” are used interchangeably. As used herein, both terms can mean the lipid binding domain of apolipoprotein E.

As used herein, “isolated polypeptide” or “purified polypeptide” is meant to mean a polypeptide (or a fragment thereof) that is substantially free from the materials with which the polypeptide is normally associated in nature. The polypeptides of the invention, or fragments thereof, can be obtained, for example, by extraction from a natural source (for example, a mammalian cell), by expression of a recombinant nucleic acid encoding the polypeptide (for example, in a cell or in a cell-free translation system), or by chemically synthesizing the polypeptide. In addition, polypeptide fragments may be obtained by any of these methods, or by cleaving full length proteins and/or polypeptides.

As used herein, “treat” is meant to mean administer one of the disclosed compositions to a subject, such as a human or other mammal (for example, an animal model), that has atherosclerosis, in order to prevent or delay a worsening of the effects of the disease or condition, or to partially or fully reverse the effects of the disease.

As used herein, “prevent” is meant to mean minimize the chance that a subject who has an increased susceptibility for developing atherosclerosis will develop atherosclerosis.

As used herein, “lipoprotein” or “lipoproteins” is meant to mean a biochemical assembly that contains both proteins and lipids. The lipids or their derivatives may be covalently or non-covalently bound to the proteins. Many enzymes, transporters, structural proteins, antigens, adhesins, and toxins are lipoproteins. Examples include the high density and low density lipoproteins of the blood, the transmembrane proteins of the mitochondrion and the chloroplast, and bacterial lipoproteins

As used herein, “high-density lipoprotein” (HDL) is meant to mean a class of lipoproteins, varying somewhat in their size (8-11 nm in diameter), that can transport cholesterol. HDL cholesterol is cholesterol that is associated with HDLs. About one-fourth to one-third of blood cholesterol is carried by high-density lipoprotein (HDL). HDL cholesterol is known as “good” cholesterol, because high levels of HDL seem to protect against heart attack. Low levels of HDL (less than 40 mg/dL in men and less than 50 mg/dL in women) also increase the risk of heart disease. Medical experts think that HDL tends to carry cholesterol away from the arteries and back to the liver, where it is passed from the body. Some experts believe that that HDL removes excess cholesterol from arterial plaque, thus slowing its buildup

As used herein, “very Low Density Lipoproteins” (VLDL) is meant to mean a lipoprotein subclass. It is assembled in the liver from cholesterol and apolipoproteins. It is converted in the bloodstream to low density lipoprotein (LDL). VLDL particles have a diameter of 30-80 nm. VLDL transports endogenous products where chylomicrons transport exogenous (dietary) products.

As used herein, “low-density lipoprotein” or “LDL” is mean to mean a lipoprotein that varies in size (approx. 22 nm) and can contain a changing number of triglycerides and cholesteryl esters they actually have a mass and size distribution. Each native LDL particle contains a single apolipoproteinB-100 molecule (Apo B-100, a protein with 4536 amino acid amino acid residues) and a phospholipid coat that circles the triglycerides and cholesteryl esters, keeping them soluble in the aqueous environment. LDL is commonly referred to as bad cholesterol. LDL cholesterol is cholesterol that is associated with LDLs. When too much LDL cholesterol circulates in the blood, it can slowly build up in the inner walls of the arteries that feed the heart and brain. Together with other substances, it can form plaque, a thick, hard deposit that can narrow the arteries and make them less flexible. This condition is known as atherosclerosis. If a clot forms and blocks a narrowed artery, then heart attack or stroke can result.

Cholesterol cannot dissolve in the blood. It has to be transported to and from the cells by carriers called lipoproteins. LDLs and HDLs along with triglyceride-rich lipoproteins (VLDL) and Lp(a) cholesterol, make up your total cholesterol count, which can be determined through a blood test.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

### B. Dosing Regimens

Disclosed herein are dosing regimens comprising at least one treatment cycle of an effective amount of an Apo E mimetic followed by a rest phase. The rest phase of the dosing regimen is a period of time where the Apo E mimetic is not administered.

Disclosed herein are dosing regimens comprising at least one treatment cycle followed by a rest phase, wherein the treatment cycle comprises administering an effective amount of an Apo E mimetic to allow for a sustained therapeutic effect after withdrawal of the Apo E mimetic, wherein the Apo E mimetic is not administered during the rest phase. Not only does an effective amount of Apo E mimetic result in sustained therapeutic effects, but it is also an amount sufficient to cause an acute beneficial effect. Thus, the effects of the Apo E mimetic can be measured and seen during the treatment cycle, at the end of the treatment cycle and during the rest phase. The sustained therapeutic effects are the therapeutic effects seen even after the acute cholesterol lowering effect is gone.

Disclosed herein are dosing regimens comprising at least one treatment cycle followed by a rest phase, wherein the treatment cycle comprises administering an effective amount of an Apo E mimetic to allow for a sustained therapeutic effect after withdrawal of the Apo E mimetic, wherein the Apo E mimetic is not administered during the rest phase, wherein the treatment cycle comprises administration of an effective amount of the Apo E mimetic once a week for three months or wherein the treatment cycle comprises administration of an effective amount of the Apo E mimetic once every two weeks for up to 12 weeks.

Disclosed herein are dosing regimens comprising at least one treatment cycle followed by a rest phase, wherein the treatment cycle comprises administering an effective amount of an Apo E mimetic to allow for a sustained therapeutic effect after withdrawal of the Apo E mimetic, wherein the Apo E mimetic is not administered during the rest phase, wherein the dosing regimen further comprises a second treatment cycle after the rest phase.

Disclosed herein are dosing regimens comprising at least one treatment cycle followed by a rest phase, wherein the treatment cycle comprises administering an effective amount of an Apo E mimetic to allow for a sustained therapeutic effect after withdrawal of the Apo E mimetic, wherein the ApoE mimetic consists of the amino acid sequence LRKLRKRLLRDWLKAFYDKVAEKLKEAF (SEQ ID NO:1), wherein the Apo E mimetic is not administered during the rest phase. In some instances, the amino acid sequence LRKLRKRLLRDWLKAFYDKVAEKLKEAF (SEQ ID NO:1), is end protected with an acetyl group protecting the amino terminus and an amide group protecting the carboxyl terminus.

The dosing regimen can further include a second treatment cycle after the rest phase. A second rest phase can occur after the second treatment cycle. In some instances a third, fourth, fifth, sixth, seventh, eighth, ninth or tenth treatment cycle can be administered wherein each treatment cycle is followed by a rest phase. In one aspect, the dosing regimen includes infinite treatment cycles, each followed by a rest phase. For example, a subject may be prescribed a dosing regimen that involves consecutive treatment cycles followed by rest phases for the duration of their life.

In one aspect, a second dosing regimen can be prescribed based on the re-occurrence of atherosclerotic lesions or other atherosclerosis factors. The second dosing regimen can be administered 1, 2, 3, 4, 5 years or more than 5 years after the initial dosing regimen was administered. The second dosing regimen can be the same as the initial dosing regimen or can be different. For example, the initial dosing regimen can be a three month treatment cycle followed by a one year rest phase. After the one year rest phase the subject can be tested and if atherosclerotic lesions are building up again then a second dosing regimen consisting of another three month treatment cycle followed by a rest phase or a six month treatment cycle followed by a rest phase can be prescribed. The dose of Apo E mimetic can vary between the initial dosing regimen and any additionally prescribed dosing regimens.

1. Treatment Cycle

The treatment cycle can include the administration of different dosages of Apo E mimetic as well as administration at different time points. The Apo E mimetic can be administered for varying amounts of time for up to 6 months. In some instances, the administration can occur for up to one, two, three, four, five or six months. For example, the Apo E mimetic can be administered once a week for 4, 6, 8, 10, 12, 14, 16, 18, 20, 22, or 24 weeks.

The length of time for each treatment cycle can vary depending on the amount of Apo E administered per dosage. A treatment cycle can include the administration of Apo E mimetic once, twice or three times a week. In some aspects, the Apo E mimetic can be administered daily. In some aspects, the Apo E mimetic can be administered once every two weeks or even once a month. In some instances, the Apo E mimetic can be administered every two weeks for 4, 6, 8, 10, 12, 14, 16, 18, 20, 22, or 24 weeks. For example, the treatment cycle can include administering an Apo E mimetic once a week for four weeks or once every two weeks for up to six months. Thus, each treatment cycle includes an established length of time for administration as well as an established dosing schedule during that time frame.

In one aspect, more than one Apo E mimetic can be administered during the treatment cycles. The more than one Apo E mimetic can be formulated together or in separate compositions. In some instances, one or more Apo E mimetic is administered in combination with one or more other therapeutic agents, such as cholesterol lowering drugs.

2. Rest Phase

The disclosed dosing regimen includes at least one treatment cycle followed by a rest phase. The rest phase is a period of time wherein Apo E mimetic is not administered and the length of the period of time can vary. The length of the rest phase is dependent on how long the sustained therapeutic effects of the Apo E mimetic administered during the treatment cycle last. In some instances the rest phase can be at least 1, 2, 3, 4, 5, 6, 7, 8, 9, 10, 11, or 12 months. In some instances the rest phase can be at least 1, 2, 3, 4, 5, 6, 7, 8, 9, or 10 years. For example, the rest phase can be at least four weeks (one month).

One way to determine how long the rest phase should last is to test the subject to determine the progression of the atherosclerosis burden in the subject's arteries. If the atherosclerosis burden has progressed to a level that increases the risk of cardiovascular disease, then the subject can be prescribed a second dosing regimen. If the atherosclerosis burden is stable then the rest phase can be prolonged. Subjects can be tested on a regular basis. For example, a subject can be tested every 3, 6, 9, 12, 18, 24, 30 or 36 months.

In one aspect, the rest phase can be decreased or extended depending on the dose of Apo E mimetic administered and the reduction in atherosclerosis achieved during the treatment cycle. For example, the rest phase can be extended if the dose of Apo E mimetic during the treatment cycle is increased and the atherosclerosis burden is substantially reduced. The length of the rest phase can also vary based on the length of the treatment cycle. For instance, if a subject receives a certain dose of Apo E mimetic once a week for three months then the rest phase may be shorter than a subject that receives the same dose of Apo E mimetic once a week for six months.

Although an Apo E mimetic is not administered during the rest phase, an atherosclerosis therapeutic other than an Apo E mimetic can be administered during the rest phase. The atherosclerosis therapeutic other than an Apo E mimetic can be a conventional lipid lowering therapy, such as a statin, a bile acid sequestrant or a fibrate, or a novel anti-atherosclerosis therapeutic like a CETP inhibitor, a VLDL synthesis inhibitor, a PCSK9 inhibitor, and/or an arterial inflammation inhibitor.

In some instances, the beneficial effects of the Apo E mimetic can still be present in a subject even after the treatment cycle is complete. In one instance, the half-life of the Apo E mimetic is less than 1, 2, 3, 4, 5, 10, 15, 20, 25, or 30 days. In some instances the Apo E mimetic is no longer detectable in a subject after the treatment cycle is complete. Thus, the long-term therapeutic effects are not from residual Apo E mimetic.

3. Dose

The dose or dosage of Apo E mimetic can vary depending on many factors, such as but not limited to, age, condition, sex and extent of the disease in the patient, route of administration, length of treatment cycle, or whether other drugs are included in the regimen, and can be determined by one of skill in the art.

Effective dosages can be determined empirically, and making such determinations is within the skill in the art. The dosage ranges for the administration of the compositions are those large enough to produce the desired effect in which the disease is treated. For example, the dosage can be an amount effective to provide therapeutic effects and provide or allow for sustained therapeutic effects even after the treatment (i.e. Apo E mimetic) is withdrawn. The therapeutic effects can be, but are not limited to, a reduction in atherosclerotic lesions, decrease in arterial stiffness, decrease in isolated systolic hypertension, increase in vasoresponsiveness, improvement in cardiac function, increased blood flow to the limbs, decreased claudication, increased ankle brachial pressure index, improved healing of distal limb wounds. The therapeutic effects can be measured by markers of arterial inflammation such as, but not limited to, C-reactive protein. The therapeutic effects can be measured by atherosclerosis imaging techniques, including MRI, intravascular ultrasound, ultrafast imaging CT scans, B-mode ultrasonography, virtual histology intravascular ultrasound, optical coherence tomography, or other known methods.

The dosage should not be so large as to cause adverse side effects, such as unwanted cross-reactions, anaphylactic reactions, and the like. The dosage can be adjusted by the individual physician in the event of any counter-indications. Dosage can vary, and can be administered in one or more dose administrations daily, for one or several days. Guidance can be found in the literature for appropriate dosages for given classes of pharmaceutical products.

Suitable dosages include, but are not limited to amounts between 0.01 mg/kg and 10 mg/kg. For example, disclosed herein are methods involving administering one or more of the disclosed Apo E mimetics to a subject, wherein the Apo E mimetic is administered in an amount of about 0.15 mg/kg to about 5 mg/kg.

The Apo E mimetic dose can be administered as a bolus injection or as an infusion over one or more hours.

4. Apo E Mimetics

Disclosed are apolipoprotein-E mimicking peptides or Apo E mimetics. Non-limiting examples of the Apo E mimetics are provided herein. The Apo E mimetics can be single domain or dual domain peptides. Compositions containing the Apo E mimetics are also disclosed.

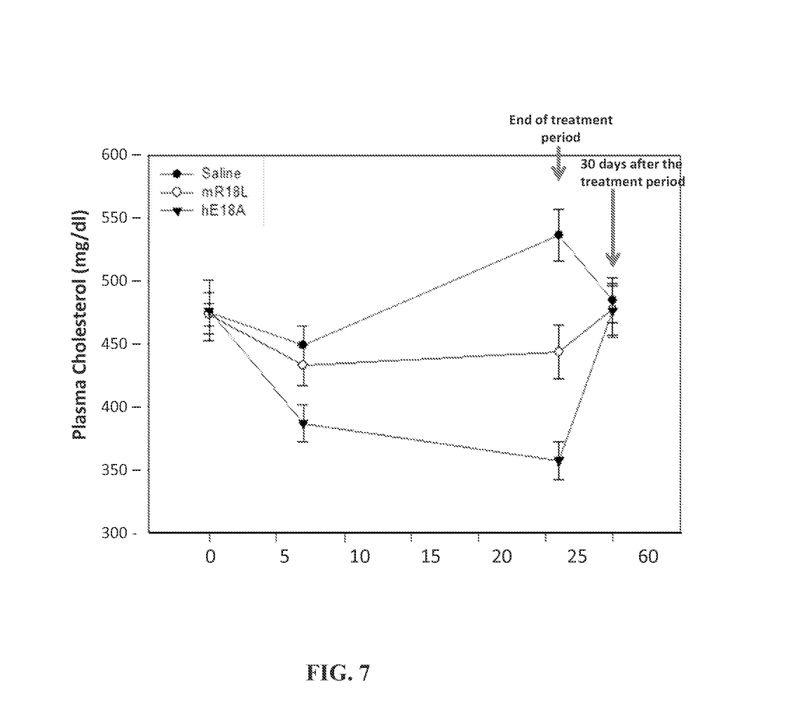

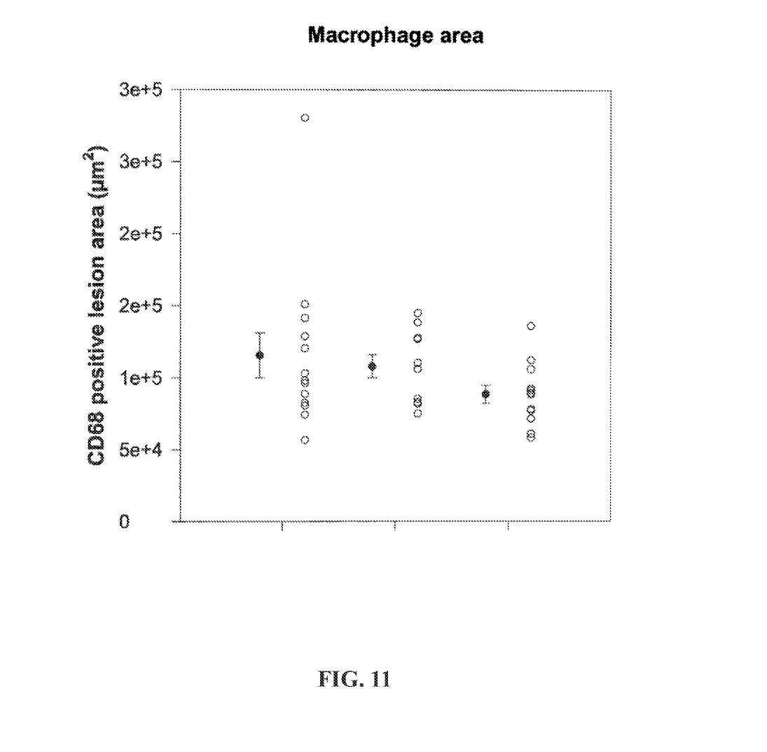

Apolipoprotein E mimetics have both direct cholesterol lowering effects by providing an alternative ligand for receptors on the liver to clear atherogenic Apolipoprotein B containing lipoproteins (LDL, VDLD and β-VLDL), and direct beneficial effects on the artery wall. New, more effective methods of imaging coronary atherosclerosis allow for direct measurement of benefits to the artery wall (Van Velzen, et al. Hellenic J Cardiol 50: 245-263, 2009). The Apo E mimetics can enhance the removal of cholesterol from the artery wall, working in conjunction with HDL, increasing the formation of lipid poor preβ-HDL that accept cholesterol from macrophages. The Apo E mimetics can stimulate macrophage-mediated clearance of dead and dying cells in the artery wall (efferocytosis), improve the quality of HDL by increasing PON-1 levels and bringing down plasma lipid hydroperoxide levels, decrease macrophage content in atherosclerotic lesions resulting in more stable lesions, and decrease inflammation in the artery wall. As a result, the Apo E mimetics reduce the size of atherosclerotic lesions more rapidly and to a far greater extent than the statins (HMG-CoA reductase inhibitors). FIGS. 7 and 11 show that atherosclerotic lesion regression persists in Apo E mimetic treated animals even when cholesterol levels are the same as in saline treated animals. Thus, the effects cannot be simply explained by cholesterol lowering.

i. Apolipoprotein E

Apolipoprotein E (Apo E) plays an important role in the metabolism of triglyceride-rich lipoproteins, such as very low density lipoprotein (VLDL) and chylomicrons. Apolipoprotein E mediates the high affinity binding of Apo E-containing lipoproteins to the low density lipoprotein (LDL) receptor (Apo B, E receptor) and the members of its gene family, including LDL receptor related protein (LRP), very low density lipoprotein receptor (VLDLR) and the Apo E2 receptor (Apo E2R) (Mahley, R. W., (1988) Science 240, 622-630). The putative and complex role of Apo E in atherosclerosis has been emphasized by several observations: (i) mice that over express human Apo E have lower levels of total plasma cholesterol levels (Shimono, H. N., et al., (1992) Eur. J. Clin. Invest. 90, 2084-2991), (ii) intravenous injection of human Apo E into cholesterol-fed rabbits protects these animals from atherosclerosis (Yamada, et al., (1989) Proc. Natl. Acad. Sci. U.S.A. 86, 665-669), and (iii) loss of the Apo E gene in mice produces spontaneous atherosclerosis (Zhang, S. H., et al., (1992) Science 258, 468-471) which is ameliorated when macrophage-specific apo E expression is initiated in Apo E-deficient mice (Spangenberg, J., et al., (1997) Biochem. Biophys. Acta 1349, 109-121).

Apo E is a protein that binds lipid and has two major domains (Mahley, R. W., et al. J. Lipid Res. 1999, 40:622-630). The 22 kDa amino terminal domain has been shown by X-ray crystallographic studies to be a 4-helix bundle (Wilson, C., et al. Science 1991; 252:1817-1822) and to contain a positively-charged receptor binding domain. For this region to mediate very low-density lipoprotein (VLDL) binding to its receptors, the apolipoprotein must associate with the lipoprotein surface; this is enabled by the C-terminal amphipathic helical region. If the 4-helix bundle that contains the positively charged receptor-binding domain does not open up on the lipoprotein surface, then the VLDL is defective in binding to receptors. Thus, the positively charged arginine (Arg)-rich cluster domain of the Apo E and the C-terminal amphipathic helical domain, are both required for the enhanced uptake of atherogenic Apo E-containing lipoproteins.

Apo E is secreted as a 299 amino acid residue protein with a molecular weight of 34,200. Based on thrombin cleavage of Apo E into two fragments, a two-domain hypothesis was initially suggested to explain the fact that the C-terminal region of Apo E (192-299) is essential for its binding to hypertriglyceridemic VLDL and the N-terminal 22 kDa domain (1-191), binds to the LDL-R (Bradley, W. A., et al., (1986) J. Lipid Res. 27, 40-48). Additional physical-chemical characterization of the protein and its mutants have extended this concept and have shown that the region 192-211 binds to phospholipid while the amino terminal domain (1-191) is a globular structure that contains the LDL receptor binding domain in the 4-helix bundle (Wilson, C., et al., (1991) Science 252, 1817-1822). Studies with synthetic peptides (Sparrow et al. Biochemistry 31(4):1065-8, 1992) and monoclonal antibodies pinpointed the LDL receptor binding domain of apo E between residues 129-169, a domain enriched in positively charged amino acids, Arg and Lys (Rall, S. C., Jr., et al., (1982) PNAS USA 79, 4696-4700; Lalazar, A., et al., (1988) J. Biol. Chem. 263, 3542-2545; Dyer, C. A., et al., (1991) J. Biol. Chem. 296, 22803-22806; and Dyer, C. A., et al., (1991) J. Biol. Chem. 266, 15009-15015).

To test the hypothesis that a minimal arginine-rich Apo E receptor binding domain (141-150) was sufficient to enhance low density lipoprotein (LDL) and very low density lipoprotein (VLDL) uptake and clearance when covalently linked to a class A amphipathic helix, a peptide was synthesized in which the receptor binding domain of human Apo E, LRKLRKRLLR (SEQ ID NO:4) (hApo E[141-150] also referred to as “hE”), was linked to 18A, a well characterized high affinity lipid-associating peptide (DWLKAFYDKVAEKLKEAF (SEQ ID NO:5), also referred to as “18A”) to produce a peptide denoted as hApo E[141-150]-18A (also referred to as “hE-18A”) (see U.S. Pat. No. 6,506,880, which is hereby incorporated by reference in its entirety for its teaching of specific Apo E mimetics and their uses). Also synthesized was an end protected analog of hE-18A, denoted Ac-hE18A-NH2. The importance of the lysine residues and the role of the hydrophobic residues in the receptor binding domain were also studied using two analogs, LRRLRRRLLR-18A (SEQ ID NO:6) (also referred to as “hE(R)-18A”) and LRKMRKRLMR-18A (SEQ ID NO:7) (also referred to as “mE18A”), whereby the receptor binding domain of human Apo E was modified to substitute arginine (R) residues for lysine (K) residues at positions 143 and 146 (LRRLRRRLLR; SEQ ID NO:6) and whereby the receptor binding domain of mouse Apo E (LRKMRKRLMR; SEQ ID NO:7), were linked to 18A, respectively. The effect of the dual character peptides on the uptake and degradation of human LDL/VLDL by cells was then determined.

It was determined that in MEF 1 cells with induced LDL receptors, LDL internalization was enhanced three, five and seven times by Ac-mE-18A-NH2, Ac-hE-18A-NH2, and Ac-hE(R)-18A-NH2 respectively. All three peptides increased degradation of LDL by 100 percent. Both Ac-hE-18A-NH2 and the control peptide Ac-18A-NH2 interacted with VLDL to cause a displacement of apo E from VLDL. However, only Ac-hE-18A-NH2-associated VLDL enhanced the uptake of VLDL six fold and degradation three fold compared to VLDL alone in spite of the absence of apoE. The LDL binding to fibroblasts in the presence of these peptides was not saturable, however, over the LDL concentration range studied.

Furthermore, a similar enhancement of LDL internalization independent of the presence of the LDL receptor related protein (LRP) or LDL receptor or both was seen. Pretreatment of cells with heparinase and heparitinase however abolished greater than 80% of enhanced peptide-mediated LDL uptake and degradation by cells. The data indicated that the dual-domain peptides enhanced LDL uptake and degradation by binding to the LDL through the amphipathic lipid binding domain (18A). However, the minimal 141-150 Arg-rich domain did not decrease LDL levels but did so only in combination with 18A lipid associating domain, did not confer LDL-receptor binding but directed the LDL-peptide complex to the HSPG pathway for uptake and degradation by fibroblasts.

ii. Single Domain Peptides

Disclosed are single-domain synthetic Apo E mimetics. The single-domain synthetic Apo E mimetics can consist of a receptor binding domain of Apo E or a lipid-associating peptide.

a. Receptor Binding Domain Peptides

The receptor binding domain peptide for the synthetic Apo E mimetics can be a human receptor binding domain peptide of Apo E. For example, receptor binding domain peptide of the disclosed synthetic Apo E mimetics can comprise the amino acid sequence of LRKLRKRLLR (SEQ ID NO:4), LRRLRRRLLR (SEQ ID NO:6), or LRKLRKRFFR (SEQ ID NO:7). The receptor binding domain peptide of such synthetic Apo E mimetics can also be from a species selected from the group consisting of mouse, rabbit, monkey, rat, bovine, pig and dog.

Examples of receptor binding domain peptides that can be used in the disclosed synthetic Apo E mimetics are provided in Table 1.

TABLE 1
Disclosed Synthetic Apo E mimetics

| Species | Starting Residue NO: | Sequence |
|---|---|---|
| Human | 141 | LRKLRKRLLR |
| | | (SEQ ID NO: 4) |
| Rabbit | 134 | LRKLRKRLLR |
| | | (SEQ ID NO: 4) |
| Monkey | 141 | LRKLRKRLLR |
| | | (SEQ ID NO: 4) |
| Mouse | 133 | LRKMRKRLMR |
| | | (SEQ ID NO: 7) |
| Rat | 133 | LRKMRKRLMR |
| | | (SEQ ID NO: 7) |
| Bovine | 140 | LRKL [symbol] KRLLR |
| | | (SEQ ID NO: 8) |
| Pig | 140 | LRNVRKRLVR |
| | | (SEQ ID NO: 9) |
| Dog | 133 | MRKLRKRVLR |
| | | (SEQ ID NO: 10) |
| R Modified | 141 | LRRLRRRLLR |
| | | (SEQ ID NO: 11) |
| F Modified | 141 | LRKLRKR [symbol] R |
| | | (SEQ ID NO: 12) |
| ApoB | | [symbol] |
| | | (SEQ ID NO: 13) |

The italicized residues in Table 1 indicate changes from the human sequence; however, the property of the amino acid is conserved. The bold-italicized residues in Table 1 indicate the difference from the human sequence at that position.

The receptor binding domain peptide for the synthetic Apo E mimetics can also be the LDL receptor (LDLR) binding domain of apolipoprotein B (ApoB). The LDL receptor (LDLR) binding domain of ApoB can have the sequence RLTRKRGLK (SEQ ID NO:13). ApoB-100 is a 550,000 Da glycoprotein with nine amino acids (3359-3367) serving as the binding domain for the LDL receptor (Segrest et al., J. Lipid. Res. 42, pp. 1346-1367 (2001)). Upon binding to LDLR in clathrin coated pits, LDL is internalized via endocytosis and moves into the endosome where a drop in pH causes the receptor to dissociate from the LDL. The receptor is recycled back to the surface of the cell while the LDL is moved into the lysosome where the particle is degraded (Goldstein et al., Ann. Rev. Cell Biol. 1, pp. 1-39 (1985)). The LDL receptor (LDLR) binding domain of ApoB when used with the disclosed peptides can also be altered and/or modified as described throughout this application for Apo E. For example, LDL receptor (LDLR) binding domain of ApoB can be used with the disclosed lipid-associating peptides, wherein the LDL receptor (LDLR) binding domain of ApoB is covalently linked to said lipid-associating peptide. In addition, the LDL receptor (LDLR) binding domain of ApoB can be scrambled, reverse-oriented, can be part of a domain switched peptide as described below.

b. Lipid-Associating Peptides

Lipid-associating peptides can be used alone or in combination with the Apo E-mimicking peptides. The lipid associating peptide for these synthetic Apo E mimetics can be, but are not limited to, class A amphipathic helical peptides, class A amphipathic helical peptide mimetics of apoA-I having aromatic or aliphatic residues in the non-polar face, small peptides including pentapeptides, tetrapeptides, tripeptides, dipeptides and pairs of amino acids, Apo-J (G* peptides), and peptide mimetics, e.g., as described below.

(A) Class A Amphipathic Helical Peptides

In one aspect, the lipid-associating peptides for use in the disclosed methods include class A amphipathic helical peptides, e.g. as described in U.S. Pat. No. 6,664,230, and PCT Publications WO 02/15923 and WO 2004/034977. It was discovered that peptides comprising a class A amphipathic helix (“class A peptides”), are capable of mitigating one or more symptoms of atherosclerosis as well as treating other disorders.

Class A peptides are characterized by formation of an α-helix that produces a segregation of polar and non-polar residues thereby forming a polar and a nonpolar face with the positively charged residues residing at the polar-nonpolar interface and the negatively charged residues residing at the center of the polar face (see, e.g., Anantharamaiah (1986) Meth. Enzymol, 128: 626-668). It is noted that the fourth exon of apo A-I, when folded into 3.667 residues/turn produces a class A amphipathic helical structure.

One class A peptide, designated 18A (see, e.g., Anantharamaiah (1986) Meth. Enzymol, 128: 626-668) was modified as described herein to produce peptides orally administrable and highly effective at inhibiting or preventing one or more symptoms of atherosclerosis and/or other indications described herein. Without being bound by a particular theory, it is believed that the disclosed peptides can act in vivo by picking up seeding molecule(s) that mitigate oxidation of LDL.

Increasing the number of Phe residues on the hydrophobic face of 18A can increase lipid affinity as determined by the computation described by Palgunachari et al. (1996) Arteriosclerosis, Thrombosis, & Vascular Biol. 16: 328-338. Theoretically, a systematic substitution of residues in the nonpolar face of 18A with Phe could yield six peptides. Peptides with an additional 2, 3 and 4 Phe would have theoretical lipid affinity (λ) values of 13, 14 and 15 units, respectively. However, the λ values jumped four units if the additional Phe were increased from 4 to 5 (to 19λ units). Increasing to 6 or 7 Phe would produce a less dramatic increase (to 20 and 21λ units, respectively).

A number of these class A peptides were made including, the peptide designated 4F, D4F, 5F, and D5F, and the like. Various class A peptides inhibited lesion development in atherosclerosis-susceptible mice. In addition, the peptides show varying, but significant degrees of efficacy in mitigating one or more symptoms of the various pathologies described herein. A number of such peptides are illustrated in Table 2.

TABLE 2
Class A peptides

| Peptide | |
|---|---|
| Name | Amino Acid Sequence |
| 18F | D-W-L-K-A-F-Y-D-K-V-A-E-K-L-K-E-A-F |
| | (SEQ ID NO: 5) |
| 2F | Ac-D-W-L-K-A-F-Y-D-K-V-A-E-K-L-K-E-A- |
| | F-NH₂ (SEQ ID NO: 5) |
| 3F | Ac-D-W-F-K-A-F-Y-D-K-V-A-E-K-L-K-E-A- |
| | F-NH₂ (SEQ ID NO: 14) |
| 3F14 | Ac-D-W-L-K-A-F-Y-D-K-V-A-E-K-F-K-E-A- |
| | F-NH₂ (SEQ ID NO: 15) |
| 4F | Ac-D-W-F-K-A-F-Y-D-K-V-A-E-K-F-K-E-A- |
| | F-NH₂ (SEQ ID NO: 16) |
| 5F | Ac-D-W-L-K-A-F-Y-D-K-V-F-E-K-F-K-E-F-F- |
| | NH₂ (SEQ ID NO: 17) |
| 6F | Ac-D-W-L-K-A-F-Y-D-K-F-F-E-K-F-K-E-F-F- |
| | NH₂ (SEQ ID NO: 18) |
| 7F | Ac-D-W-F-K-A-F-Y-D-K-F-F-E-K-F-K-E-F-F- |
| | NH₂ (SEQ ID NO: 19) |
| | Ac-D-W-L-K-A-F-Y-D-K-V-A-E-K-L-K-E-F-F- |
| | NH₂ (SEQ ID NO: 20) |
| | Ac-D-W-L-K-A-F-Y-D-K-V-F-E-K-F-K-E-A-F- |
| | NH₂ (SEQ ID NO: 21) |
| | Ac-D-W-L-K-A-F-Y-D-K-V-F-E-K-L-K-E-F-F- |
| | NH₂ (SEQ ID NO: 22) |
| | Ac-D-W-L-K-A-F-Y-D-K-V-A-E-K-F-K-E-F-F- |
| | NH₂ (SEQ ID NO: 23) |
| | Ac-D-W-L-K-A-F-Y-D-K-V-F-E-K-F-K-E-F-F- |
| | NH₂ (SEQ ID NO: 24) |
| | Ac-E-W-L-K-L-F-Y-E-K-V-L-E-K-F-K-E-A-F- |
| | NH₂ (SEQ ID NO: 25) |
| | Ac-E-W-L-K-A-F-Y-ID-K-V-A-E-K-F-K-E-A-F- |
| | NH₂ (SEQ ID NO: 26) |
| | Ac-E-W-L-K-A-F-Y-D-K-V-A-E-K-L-K-E-F-F- |
| | NH₂ (SEQ ID NO: 27) |
| | Ac-E-W-L-K-A-F-Y-D-K-V-F-E-K-F-K-E-A-F- |
| | NH₂ (SEQ ID NO: 28) |
| | Ac-E-W-L-K-A-F-Y-D-K-V-F-E-K-L-K-E-F-F- |
| | NH₂ (SEQ ID NO: 29) |
| | Ac-E-W-L-K-A-F-Y-D-K-V-A-E-K-F-K-E-F-F- |
| | NH₂ (SEQ ID NO: 30) |
| | Ac-E-W-L-K-A-F-Y-D-K-V-F-E-K-F-K-E-F-F- |
| | NH₂ (SEQ ID NO: 31) |
| | Ac-A-F-Y-D-K-V-A-E-K-L-K-E-A-F-NH₂ |
| | (SEQ ID NO: 32) |
| | Ac-A-F-Y-D-K-V-A-E-K-F-K-E-A-F-NH₂ (SEQ |
| | ID NO: 33) |
| | Ac-A-F-Y-D-K-V-A-E-K-F-K-E-A-F-NH₂ |
| | (SEQ ID NO: 34) |
| | Ac-A-F-Y-D-K-F-F-E-K-F-K-E-F-F-NH₂ (SEQ |
| | ID NO: 35) |
| | Ac-A-F-Y-D-K-F-F-E-K-F-K-E-F-F-NH₂ (SEQ |
| | ID NO: 36) |
| | Ac-A-F-Y-D-K-V-A-E-K-F-K-E-A-F-NH₂ (SEQ |
| | ID NO: 37) |
| | Ac-A-F-Y-D-K-V-A-E-K-L-K-E-F-F-NH₂ (SEQ |
| | ID NO: 38) |
| | Ac-A-F-Y-D-K-V-F-E-K-F-K-E-A-F-NH₂ (SEQ |
| | ID NO: 39) |
| | Ac-A-F-Y-D-K-V-F-E-K-L-K-E-F-F-NH₂ (SEQ |
| | ID NO: 40) |
| | Ac-A-F-Y-D-K-V-A-E-K-F-K-E-F-F-NH₂ |
| | (SEQ ID NO: 41) |
| | Ac-K-A-F-Y-D-K-V-F-E-K-F-K-E-F-NH₂ |
| | (SEQ ID NO: 42) |
| | Ac-L-F-Y-E-K-V-L-E-K-F-K-E-A-F-NH₂ |
| | (SEQ ID NO: 43) |
| | Ac-A-F-Y-D-K-V-A-E-K-F-K-E-A-F-NH₂ |
| | (SEQ ID NO: 44) |
| | Ac-A-F-Y-D-K-V-A-E-K-L-K-E-F-F-NH₂ |
| | (SEQ ID NO: 45) |
| | Ac-A-F-Y-D-K-V-F-E-K-F-K-E-A-F-NH₂ |
| | (SEQ ID NO: 46) |
| | Ac-A-F-Y-D-K-V-F-E-K-L-K-E-F-F-NH₂ |
| | (SEQ ID NO: 47) |
| | Ac-A-F-Y-D-K-V-A-E-K-F-K-E-F-F-NH₂ |
| | (SEQ ID NO: 48) |
| | Ac-A-F-Y-D-K-V-F-E-K-F-K-E-F-F NH₂ |
| | (SEQ ID NO: 49) |
| | Ac-D-W-L-K-A-L-Y-D-K-V-A-E-K-L-K-E- |
| | A-L-NH₂ (SEQ ID NO: 50) |
| | Ac-D-W-F-K-A-F-Y-E-K-V-A-E-K-L-K-E- |
| | F-F-NH₂ (SEQ ID NO: 51) |
| | Ac-D-W-F-K-A-F-Y-E-K-F-F-E-K-F-K-E-F- |
| | F-NH₂ (SEQ ID NO: 52) |
| | Ac-E-W-L-K-A-L-Y-E-K-V-A-E-K-L-K-E- |
| | A-L-NH₂ (SEQ ID NO: 53) |
| | Ac-E-W-L-K-A-F-Y-E-K-V-A-E-K-L-K-E- |
| | A-F-NH₂ (SEQ ID NO: 54) |
| | Ac-E-W-F-K-A-F-Y-E-K-V-A-E-K-L-K-E- |
| | F-F-NH₂ (SEQ ID NO: 55) |
| | Ac-E-W-L-K-A-F-Y-E-K-V-F-E-K-F-K-E-F- |
| | F-NH₂ (SEQ ID NO: 56) |
| | Ac-E-W-L-K-A-F-Y-E-K-F-F-E-K-F-K-E-F- |
| | F-NH₂ (SEQ ID NO: 57) |
| | Ac-E-W-F-K-A-F-Y-E-K-F-F-E-K-F-K-E-F- |
| | F-NH₂ (SEQ ID NO: 58) |
| | Ac-D-F-L-K-A-W-Y-D-K-V-A-E-K-L-K-E-A- |
| | W-NH₂ (SEQ ID NO: 59) |
| | Ac-E-F-L-K-A-W-Y-E-K-V-A-E-K-L-K-E-A- |
| | W-NH₂ (SEQ ID NO: 60) |
| | Ac-D-F-W-K-A-W-Y-D-K-V-A-E-K-L-K-E- |
| | W-W-NH₂ (SEQ ID NO: 61) |
| | Ac-E-F-W-K-A-W-Y-E-K-V-A-E-K-L-K-E- |
| | W-W-NH₂ (SEQ ID NO: 62) |
| | Ac-D-K-L-K-A-F-Y-D-K-V-F-E-W-A-K-E-A- |
| | F-NH₂ (SEQ ID NO: 63) |
| | Ac-D-K-W-K-A-V-Y-D-K-F-A-E-A-F-K-E-F- |
| | L-NH₂ (SEQ ID NO: 64) |
| | Ac-E-K-L-K-A-F-Y-E-K-V-F-E-W-A-K-E-A- |
| | F-NH₂ (SEQ ID NO: 65) |
| | Ac-E-K-W-K-A-V-Y-E-K-F-A-E-A-F-K-E-F- |
| | L-NH₂ (SEQ ID NO: 66) |
| | Ac-D-W-L-K-A-F-V-D-K-F-A-E-K-F-K-E-A- |
| | Y-NH₂ (SEQ ID NO: 67) |
| | Ac-E-K-W-K-A-V-Y-E-K-F-A-E-A-F-K-E-F- |
| | L-NH₂ (SEQ ID NO: 68) |
| | Ac-D-W-L-K-A-F-V-Y-D-K-V-F-K-L-K-E- |
| | F-F-NH₂ (SEQ ID NO: 69) |
| | Ac-E-W-L-K-A-F-V-Y-E-K-V-F-K-L-K-E-F-F- |
| | NH₂ (SEQ ID NO: 70) |
| | Ac-D-W-L-R-A-F-Y-D-K-V-A-E-K-L-K-E-A-F- |
| | NH₂ (SEQ ID NO: 71) |
| | Ac-E-W-L-R-A-F-Y-E-K-V-A-E-K-L-K-E-A-F- |
| | NH₂ (SEQ ID NO: 72) |
| | Ac-D-W-L-K-A-F-Y-D-R-V-A-E-K-L-K-E-A-F- |
| | NH₂ (SEQ ID NO: 73) |
| | Ac-E-W-L-K-A-F-Y-E-R-V-A-E-K-L-K-E-A-F- |
| | NH₂ (SEQ ID NO: 74) |
| | Ac-D-W-L-K-A-F-Y-D-K-V-A-E-R-L-K-E-A-F- |
| | NH₂ (SEQ ID NO: 75) |
| | Ac-E-W-L-K-A-F-Y-E-K-V-A-E-R-L-K-E-A-F- |
| | NH₂ (SEQ ID NO: 76) |
| | Ac-D-W-L-K-A-F-Y-D-K-V-A-E-K-L-R-E-A- |
| | F-NH₂ (SEQ ID NO: 77) |
| | Ac-E-W-L-K-A-F-Y-E-K-V-A-E-K-L-R-E-A-F- |
| | NH₂ (SEQ ID NO: 78) |
| | Ac-D-W-L-K-A-F-Y-D-R-V-A-E-R-L-K-E-A-F- |
| | NH₂ (SEQ ID NO: 79) |
| | Ac-E-W-L-K-A-F-Y-E-R-V-A-E-R-L-K-E-A-F- |
| | NH₂ (SEQ ID NO: 80) |
| | Ac-D-W-L-R-A-F-Y-D-K-V-A-E-K-L-R-E-A-F- |
| | NH₂ (SEQ ID NO: 81) |
| | Ac-E-W-L-R-A-F-Y-E-K-V-A-E-K-L-R-E-A-F- |
| | NH₂ (SEQ ID NO: 82) |
| | Ac-D-W-L-R-A-F-Y-D-R-V-A-E-K-L-K-E-A- |
| | F-NH₂ (SEQ ID NO: 83) |
| | Ac-E-W-L-R-A-F-Y-E-R-V-A-E-K-L-K-E-A- |
| | F-NH₂ (SEQ ID NO: 84) |
| | Ac-D-W-L-K-A-F-Y-D-K-V-A-E-R-L-R-E-A- |
| | F-NH₂ (SEQ ID NO: 85) |
| | Ac-E-W-L-K-A-F-Y-E-K-V-A-E-R-L-R-E-A- |
| | F-NH₂ (SEQ ID NO: 86) |
| | Ac-D-W-L-R-A-F-Y-D-K-V-A-E-R-L-K-E-A- |
| | F-NH₂ (SEQ ID NO: 87) |
| | Ac-E-W-L-R-A-F-Y-E-K-V-A-E-R-L-K-E-A- |
| | F-NH₂ (SEQ ID NO: 88) |
| | D-W-L-K-A-F-Y-D-K-V-A-E-K-L-K-E-A-F-P- |
| | D-W |
| | L-K-A-F-Y-D-K-V-A-E-K-L-K-E-A-F (SEQ ID |
| | NO: 89) |
| | D-W-L-K-A-F-Y-D-K-V-A-E-K-L-K-E-F-F-P- |
| | D-W |
| | L-K-A-F-Y-D-K-V-A-E-K-L-K-E-F-F (SEQ |
| | ID NO: 90) |
| | D-W-F-K-A-F-Y-D-K-V-A-E-K-L-K-E-A-F-P- |
| | D-W |
| | F-K-A-F-Y-D-K-V-A-E-K-L-K-E-A-F (SEQ |
| | ID NO: 91) |
| | D-K-L-K-A-F-Y-D-K-V-F-E-W-A-K-E-A-F-P- |
| | D-K |
| | L-K-A-F-Y-D-K-V-F-E-W-L-K-E-A-F (SEQ |
| | ID NO: 92) |
| | D-K-W-K-A-V-Y-D-K-F-A-E-A-F-K-E-F-L-P- |
| | D-K |
| | W-K-A-V-Y-D-K-F-A-E-A-F-K-E-F-L (SEQ |
| | ID NO: 93) |
| | D-W-F-K-A-F-Y-D-K-V-A-E-K-F-K-E-A-F-P- |
| | D-W |
| | F-K-A-F-Y-D-K-V-A-E-K-F-K-E-A-F (SEQ |
| | ID NO: 94) |
| | D-W-L-K-A-F-V-Y-D-K-V-F-K-L-K-E-F-F-P- |
| | D-W |
| | L-K-A-F-V-Y-D-K-V-F-K-L-K-E-F-F (SEQ ID |
| | NO: 95) |
| | D-W-L-K-A-F-Y-D-K-F-A-E-K-F-K-E-F-F-P- |
| | D-W |
| | L-K-A-F-Y-D-K-F-A-E-K-F-K-E-F-F (SEQ ID |
| | NO: 96) |
| | Ac-E-W-F-K-A-F-Y-E-K-V-A-E-K-F-K-E-A- |
| | F-NH₂ (SEQ ID NO: 97) |
| | Ac-D-W-F-K-A-F-Y-D-K-V-A-E-K-F-NH₂ |
| | (SEQ ID NO: 98) |
| | Ac-F-K-A-F-Y-D-K-V-A-E-K-F-K-E-NH₂ |
| | (SEQ ID NO: 99) |
| | Ac-F-K-A-F-Y-E-K-V-A-E-K-F-K-E-NH₂ |
| | (SEQ ID NO: 100) |
| | NMA-F-K-A-F-Y-D-K-V-A-E-K-F-K-E-NH₂ |
| | (SEQ ID NO: 101) |
| | NMA-F-K-A-F-Y-E-K-V-A-E-K-F-K-E-NH₂ |
| | (SEQ ID NO: 102) |
| | NMA-D-W-F-K-A-F-Y-D-K-V-A-E-K-F-K-E- |
| | A-F-NH₂ (SEQ ID NO: 103) |
| | NMA-E-W-F-K-A-F-Y-E-K-V-A-E-K-F-K-E- |
| | A-F-NH₂ (SEQ ID NO: 104) |
| | NMA-A-F-Y-D-K-V-A-E-K-F-K-E-A-F-NH₂ |
| | (SEQ ID NO: 105) |
| | NMA-D-W-F-K-A-F-Y-D-K-V-A-E-K-F-NH₂ |
| | (SEQ ID NO: 106) |
| | Ac-D-W-L-K-A-F-Y-D-K-V-F-E-K-F-K-E-F-F- |
| | NH₂ (SEQ ID NO: 107) |
| | NMA-D-W-L-K-A-F-Y-D-K-V-F-E-K-F-K-E-F- |
| | F-NH₂ (SEQ ID NO: 108) |
| | Ac-E-W-L-K-A-F-Y-E-K-V-F-E-K-F-K-E-F-F- |
| | NH₂ (SEQ ID NO: 109) |
| | NMA-E-W-L-K-A-F-Y-E-K-V-F-E-K-F-K-E-F- |
| | F-NH₂ (SEQ ID NO: 110) |
| | Ac-A-F-Y-D-K-V-F-E-K-F-K-E-F-F-NH₂ (SEQ |
| | ID NO: 111) |
| | NMA-A-F-Y-D-K-V-F-E-K-F-K-E-F-F-NH₂ |
| | (SEQ ID NO: 112) |
| | Ac-A-F-Y-E-K-V-F-E-K-F-K-E-F-F-NH₂ (SEQ |
| | ID NO: 113) |
| | NMA-A-F-Y-E-K-V-F-E-K-F-K-E-F-F-NH₂ |
| | (SEQ ID NO: 114) |
| | Ac-D-W-L-K-A-F-Y-D-K-V-F-E-K-F-NH₂ |
| | (SEQ ID NO: 115) |
| | NMA-D-W-L-K-A-F-Y-D-K-V-F-E-K-F-NH₂ |
| | (SEQ ID NO: 116) |
| | Ac-E-W-L-K-A-F-Y-E-K-V-F-E-K-F-NH₂ (SEQ |
| | ID NO: 117) |
| | NMA-E-W-L-K-A-F-Y-E-K-V-F-E-K-F-NH₂ |
| | (SEQ ID NO: 118) |
| | Ac-L-K-A-F-Y-D-K-V-F-E-K-F-K-E-NH₂ (SEQ |
| | ID NO: 119) |
| | NMA-L-K-A-F-Y-D-K-V-F-E-K-F-K-E-NH₂ |
| | (SEQ ID NO: 120) |
| | Ac-L-K-A-F-Y-E-K-V-F-E-K-F-K-E-NH₂ (SEQ |
| | ID NO: 121) |
| | NMA-L-K-A-F-Y-E-K-V-F-E-K-F-K-E-NH₂ |
| | (SEQ ID NO: 122) |

*Linkers are underlined.
NMA is N-Methyl Anthranilyl

In certain aspects, the peptides include variations of 4F (D-W-K-A-F-Y-D-K-V-A-E-K-F-K-E-A-F (SEQ ID NO:16) in Table 2), also known as L-4F, where all residues are L form amino acids) or D-4F where one or more residues are D form amino acids). In any of the peptides described herein, the C-terminus, and/or N-terminus, and/or internal residues can be blocked with one or more blocking groups as described herein.

While various peptides of Table 2, are illustrated with an acetyl group or an N-methylanthranilyl group protecting the amino terminus and an amide group protecting the carboxyl terminus, any of these protecting groups may be eliminated and/or substituted with another protecting group as described herein. The peptides can comprise one or more D-form amino acids as described herein. In certain aspects, every amino acid (e.g., every enantiomeric amino acid) of the peptides of Table 2 is a D-form amino acid.

It is also noted that Table 2 is not fully inclusive. Using the teachings provided herein, other suitable class A amphipathic helical peptides can routinely be produced (e.g., by conservative or semi-conservative substitutions (e.g., D replaced by E), extensions, deletions, and the like). Thus, for example, one embodiment utilizes truncations of any one or more of peptides shown herein (e.g., peptides identified as 2F, 3F, 3F14, 4F, 5F, 6F, or 7F—in Table 2). Thus, for example, A-F-Y-D-K-V-A-E-K-L-K-E-A-F (amino acids 5-18 of SEQ ID NO:5) illustrates a peptide comprising 14 amino acids from the C-terminus of 18A comprising one or more D amino acids, while others illustrate other truncations. Longer peptides are also suitable. Such longer peptides may entirely form a class A amphipathic helix, or the class A amphipathic helix (helices) can form one or more domains of the peptide. In addition, this invention contemplates multimeric versions of the peptides (e.g., concatamers). Thus, for example, the peptides illustrated herein can be coupled together (directly or through a linker (e.g., a carbon linker, or one or more amino acids) with one or more intervening amino acids). Illustrative polymeric peptides include 18A-Pro-18A and the peptides in the following table, in certain embodiments comprising one or more D amino acids, more preferably with every amino acid a D amino acid as described herein and/or having one or both termini protected.

(SEQ ID NO: 90)

D-W-L-K-A-F-Y-D-K-V-A-E-K-L-K-E-A-F-P-
D-W
L-K-A-F-Y-D-K-V-A-E-K-L-K-E-A-F

(SEQ ID NO: 91)

D-W-L-K-A-F-Y-D-K-V-A-E-K-L-K-E-F-F-P-
D-W
L-K-A-F-Y-D-K-V-A-E-K-L-K-E-F-F
D-W-F-K-A-F-Y-D-K-V-A-E-K-L-K-E-A-F-P-
D-W

(SEQ ID NO: 92)

F-K-A-F-Y-D-K-V-A-E-K-L-K-E-A-F
D-K-L-K-A-F-Y-D-K-V-F-E-W-A-K-E-A-F-P-
D-K

(SEQ ID NO: 93)

L-K-A-F-Y-D-K-V-F-E-W-L-K-E-A-F
D-K-W-K-A-V-Y-D-K-F-A-E-A-F-K-E-F-L-P-
D-K

(SEQ ID NO: 94)

W-K-A-V-Y-D-K-F-A-E-A-F-K-E-F-L
D-W-F-K-A-F-Y-D-K-V-A-E-K-F-K-E-A-F-P-
D-W

(SEQ ID NO: 95)

F-K-A-F-Y-D-K-V-A-E-K-F-K-E-A-F
D-W-L-K-A-F-V-Y-D-K-V-F-K-L-K-E-F-F-P-
D-W

(SEQ ID NO: 96)

L-K-A-F-V-Y-D-K-V-F-K-L-K-E-F-F
D-W-L-K-A-F-Y-D-K-F-A-E-K-F-K-E-F-F-P-
D-W

(SEQ ID NO: 97)

L-K-A-F-Y-D-K-F-A-E-K-F-K-E-F-F

(B) Class a Amphipathic Helical Peptide Mimetics of apoA-I Having Aromatic or Aliphatic Residues in the Non-Polar Face.

Also disclosed are modified class A amphipathic helix peptides. Certain preferred peptides incorporate one or more aromatic residues at the center of the nonpolar face, e.g., 3F^{Cπ}, (as present in 4F), or with one or more aliphatic residues at the center of the nonpolar face, e.g., 3F^{Iπ}, see, e.g., Table 3. Without being bound to a particular theory, the central aromatic residues on the nonpolar face of the peptide 3F^{Cπ}, due to the presence of π electrons at the center of the nonpolar face can allow water molecules to penetrate near the hydrophobic lipid alkyl chains of the peptide-lipid complex, which in turn would enable the entry of reactive oxygen species (such as lipid hydroperoxides) shielding them from the cell surface. The peptides with aliphatic residues at the center of the nonpolar face, e.g., 3F^{Iπ}, can act similarly but not quite as effectively as 3F^{Cπ}.

In one aspect, the peptides can convert pro-inflammatory HDL to anti-inflammatory HDL or make anti-inflammatory HDL more anti-inflammatory, and/or decrease LDL-induced monocyte chemotactic activity generated by artery wall cells equal to or greater than D4F or other peptides shown in Table 2.

TABLE 3
Modified class A peptides

| Name | Sequence |
|---|---|
| (3F^{cn}) | Ac-DKWKAVYDKFAEAFKEFL-NH₂ |
| | (SEQ ID NO: 123) |
| (3F¹ⁿ) | Ac-DKLKAFYDKVFEWAKEAF-NH₂ |
| | (SEQ ID NO: 124) |

(C) Other Class A and Some Class Y Amphipathic Helical Peptides.

Class A amphipathic helical peptides that have an amino acid composition identical to one or more of the class A amphipathic helical peptides described above. Thus, for example, in certain embodiments this invention contemplates peptides having an amino acid composition identical to 4F. Thus, in certain embodiments, this invention includes active agents that comprise a peptide that consists of 18 amino acids, where the 18 amino acids consist of 3 alanines (A), 2 aspartates (D), 2 glutamates (E), 4 phenylalanines (F), 4 lysines (K), 1 valine (V), 1 tryptophan (W), and 1 tyrosine (Y); and where the peptide forms a class A amphipathic helix; and protects a phospholipid against oxidation by an oxidizing agent. In various embodiments, the peptides comprise least one “D” amino acid residue; and in certain embodiments, the peptides comprise all “D: form amino acid residues. A variety of such peptides are illustrated in Table 4. Reverse (retro-), inverse, retro-inverso-, and circularly permuted forms of these peptides are also contemplated.

TABLE 4
Illustrative 18 amino acid length class A amphipathic
helical peptides with the amino acid composition
3 alanines (A), 2 aspartates (D), 2 glutamates (E),
4 phenylalanines (F), 4 lysines (K), 1 valine (V),
1 tryptophan (W), and 1 tyrosine (Y).

| Name | Sequence |
|---|---|
| [Switch D-E]-4F analogs | |
| [Switch D-E]-1-4F | Ac-EWFKAFYEKVADKFKDAF-NH₂ |
| | (SEQ ID NO: 125) |
| [Switch D-E]-2-4F | Ac-EWFKAFYDKVADKFKEAF-NH₂ |
| | (SEQ ID NO: 126) |
| [Switch D-E]-3-4F | Ac-DWFKAFYEKVADKFKEAF-NH₂ |
| | (SEQ ID NO: 127) |
| [Switch D-E]-4-4F | Ac-DWFKAFYEKVAEKFKDAF-NH₂ |
| | (SEQ ID NO: 128) |
| [W-2, F-3 positions reversed] | |
| 4F-2 | Ac-DFWKAFYDKVAEKFKEAF-NH₂ |
| | (SEQ ID NO: 129) |
| [Switch D-E]-1-4F-2 | Ac-EFWKAFYEKVADKFKDAF-NH₂ |
| | (SEQ ID NO: 130) |
| [Switch D-E]-2-4F-2 | Ac-EFWKAFYDKVADKFKEAF-NH₂ |
| | (SEQ ID NO: 131) |
| [Switch D-E]-3-4F-2 | Ac-DFWKAFYEKVADKFKEAF-NH₂ |
| | (SEQ ID NO: 132) |
| [Switch D-E]-4-4F-2 | Ac-DFWKAFYEKVAEKFKDAF-NH₂ |
| | (SEQ ID NO: 133) |
| [F-6 and Y-7 positions switched] | |
| 4F-3 | Ac-DWFKAYFDKVAEKFKEAF-NH₂ |
| | (SEQ ID NO: 134) |
| [Switch D-E]-1-4F-5 | Ac-EWFKAYFEKVADKFKDAF-NH₂ |
| | (SEQ ID NO: 135) |
| [Switch D-E]-2-4F-5 | Ac-EWFKAYFDKVADKFKEAF-NH₂ |
| | (SEQ ID NO: 136) |
| [Switch D-E]-3-4F-5 | Ac-DWFKAYFEKVADKFKEAF-NH₂ |
| | (SEQ ID NO: 137) |
| [Switch D-E]-4-4F-5 | Ac-DWFKAYFEKVAEKFKDAF-NH₂ |
| | (SEQ ID NO: 138) |
| [Y-land 10V positions switched] | |
| 4F-4 | Ac-DWFKAFVDKYAEKFKEAF-NH₂ |
| | (SEQ ID NO: 139) |
| [Switch D-E]-1-4F-4 | Ac-EWFKAFVEKYADKFKDAF-NH₂ |
| | (SEQ ID NO: 140) |
| [Switch D-E]-2-4F-4 | Ac-EWFKAFVDKYADKFKEAF-NH₂ |
| | (SEQ ID NO: 141) |
| [Switch D-E]-3-4F-4 | Ac-DWFKAFVEKYADKFKEAF-NH₂ |
| | (SEQ ID NO: 142) |
| [Switch D-E]-4-4F | Ac-DWFKAFVEKYAEKFKDAF-NH₂ |
| | (SEQ ID NO: 143) |
| [V-10 and A-11 switched] | |
| 4-F-5 | Ac-DWFKAFYDKAVEKFKEAF-NH₂ |
| | (SEQ ID NO: 144) |
| [Switch D-E]-1-4F-5 | Ac-EWFKAFYEKAVDKFKDAF-NH₂ |
| | (SEQ ID NO: 145) |
| [Switch D-E]-2-4F-5 | Ac-EWFKAFYDKAVDKFKEAF-NH₂ |
| | (SEQ ID NO: 146) |
| [Switch D-E]-3-4F-5 | Ac-DWFKAFYEKAVDKFKEAF-NH₂ |
| | (SEQ ID NO: 147) |
| [Switch D-E]-4-4F-5 | Ac-DWFKAFYEKAVEKFKDAF-NH₂ |
| | (SEQ ID NO: 148) |
| [A-11 and F-14 switched] | |
| 4F-6 | Ac-DWFKAFYDKVFEKAKEAF-NH₂ |
| | (SEQ ID NO: 149) |
| [Switch D-E]-1-4F-6 | Ac-EWFKAFYEKVFDKAKDAF-NH₂ |
| | (SEQ ID NO: 150) |
| [Switch D-E]-2-4F-6 | Ac-EWFKAFYDKVFDKAKEAF-NH₂ |
| | (SEQ ID NO: 151) |
| [Switch D-E]-3-4F-6 | Ac-DWFKAFYEKVFDKAKEAF-NH₂ |
| | (SEQ ID NO: 152) |
| [Switch D-E]-4-4F-6 | Ac-DWFKAFYEKVFEKAKDAF-NH₂ |
| | (SEQ ID NO: 153) |
| [F-14 and A-17 switched] | |
| 4F-7 | Ac-DWFKAFYDKVAEKAKEFF-NH₂ |
| | (SEQ ID NO: 154) |
| [Switch D-E]-1-4F-7 | Ac-EWFKAFYEKVADKAKDFF-NH₂ |
| | (SEQ ID NO: 155) |
| [Switch D-E]-2-4F-7 | Ac-EWFKAFYDKVADKAKEFF-NH₂ |
| | (SEQ ID NO: 156) |
| [Switch D-E]-3-4F-7 | Ac-DWFKAFYEKVADKAKEFF-NH₂ |
| | (SEQ ID NO: 157) |
| [Switch D-E]-4-4F-7 | Ac-DWFKAFYEKVAEKAKDFF-NH₂ |
| | (SEQ ID NO: 158) |
| [A-17 and F-18 switched] | |
| 4F-8 | Ac-DWFKAFYDKVAEKFKEFA-NH₂ |
| | (SEQ ID NO: 159) |
| [Switch D-E]-1-4F-8 | Ac-EWFKAFYEKVADKFKDFA-NH₂ |
| | (SEQ ID NO: 160) |
| [Switch D-E]-2-4F-8 | Ac-EWFKAFYDKVADKFKEFA-NH₂ |
| | (SEQ ID NO: 161) |
| [Switch D-E]-3-4F-8 | Ac-DWFKAFYEKVADKFKEFA-NH₂ |
| | (SEQ ID NO: 162) |
| [Switch D-E]-4-4F-8 | Ac-DWFKAFYEKVAEKFKDFA-NH₂ |
| | (SEQ ID NO: 163) |
| [W-2 and A-17 switched] | |
| 4F-9 | Ac-DAFKAFYDKVAEKFKEWF-NH₂ |
| | (SEQ ID NO: 164) |
| [Switch D-E]-1-4F-9 | Ac-EAFKAFYEKVADKFKDWF-NH₂ |
| | (SEQ ID NO: 165) |
| [Switch D-E]-2-4F-9 | Ac-EAFKAFYDKVADKFKEWF-NH₂ |
| | (SEQ ID NO: 166) |
| [Switch D-E]-3-4F-9 | Ac-DAFKAFYEKVADKFKEWF-NH₂ |
| | (SEQ ID NO: 167) |
| [Switch D-E]-4-4F-9 | Ac-DAFKAFYEKVAEKFKDWF-NH₂ |
| | (SEQ ID NO: 168) |
| [W-2 and A-11 switched] | |
| 4F-10 | Ac-DAFKAFYDKVWEKFKEAF-NH₂ |
| | (SEQ ID NO: 169) |
| [Switch D-E]-1-4F-10 | Ac-EAFKAFYEKVWDKFKDAF-NH₂ |
| | (SEQ ID NO: 170) |
| [Switch D-E]-2-4F-10 | Ac-EAFKAFYDKVWDKFKEAF-NH₂ |
| | (SEQ ID NO: 171) |
| [Switch D-E]-3-4F-10 | Ac-DAFKAFYEKVWDKFKEAF-NH₂ |
| | (SEQ ID NO: 172) |
| [Switch D-E]-4-4F-10 | Ac-DAFKAFYEKVWEKFKDAF-NH₂ |
| | (SEQ ID NO: 173) |
| [W-2 and Y-7 switched] | |
| 4F-11 | Ac-DYFKAFWDKVAEKFKEAF-NH₂ |
| | (SEQ ID NO: 174) |
| [Switch D-E]-1-4F-11 | Ac-EYFKAFWEKVADKFKDAF-NH₂ |
| | (SEQ ID NO: 175) |
| [Switch D-E]-2-4F-11 | Ac-EYFKAFWDKVADKFKEAF-NH₂ |
| | (SEQ ID NO: 176) |
| [Switch D-E]-3-4F-11 | Ac-DYFKAFWEKVADKFKEAF-NH₂ |
| | (SEQ ID NO: 177) |
| [Switch D-E]-4-4F-11 | Ac-DYFKAFWEKVAEKFKDAF-NH₂ |
| | (SEQ ID NO: 178) |
| [F-3 and A-17 switched] | |
| 4F-12 | Ac-DWAKAFYDKVAEKFKEFF-NH₂ |
| | (SEQ ID NO: 179) |
| [Switch D-E]-1-4F-12 | Ac-EWAKAFYEKVADKFKDFF-NH₂ |
| | (SEQ ID NO: 180) |
| [Switch D-E]-2-4F-12 | Ac-EWAKAFYDKVADKFKEFF-NH₂ |
| | (SEQ ID NO: 181) |
| [Switch D-E]-3-4F-12 | Ac-DWAKAFYEKVADKFKEFF-NH₂ |
| | (SEQ ID NO: 182) |
| [Switch D-E]-4-4F-12 | Ac-DWAKAFYEKVAEKFKDFF-NH₂ |
| | (SEQ ID NO: 183) |
| [F-6 and A-17 switched] | |
| 4F-13 | Ac-DWFKAAYDKVAEKFKEFF-NH₂ |
| | (SEQ ID NO: 184) |
| [Switch D-E]-1-4F-13 | Ac-EWFKAAYEKVADKFKDFF-NH₂ |
| | (SEQ ID NO: 185) |
| [Switch D-E]-2-4F-13 | Ac-EWFKAAYDKVADKFKEFF-NH₂ |
| | (SEQ ID NO: 186) |
| [Switch D-E]-3-4F-13 | Ac-DWFKAAYEKVADKFKEFF-NH₂ |
| | (SEQ ID NO: 187) |
| [Switch D-E]-4-4F-13 | Ac-DWFKAAYEKVAEKFKDFF-NH₂ |
| | (SEQ ID NO: 188) |
| [Y-7 and A-17 switched | |
| 4F-14 | Ac-DWFKAFADKVAEKFKEYF-NH₂ |
| | (SEQ ID NO: 189) |
| [Switch D-E]-1-4F-14 | Ac-EWFKAFAEKVADKFKDYF-NH₂ |
| | (SEQ ID NO: 190) |
| [Switch D-E]-2-4F-14 | Ac-EWFKAFADKVADKFKEYF-NH₂ |
| | (SEQ ID NO: 191) |
| [Switch D-E]-3-4F-14 | Ac-DWFKAFAEKVADKFKEYF-NH₂ |
| | (SEQ ID NO: 192) |
| [Switch D-E]-4-4F | Ac-DWFKAFAEKVAEKFKDYF-NH₂ |
| | (SEQ ID NO: 193) |
| [V-10 and A-17 switched] | |
| 4F-15 | Ac-DWFKAFYDKAAEKFKEVF-NH₂ |
| | (SEQ ID NO: 194) |
| [Switch D-E]-1-4F-15 | Ac-EWFKAFYEKAADKFKDVF-NH₂ |
| | (SEQ ID NO: 195) |
| [Switch D-E]-2-4F-15 | Ac-EWFKAFYDKAADKFKEVF-NH₂ |
| | (SEQ ID NO: 196) |
| [Switch D-E]-3-4F-15 | Ac-DWFKAFYEKAADKFKEVF-NH₂ |
| | (SEQ ID NO: 197) |
| [Switch D-E]-4-4F-15 | Ac-DWFKAFYEKAAEKFKDVF-NH₂ |
| | (SEQ ID NO: 198) |
| [F3 and Y-7 switched] | |
| 4F-16 | Ac-DWYKAFFDKVAEKFKEAF-NH₂ |
| | (SEQ ID NO: 199) |
| [Switch D-E]-1-4F-16 | Ac-EWYKAFFEKVADKFKDAF-NH₂ |
| | (SEQ ID NO: 200) |
| [Switch D-E]-2-4F-16 | Ac-EWYKAFFDKVADKFKEAF-NH₂ |
| | (SEQ ID NO: 201) |
| [Switch D-E]-3-4F-16 | Ac-DWYKAFFEKVADKFKEAF-NH₂ |
| | (SEQ ID NO: 202) |
| [Switch D-E]-4-4F-16 | Ac-DWYKAFFEKVAEKFKDAF-NH₂ |
| | (SEQ ID NO: 203) |
| [F-3 and V-10 switched] | |
| 4F-17 | Ac-DWVKAFYDKFAEKFKEAF-NH₂ |
| | (SEQ ID NO: 204) |
| [Switch D-E]-1-4F-17 | Ac-EWVKAFYEKFADKFKDAF-NH₂ |
| | (SEQ ID NO: 205) |
| [Switch D-E]-2-4F-17 | Ac-EWVKAFYDKFADKFKEAF-NH₂ |
| | (SEQ ID NO: 206) |
| [Switch D-E]-3-4F-17 | Ac-DWVKAFYEKFADKFKEAF-NH₂ |
| | (SEQ ID NO: 207) |
| [Switch D-E]-4-4F-17 | Ac-DWVKAFYEKFAEKFKDAF-NH₂ |
| | (SEQ ID NO: 208) |
| [Y-7 and F-14 switched] | |
| 4F-18 | Ac-DWFKAFFDKVAEKYKEAF-NH₂ |
| | (SEQ ID NO: 209) |
| [Switch D-E]-1-4F-18 | Ac-EWFKAFFEKVADKYKDAF-NH₂ |
| | (SEQ ID NO: 210) |
| [Switch D-E]-2-4F-18 | Ac-EWFKAFFDKVADKYKEAF-NH₂ |
| | (SEQ ID NO: 211) |
| [Switch D-E]-3-4F-18 | Ac-DWFKAFFEKVADKYKEAF-NH₂ |
| | (SEQ ID NO: 212) |
| [Switch D-E]-3-4F-18 | Ac-DWFKAFFEKVADKYKEAF-NH₂ |
| | (SEQ ID NO: 213) |
| [Y-7 and F-18 switched] | |
| 4F-19 | Ac-DWFKAFFDKVAEKFKEAY-NH₂ |
| | (SEQ ID NO: 214) |
| [Switch D-E]-1-4F-19 | Ac-EWFKAFFEKVADKFKDAY-NH₂ |
| | (SEQ ID NO: 215) |
| [Switch D-E]-2-4F-19 | Ac-EWFKAFFDKVADKFKEAY-NH₂ |
| | (SEQ ID NO: 216) |
| [Switch D-E]-3-4F-19 | Ac-DWFKAFFEKVADKFKEAY-NH₂ |
| | (SEQ ID NO: 217) |
| [Switch D-E]-4-4F-19 | Ac-DWFKAFFEKVAEKFKDAY-NH₂ |
| | (SEQ ID NO: 218) |
| [V-10 and F-18 switched] | |
| 4F-20 | Ac-DWFKAFYDKFAEKFKEAV-NH₂ |
| | (SEQ ID NO: 219) |
| [Switch D-E]-1-4F-20 | Ac-EWFKAFYEKFADKFKDAV-NH₂ |
| | (SEQ ID NO: 220) |
| [Switch D-E]-2-4F-20 | Ac-EWFKAFYDKFADKFKEAV-NH₂ |
| | (SEQ ID NO: 221) |
| [Switch D-E]-3-4F-20 | Ac-DWFKAFYEKFADKFKEAV-NH₂ |
| | (SEQ ID NO: 222) |
| [Switch D-E]-4-4F-20 | Ac-DWFKAFYEKFAEKFKDAV-NH₂ |
| | (SEQ ID NO: 223) |
| [W-2 and K13 switched] | |
| 4F-21 | Ac-DKFKAFYDKVAEKFWEAF-NH₂ |
| | (SEQ ID NO: 224) |
| [Switch D-E]-1-4F-21 | Ac-EKFKAFYEKVADKFWDAF-NH₂ |
| | (SEQ ID NO: 225) |
| [Switch D-E]-2-4F-21 | Ac-EKFKAFYDKVADKFWEAF-NH₂ |
| | (SEQ ID NO: 226) |
| [Switch D-E]-3-4F-21 | Ac-DKFKAFYEKVADKFWEAF-NH₂ |
| | (SEQ ID NO: 227) |
| [Switch D-E]-4-4F-21 | Ac-DKFKAFYEKVAEKFWDAF-NH₂ |
| | (SEQ ID NO: 228) |
| [W-3, F-13 and K-2 4F] | |
| 4F-22 | Ac-DKWKAFYDKVAEKFFEAF-NH₂ |
| | (SEQ ID NO: 229) |
| [Switch D-E]-1-4F-22 | Ac-EKWKAFYEKVADKFFDAF-NH₂ |
| | (SEQ ID NO: 230) |
| [Switch D-E]-2-4F-22 | Ac-EKWKAFYDKVADKFFEAF-NH₂ |
| | (SEQ ID NO: 231) |
| [Switch D-E]-3-4F-22 | Ac-DKWKAFYEKVADKFFEAF-NH₂ |
| | (SEQ ID NO: 232) |
| [Switch D-E]-4-4F-22 | Ac-DKWKAFYEKVAEKFFDAF-NH₂ |
| | (SEQ ID NO: 233) |
| [K-2, W10, V-13] | |
| 4F-23 | Ac-DKFKAFYDKWAEVFKEAF-NH₂ |
| | (SEQ ID NO: 234) |
| [Switch D-E]-4F analogs | |
| [Switch D-E]-1-4F-23 | Ac-EKFKAFYEKWADVFKDAF-NH₂ |
| | (SEQ ID NO: 235) |
| [Switch D-E]-2-4F-23 | Ac-EKFKAFYDKWADVFKEAF-NH₂ |
| | (SEQ ID NO: 236) |
| [Switch D-E]-3-4F-23 | Ac-DKFKAFYEKWADVFKEAF-NH₂ |
| | (SEQ ID NO: 237) |
| [Switch D-E]-4-4F-23 | Ac-DKFKAFYEKWAEVFKDAF-NH₂ |
| | (SEQ ID NO: 238) |
| [K-2, F-13, W-14 4F] | |
| 4F-24 | Ac-DKFKAFYDKVAEFWKEAF-NH₂ |
| | (SEQ ID NO: 239) |
| [Switch D-E]-4F analogs | |
| [Switch D-E]-1-4F-24 | Ac-EKFKAFYEKVADFWKDAF-NH₂ |
| | (SEQ ID NO: 240) |
| [Switch D-E]-2-4F-24 | Ac-EKF1CAFYDKVADFWKEAF-NH₂ |
| | (SEQ ID NO: 241) |
| [Switch D-E]-3-4F-24 | Ac-DKFKAFYEKVADFWKEAF-NH₂ |
| | (SEQ ID NO: 242) |
| [Switch D-E]-4-4F-24 | Ac-DKFKAFYEKVAEFWKDAF-NH₂ |
| | (SEQ ID NO: 243) |
| Reverse 4F analogs | |
| Rev-4F | Ac-FAEKFKEAVKDYFAKFWD-NH₂ |
| | (SEQ ID NO: 244) |
| [Switch D-E]-1-Rev-4F | Ac-FADKFKDAVKEYFAKFWE-NH₂ |
| | (SEQ ID NO: 245) |
| [Switch D-E]-2-Rev-4F | Ac-FADKFKEAVKDYFAKFWE-NH₂ |
| | (SEQ ID NO: 246) |
| [Switch D-E]-3-Rev-4F | Ac-FAEKFKDAVKEYFAKFWD-NH₂ |
| | (SEQ ID NO: 247) |
| [Switch D-E]-4-Rev-4F | Ac-FAEKFKDAVKDYFAKFWE-NH₂ |
| | (SEQ ID NO: 248) |
| [A-2 and W-17 switched] | |
| Rev-4F-1 | Ac-FWEKFKEAVKDYFAKFAD-NH₂ |
| | (SEQ ID NO: 249) |
| [Switch D-E]-1-Rev-4F-1 | Ac-FWDKFKDAVKEYFAKFAE-NH₂ |
| | (SEQ ID NO: 250) |
| [Switch D-E]-2-Rev-4F-1 | Ac-FADKFKEAVKDYFAKFWE-NH₂ |
| | (SEQ ID NO: 251) |
| [Switch D-E]-3-Rev-4F-1 | Ac-FAEKFKDAVKEYFAKFWD-NH₂ |
| | (SEQ ID NO: 252) |
| [Switch D-E]-4-Rev-4F-1 | Ac-FAEKFKDAVKDYFAKFWE-NH₂ |
| | (SEQ ID NO: 253) |
| [Switch A-2 and F-16] | |
| Rev-4F-2 | Ac-FFEKFKEAVKDYFAKAWD-NH₂ |
| | (SEQ ID NO: 254) |
| [Switch D-E]-1-Rev-4F-2 | Ac-FFDKFKDAVKEYFAKAWE-NH₂ |
| | (SEQ ID NO: 255) |
| [Switch D-E]-2-Rev-4F-2 | Ac-FFDKFKEAVKDYFAKAWE-NH₂ |
| | (SEQ ID NO: 256) |
| [Switch D-E]-3-Rev-4F-2 | Ac-FFEKFKDAVKEYFAKAWD-NH₂ |
| | (SEQ ID NO: 257) |
| [Switch D-E]-4-Rev-4F-2 | Ac-FFEKFKDAVKDYFAKAWE-NH₂ |
| | (SEQ ID NO: 258) |
| [switch F-5 and A-8] | |
| Rev-4F-3 | Ac-FAEKAKEFVKDYFAKFWD-NH₂ |
| | (SEQ ID NO: 259) |
| [Switch D-E]-1-Rev-4F-3 | Ac-FADKAKDFVKEYFAKFWE-NH₂ |
| | (SEQ ID NO: 260) |
| [Switch D-E]-2-Rev-4F-3 | Ac-FADKAKEFVKDYFAKFWE-NH₂ |
| | (SEQ ID NO: 261) |
| [Switch D-E]-3-Rev-4F-3 | Ac-FAEKAKDFVKEYFAKFWD-NH₂ |
| | (SEQ ID NO: 262) |
| [Switch D-E]-4-Rev-4F-3 | Ac-FAEKAKDFVKDYFAKFWE-NH₂ |
| | (SEQ ID NO: 263) |
| [Switch A-8 and V9] | |
| Rev-4F-4 | Ac-FAEKFKEVAKDYFAKFWD-NH₂ |
| | (SEQ ID NO: 264) |
| [Switch D-E]-1-Rev-4F-4 | Ac-FADKFKDVAKEYFAKFWE-NH₂ |
| | (SEQ ID NO: 265) |
| [Switch D-E]-2-Rev-4F-4 | Ac-FADKFKEVAKDYFAKFWE-NH₂ |
| | (SEQ ID NO: 266) |
| [Switch D-E]-3-Rev-4F-4 | Ac-FAEKFKDVAKEYFAKFWD-NH₂ |
| | (SEQ ID NO: 267) |
| [Switch D-E]-4-Rev-4F-4 | Ac-FAEKFKDVAKDYFAKFWE-NH₂ |
| | (SEQ ID NO: 268) |
| [Switch V-9 to Y-12] | |
| Rev-4F-5 | Ac-FAEKFKEAYKDVFAKFWD-NH₂ |
| | (SEQ ID NO: 267) |
| [Switch D-E]-1-Rev-4F-5 | Ac-FADKFKDAYKEVFAKFWE-NH₂ |
| | (SEQ ID NO: 268) |
| [Switch D-E]-2-Rev-4F-5 | Ac-FADKFKEAYKDVFAKFWE-NH₂ |
| | (SEQ ID NO: 269) |
| [Switch D-E]-3-Rev-4F-5 | Ac-FAEKFKDAYKEVFAKFWD-NH₂ |
| | (SEQ ID NO: 270) |
| [Switch D-E]-4-Rev-4F-5 | Ac-FAEKFKDAYKDVFAKFWE-NH₂ |
| | (SEQ ID NO: 271) |
| [Switch Y-12 and F-13] | |
| Rev-4F-6 | Ac-FAEKFKEAVKDFYAKFWD-NH₂ |
| | (SEQ ID NO: 272) |
| [Switch D-E]-1-Rev-4F-6 | Ac-FADKFKDAVKEFYAKFWE-NH₂ |
| | (SEQ ID NO: 273) |
| [Switch D-E]-2-Rev-4F-6 | Ac-FADKFKEAVKDFYAKFWE-NH₂ |
| | (SEQ ID NO: 274) |
| [Switch D-E]-3-Rev-4F-6 | Ac-FAEKFKDAVKEFYAKFWD-NH₂ |
| | (SEQ ID NO: 275) |
| [Switch D-E]-4-Rev-4F-6 | Ac-FAEKFKDAVKDFYAKFWE-NH₂ |
| | (SEQ ID NO: 276) |
| [Switch K-6 and W-17] | |
| Rev-4F-7 | Ac-FAEKFWEAVKDYFAKFKD-NH₂ |
| | (SEQ ID NO: 277) |
| [Switch D-E]-1-Rev-4F-7 | Ac-FADKFWDAVKEYFAKFKE-NH₂ |
| | (SEQ ID NO: 278) |
| [Switch D-E]-2-Rev-4F-7 | Ac-FADKFWEAVKDYFAKFKE-NH₂ |
| | (SEQ ID NO: 279) |
| [Switch D-E]-3-Rev-4F-7 | Ac-FAEKFWDAVKEYFAKFKD-NH₂ |
| | (SEQ ID NO: 280) |
| [Switch D-E]-4-Rev-4F-7 | Ac-FAEKFWDAVKDYFAKFKE-NH₂ |
| | (SEQ ID NO: 281) |
| [Switch F-1 and A-2] | |
| Rev-4F-8 | Ac-A FEKFKEAVKDYFAKFWD-NH₂ |
| | (SEQ ID NO: 282) |
| [Switch D-E]-1-Rev-4F-8 | Ac-AFDKFKDAVKEYFAKFWE-NH₂ |
| | (SEQ ID NO: 283) |
| [Switch D-E]-2-Rev-4F-8 | Ac-AFDKFKEAVKDYFAKFWE-NH₂ |
| | (SEQ ID NO: 284) |
| [Switch D-E]-3-Rev-4F-8 | Ac-AFEKFKDAVKEYFAKFWD-NH₂ |
| | (SEQ ID NO: 285) |
| [Switch D-E]-4-Rev-4F-8 | Ac-AFEKFKDAVKDYFAKFWE-NH₂ |
| | (SEQ ID NO: 286) |
| [F-1 and V-9 are switched] | |
| Rev-F-9 | Ac-VAEKFKEAFKDYFAKFWD-NH₂ |
| | (SEQ ID NO: 287) |
| [Switch D-E]-1-Rev-4F-9 | Ac-VADKFKDAFKEYFAKFWE-NH₂ |
| | (SEQ ID NO: 288) |
| [Switch D-E]-2-Rev-4F-9 | Ac-VADKFKEAFKDYFAKFWE-NH₂ |
| | (SEQ ID NO: 289) |
| [Switch D-E]-3-Rev-4F-9 | Ac-VAEKFKDAFKEYFAKFWD-NH₂ |
| | (SEQ ID NO: 290) |
| [Switch D-E]-4-Rev-4F-9 | Ac-VAEKFKDAFKDYFAKFWE-NH₂ |
| | (SEQ ID NO: 291) |
| [F-1 and Y-12 are switched] | |
| Rev-4F-10 | Ac-YAEKFKEAVKDFFAKFWD-NH₂ |
| | (SEQ ID NO: 292) |
| [Switch D-E]-1-Rev-4F-10 | Ac-YADKFKDAVKEFFAKFWE-NH₂ |
| | (SEQ ID NO: 293) |
| [Switch D-E]-2-Rev-4F-10 | Ac-YADKFKEAVKDFFAKFWE-NH₂ |
| | (SEQ ID NO: 294) |
| [Switch D-E]-3-Rev-4F-10 | Ac-YAEKFKDAVKEFFAKFWD-NH₂ |
| | (SEQ ID NO: 295) |
| [Switch D-E]-4-Rev-4F-10 | Ac-YAEKFKDAVKDFFAKFWE-NH₂ |
| | (SEQ ID NO: 296) |
| [F-1 and A-8 are switched] | |
| Rev-4F-11 | Ac-AAEKFKEFVKDYFAKFWD-NH₂ |
| | (SEQ ID NO: 297) |
| [Switch D-E]-1-Rev-4F-11 | Ac-AADKFKDFVKEYFAKFWE-NH₂ |
| | (SEQ ID NO: 298) |
| [Switch D-E]-2-Rev-4F-11 | Ac-AADKFKEFVKDYFAKFWE-NH₂ |
| | (SEQ ID NO: 299) |
| [Switch D-E]-3-Rev-4F-11 | Ac-AAEKFKDFVKEYFAKFWD-NH₂ |
| | (SEQ ID NO: 300) |
| Switch D-E]-4-Rev-4F-11 | Ac-AAEKFKDFVKDYFAKFWE-NH₂ |
| | (SEQ ID NO: 301) |
| [A-2 and F-5 are switched] | |
| Rev-4F-12 | Ac-FFEKAKEAVKDYFAKFWD-NH₂ |
| | (SEQ ID NO: 302) |
| [Switch D-E]-1-Rev-4F-12 | Ac-FFDKAKDAVKEYFAKFWE-NH₂ |
| | (SEQ ID NO: 303) |
| [Switch D-E]-2-Rev-4F-12 | Ac-FFDKAKEAVKDYFAKFWE-NH₂ |
| | (SEQ ID NO: 304) |
| [Switch D-E]-3-Rev-4F-12 | Ac-141-EKAKDAVKEYFAKFWD- |
| | NH₂ (SEQ ID NO: 305) |
| [Switch D-E]-4-Rev-4F-12 | Ac-1-1-EKAKDAVKDYFAKFWE-NH₂ |
| | (SEQ ID NO: 306) |
| [A-2 and Y12 are switched | |
| Rev-4F-13 | Ac-FYEKFKEAVKDAFAKFWD-NH₂ |
| | (SEQ ID NO: 307) |
| [Switch D-E]-1-Rev-4F-13 | Ac-FYDKFKDAVKEAFAKFWE-NH₂ |
| | (SEQ ID NO: 308) |
| [Switch D-E]-2-Rev-4F-13 | Ac-FYDKFKEAVKDAFAKFWE-NH₂ |
| | (SEQ ID NO: 309) |
| [Switch D-E]-3-Rev-4F-13 | Ac-FYEKFKDAVKEAFAKFWD-NH₂ |
| | (SEQ ID NO: 310) |
| [Switch D-E]-4-Rev-4F-13 | Ac-FYEKFKDAVKDAFAKFWE-NH₂ |
| | (SEQ ID NO: 311) |
| [A-2 and V-9 are switched] | |
| Rev-4F-14 | Ac-FVEKFKEAAKDYFAKFWD-NH₂ |
| | (SEQ ID NO: 312) |
| [Switch D-E]-1-Rev-4F-14 | Ac-FVDKFKDAAKEYFAKFWE-NH₂ |
| | (SEQ ID NO: 313) |
| [Switch D-E]-2-Rev-4F-14 | Ac-FVDKFKEAAKDYFAKFWE-NH₂ |
| | (SEQ ID NO: 314) |
| [Switch D-E]-3-Rev-4F-14 | Ac-FVEKFKDAAKEYFAKFWD-NH₂ |
| | (SEQ ID NO: 315) |
| [Switch D-E]-4-Rev-4F-14 | Ac-FVEKFKDAAKDYFAKFWE-NH₂ |
| | (SEQ ID NO: 316) |
| [F-5 and Y-12 are switched] | |
| Rev-4F-15 | Ac-FAEKYKEAVKDFFAKFWD-NH₂ |
| | (SEQ ID NO: 317) |
| [Switch D-E]-1-Rev-4F-15 | Ac-FADKYKDAVKEFFAKFWE-NH₂ |
| | (SEQ ID NO: 318) |
| [Switch D-E]-2-Rev-4F-15 | Ac-FADKYKEAVKDFFAKFWE-NH₂ |
| | (SEQ ID NO: 319) |
| [Switch D-E]-3-Rev-4F-15 | Ac-FAEKYKDAVKEFFAKFWD-NH₂ |
| | (SEQ ID NO: 320) |
| [Switch D-E]-4-Rev-4F-15 | Ac-FAEKYKDAVKDFFAKFWE-NH₂ |
| | (SEQ ID NO: 321) |
| [F-5 and V-9 are switched] | |
| Rev-4F-16 | Ac-FAEKVKEAFKDYFAKFWD-NH₂ |
| | (SEQ ID NO: 322) |
| [Switch D-E]-1-Rev-4F-16 | Ac-FADKVKDAFKEYFAKFWE-NH₂ |
| | (SEQ ID NO: 323) |
| [Switch D-E]-2-Rev-4F-16 | Ac-FADKVKEAFKDYFAKFWE-NH₂ |
| | (SEQ ID NO: 324) |
| [Switch D-E]-3-Rev-4F-16 | Ac-FAEKVKDAFKEYFAKFWD-NH₂ |
| | (SEQ ID NO: 325) |
| [Switch D-E]-4-Rev-4F-16 | Ac-FAEKVKDAFKDYFAKFWE-NH₂ |
| | (SEQ ID NO: 326) |
| [A-8 and Y-12 switched] | |
| Rev-4F-17 | Ac-FAEKFKEYVKDAFAKFWD-NH₂ |
| | (SEQ ID NO: 327) |
| [Switch D-E]-1-Rev-4F-17 | Ac-FADKFKDYVKEAFAKFWE-NH₂ |
| | (SEQ ID NO: 328) |
| [Switch D-E]-2-Rev-4F-17 | Ac-FADKFKEYVKDAFAKFWE-NH₂ |
| | (SEQ ID NO: 329) |
| [Switch D-E]-3-Rev-4F-17 | Ac-FAEKFKDYVKEAFAKFWD-NH₂ |
| | (SEQ ID NO: 330) |
| [Switch D-E]-4-Rev-4F-17 | Ac-FAEKFKDYVKDAFAKFWE-NH₂ |
| | (SEQ ID NO: 331) |
| [V-9 and F-13 are switched] | |
| Rev-4F-18 | Ac-FAEKFKEAFKDYVAKFWD-NH₂ |
| | (SEQ ID NO: 332) |
| [Switch D-E]-1-Rev-4F-18 | Ac-FADKFKDAFKEYVAKFWE-NH₂ |
| | (SEQ ID NO: 333) |
| [Switch D-E]-2-Rev-4F-18 | Ac-FADKFKEAFKDYVAKFWE-NH₂ |
| | (SEQ ID NO: 334) |
| [Switch D-E]-3-Rev-4F-18 | Ac-FAEKFKDAFKEYVAKFWD-NH₂ |
| | (SEQ ID NO: 335) |
| [Switch D-E]-4-Rev-4F-18 | Ac-FAEKFKDAFKDYVAKFWE-NH₂ |
| | (SEQ ID NO: 336) |
| [V-9 and F-16 switched] | |
| Rev-4F-19 | Ac-FAEKFKEAFKDYFAKVWD-NH₂ |
| | (SEQ ID NO: 337) |
| [Switch D-E]-1-Rev-4F-19 | Ac-FADKFKDAFKEYFAKVWE-NH₂ |
| | (SEQ ID NO: 338) |
| [Switch D-E]-2-Rev-4F-19 | Ac-FADKFKEAFKDYFAKVWE-NH₂ |
| | (SEQ ID NO: 339) |
| [Switch D-E]-3-Rev-4F-19 | Ac-FAEKFKDAFKEYFAKVWD-NH₂ |
| | (SEQ ID NO: 340) |
| Switch D-E]-4-Rev-4F-19 | Ac-FAEKFKDAFKDYFAKVWE-NH₂ |
| | (SEQ ID NO: 341) |
| [Y-12 and F-16 are switched | |
| Rev-4F-20 | Ac-FAEKFKEAVKDFFAKYWD-NH₂ |
| | (SEQ ID NO: 342) |
| [Switch D-E]-1-Rev-4F-20 | Ac-FADKFKDAVKEFFAKYWE-NH₂ |
| | (SEQ ID NO: 343) |
| [Switch D-E]-2-Rev-4F-20 | Ac-FADKFKEAVKDFFAKYWE-NH₂ |
| | (SEQ ID NO: 344) |
| [Switch D-E]-3-Rev-4F-20 | Ac-FAEKFKDAVKEFFAKYWD-NH₂ |
| | (SEQ ID NO: 345) |
| [Switch D-E]-4-Rev-4F-20 | Ac-FAEKFKDAVKDFFAKYWE-NH₂ |
| | (SEQ ID NO: 346) |
| [W-1, F-6 and K-17 Rev 4F] | |
| Rev-4F-21 | Ac-WAEKFFEAVKDYFAKFKD-NH₂ |
| | (SEQ ID NO: 347) |
| [Switch D-E]-1-Rev-4F-7 | Ac-WADKFFDAVKEYFAKFKE-NH₂ |
| | (SEQ ID NO: 348) |
| [Switch D-E]-2-Rev-4F-7 | Ac-WADKFFEAVKDYFAKFKE-NH₂ |
| | (SEQ ID NO: 349) |
| [Switch D-E]-3-Rev-4F-7 | Ac-WAEKFFDAVKEYFAKFKD-NH₂ |
| | (SEQ ID NO: 350) |
| Switch D-E]-4-Rev-4F-7 | Ac-WAEKFFDAVKDYFAKFKE-NH₂ |
| | (SEQ ID NO: 351) |
| [W-5, F-6 and K-17 Rev-4F] | |
| Rev-4F-22 | Ac-FAEKWFEAVKDYFAKFKD-NH₂ |
| | (SEQ ID NO: 352) |
| [Switch D-E]-1-Rev-4F-22 | Ac-FADKWFDAVKEYFAKFKE-NH₂ |
| | (SEQ ID NO: 353) |
| [Switch D-E]-2-Rev-4F-22 | Ac-FADKWFEAVKDYFAKFKE-NH₂ |
| | (SEQ ID NO: 354) |
| [Switch D-E]-3-Rev-4F-22 | Ac-FAEKWFDAVKEYFAKFKD-NH₂ |
| | (SEQ ID NO: 355) |
| [Switch D-E]-4-Rev-4F-22 | Ac-FAEKWFDAVKDYFAKFKE-NH₂ |
| | (SEQ ID NO: 356) |
| [V-6, W-9, K-17 Rev-4F] | |
| Rev-4F-23 | Ac-FAEKFVEAWKDYFAKFKD-NH₂ |
| | (SEQ ID NO: 357) |
| [Switch D-E]-1-Rev-4F-23 | Ac-FADKFVDAWKEYFAKFKE-NH₂ |
| | (SEQ ID NO: 358) |
| [Switch D-E]-2-Rev-4F-23 | Ac-FADKFVEAWKDYFAKFKE-NH₂ |
| | (SEQ ID NO: 359) |
| [Switch D-E]-3-Rev-4F-23 | Ac-FAEKFVDAWKEYFAKFKD-NH₂ |
| | (SEQ ID NO: 360) |
| [Switch D-E]-4-Rev-4F-23 | Ac-FAEKFVDAWKDYFAKFKE-NH₂ |
| | (SEQ ID NO: 361) |
| [Y-2, A-4, W-12, K-17 Rev-4F] | |
| Rev-4F-24 | Ac-FYEKFAEAVKDWFAKFKD-NH₂ |
| | (SEQ ID NO: 362) |
| [Switch D-E]-1-Rev-4F-24 | Ac-FYDKFADAVKEWFAKFKE-NH₂ |
| | (SEQ ID NO: 363) |
| [Switch D-E]-2-Rev-4F-24 | Ac-FYDKFAEAVKDWFAKFKE-NH₂ |
| | (SEQ ID NO: 364) |
| [Switch D-E]-3-Rev-4F-24 | Ac-FYEKFADAVKEWFAKFKD-NH₂ |
| | (SEQ ID NO: 365) |
| [Switch D-E]-4-Rev-4F-24 | Ac-FYEKFADAVKDWFAKFKE-NH₂ |
| | (SEQ ID NO: 366) |

It is possible to readily identify biologically active and useful peptides. Thus, for example, the following peptides have been accurately identified as active: 3F1; 3F2; 4F the reverse (retro) forms thereof and the retro-inverso forms thereof. Lipid-associating peptides can comprise a peptide that is 18 amino acids in length and forms a class A amphipathic helix where the peptide has the amino acid composition 2 aspartates, 2 glutamates, 4 lysines, 1 tryptophan, 1 tyrosine, no more than one leucine, no more than 1 valine, no less than 1 and no more than 3 alanines, and with 3 to 6 amino acids from the group: phenylalanine, alpha-naphthalanine, beta-naphthalanine, histidine, and contains either 9 or 10 amino acids on the polar face in a helical wheel representation of the class A amphipathic helix including 4 amino acids with positive charge at neutral pH with two of the positively charged residues residing at the interface between the polar and non-polar faces and with two of the four positively charged residues on the polar face that are contiguous and on the non-polar face two of the amino acid residues from the group: phenylalanine, alpha-naphthalanine, beta-naphthalanine, histidine are also contiguous and if there are 4 or more amino acids from this group on the non-polar face there are also at least 2 residues from this group that are not contiguous.

Certain class Y as well as class A amphipathic helical peptides are disclosed. Class Y amphipathic helical peptides are known to those of skill in the art (see, e.g., Segrest et al. (1992) J. Lipid Res. 33: 141-166; Oram and Heinecke (2005) Physiol Rev. 85: 1343-1372, and the like). These peptides include, but are not limited to, an 18 amino acid peptide that forms a class A amphipathic helix or a class Y amphipathic helix described by formula I:

I

(SEQ ID NO: 367)

D X X K Y X X D K X Y D K X K D Y X

where the D's are independently Asp or Glu; the Ks are independently Lys or Arg; the Xs are independently Leu, norLeu, Val, Ile, Trp, Phe, Tyr, β-Nal, or α-Nal and all X residues are on the non-polar face (e.g., when viewed in a helical wheel diagram) except for one that can be on the polar face between two K residues; the Y's are independently Ala, His, Ser, Gln, Asn, or Thr non-polar face (e.g., when viewed in a helical wheel diagram) and the Y's are independently one Ala on the polar face, one His, one Ser, one Gln one Asn, or one Thr on the polar face (e.g., when viewed in a helical wheel diagram), where no more than two K are be contiguous (e.g., when viewed in a helical wheel diagram); and where no more than 3 D's are contiguous (e.g., when viewed in a helical wheel diagram) and the fourth D is be separated from the other D's by a Y. Illustrative peptides of this kind which include peptides with histidine, and/or alpha- and/or beta-napthalanine are shown in Table 5. Reverse (retro-), inverse, retro-inverso-, and circularly permuted forms of these peptides are also contemplated.

TABLE 5

| Short Name | Peptide Sequence |
|---|---|
| [A-5 >10 H]4F | Ac-DWFKHFYDKVAEKFKEAF-NH₂ |
| | (SEQ ID NO: 368) |
| [A-5 > H, D-E switched] 4F | Ac-EWFKHFYEKVADKFKDAF-NH₂ |
| | (SEQ ID NO: 369) |
| [A-5 > H, D-1 > E]4F | Ac-EWFKHFYDKVAEKFKEAF-NH₂ |
| | (SEQ ID NO: 370) |
| [A-5 > H, D-8 > E]4-F | Ac-DWFKHFYEKVAEKFKEAF-NH₂ |
| | (SEQ ID NO: 371) |
| [A-5 > H, E-12 > D]4F | Ac-DWFKHFYDKVADKFKEAF-NH₂ |
| | (SEQ ID NO: 372) |
| [A-5 > H, E-16 > D]4F | Ac-DWFKHFYDKVAEKFKDAF-NH₂ |
| | (SEQ ID NO: 373) |
| [F-3 > H, A-5 > F]-4F | Ac-DWHKFFYDKVAEKFKEAF-NH₂ |
| | (SEQ ID NO: 374) |
| [F-3 > H, A-5 > F, D-E switched]-4F | Ac-EWHKFFYEKVADKFKDAF-NH₂ |
| | (SEQ ID NO: 375) |
| [F-3 > H, A-5 > F, D-1 > E]-4F | Ac-EWHKFFYDKVAEKFKEAF-NH₂ |
| | (SEQ ID NO: 376) |
| [F-3 > H, A-5 > F, D-8 > E]-4F | Ac-DWHKFFYEKVAEKFKEAF-NH₂ |
| | (SEQ ID NO: 377) |
| [F-3 > H, A-5 > F, E-12 > D]-4F | Ac-DWHKFFYDKVADKFKEAF-NH₂ |
| | (SEQ ID NO: 378) |
| [F-3 > H, A-5 > F, E-16 > D]-4F | Ac-DWHKFFYDKVAEKFKDAF-NH₂ |
| | (SEQ ID NO: 379) |
| [A-5 > F, F-6 > H]4F | Ac-DWFKFHYDKVAEKFKEAF-NH₂ |
| | (SEQ ID NO: 380) |
| [A-5 > F, F-6 > H, D-E switched 4F | Ac-EWFKFHYEKVADKFKDAF-NH₂ |
| | (SEQ ID NO: 381) |
| [[A-5 > F, F-6 > H, D-1 > E]4F | Ac-EWFKFHYDKVAEKFKEAF-NH₂ |
| | (SEQ ID NO: 382) |
| [A-5 > F, F-6 > H, D-8 > E]4F | Ac-DWFKFHYEKVAEKFKEAF-NH₂ |
| | (SEQ ID NO: 383) |
| [A-5 > F, F-6 > H, E-12 > D]4F | Ac-DWFKFHYDKVADKFKEAF-NH₂ |
| | (SEQ ID NO: 384) |
| [A-5 > F, F-6 > H, E-16 > D]4F | Ac-DWFKFHYDKVAEKFKDAF-NH₂ |
| | (SEQ ID NO: 385) |
| [A-5 > V, V-10 > H]4F | Ac-DWFKVFYDKHAEKFKEAF-NH₂ |
| | (SEQ ID NO: 386) |
| [A-5 > V, V-10 > H, D-E switched]4F | Ac-EWFKVFYEKHADKFKDAF-NH₂ |
| | (SEQ ID NO: 387) |
| [A-5 > V, V-10 > H, D-1 > E]4F | Ac-EWFKVFYDKHAEKFKEAF-NH₂ |
| | (SEQ ID NO: 388) |
| [A-5 > V, V-10 > H, D-8 > E]4F | Ac-DWFKVFYEKHAEKFKEAF-NH₂ |
| | (SEQ ID NO: 389) |
| [A-5 > V, V-10 > H, E-12 > D]4F | Ac-DWFKVFYDKHADKFKEAF-NH₂ |
| | (SEQ ID NO: 390) |
| [A-5 > V, V-10 > H, E16 > D]4F | Ac-DWFKVFYDKHAEKFKDAF-NH₂ |
| | (SEQ ID NO: 391) |
| [[A-17 > H]4F | Ac-DWFKAFYDKVAEKFKEHF-NH₂( [symbol] |
| | (SEQ ID NO: 392) |
| [A-17 > H, D-E switched]4F | Ac-EWFKAFYEKVADKFKDHF-NH₂ |
| | (SEQ ID NO: 393) |
| [[A-17 > H, D-1 > E]4F | Ac-EWFKAFYDKVAEKFKEHF-NH₂ |
| | (SEQ ID NO: 394) |
| [[A-17 > H, D-8 > E]4F | Ac-DWFKAFYEKVAEKFKEHF-NH₂ |
| | (SEQ ID NO: 395) |
| [[A-17 > H, E-12 > D]4F | Ac-DWFKAFYDKVADKFKEHF-NH₂ |
| | (SEQ ID NO: 396) |
| [[A-17 > H, E16 > D]4F | Ac-DWFKAFYDKVAEKFKDHF-NH₂ |
| | (SEQ ID NO: 397) |
| [A-17 > F, F-18 > H]4F | Ac-DWFKAFYDKVAEKFKEFH-NH₂ |
| | (SEQ ID NO: 398) |
| [A-17 > F, F-18 > H, D-E switched]4F | Ac-EWFKAFYEKVADKFKDFH-NH₂ |
| | (SEQ ID NO: 399) |
| [A-17 > F, F-18 > H, D-1 > E]-4F | Ac-EWFKAFYDKVAEKFKEFH-NH₂ |
| | (SEQ ID NO: 400) |
| [A-17 > F, F-18 > H]4F | Ac-DWFKAFYDKVAEKFKEFH-NH₂ |
| | (SEQ ID NO: 401) |
| [A-17 > F, F-18 > H, D-8 > E]-4F | Ac-DWFKAFYEKVAEKFKEFH-NH₂ |
| | (SEQ ID NO: 402) |
| [A-17 > F, F-18 > H, E-12 > D]4F | Ac-DWFKAFYDKVAEKFKEFH-NH₂ |
| | (SEQ ID NO: 403) |
| [A-17 > F, F-18 > H], E-16 > D]-4F | Ac-DWFKAFYDKVAEKFKDFH-NH₂( |
| | (SEQ ID NO: 404) |
| Rev-4F | Ac-FAEKFKEAVKDYFAKFWD-NH₂ |
| | (SEQ ID NO: 405) |
| [A-2 > H]Rev4F | Ac-FHEKFKEAVKDYFAKFWD-NH₂ |
| | (SEQ ID NO: 406) |
| Rev-[A-2 > H, D > E]-4F | Ac-FHEKFKEAVKEYFAKFWE-NH₂ |
| | (SEQ ID NO: 407) |
| Rev-[A-2 > H, E > D]4F | Ac-FHDKFKDAVKDYFAKFWD-NH₂ |
| | (SEQ ID NO: 408) |
| [A-2 > H, D-E switched]Rev-4F | Ac-FHDKFKDAVKEYFAKFWE-NH₂ |
| | (SEQ ID NO: 409) |
| [A-2 > H, E-3 > D]Rev-4F | Ac-FHDKFKEAVKDYFAKFWD-NH₂ |
| | (SEQ ID NO: 410) |
| [A-2 > H, E-7 > D]Rev-4F | Ac-FHEKFKDAVKDYFAKFWD-NH₂ |
| | (SEQ ID NO: 411) |
| [A-2 > 2H, D-11 > E]Rev-4F | Ac-FHEKFKEAVKEYFAKFWD-NH₂ |
| | (SEQ ID NO: 412) |
| [A-2 > H, D-18 > E]Rev-4F | Ac-FHEKFKEAVKDYFAKFWE-NH₂ |
| | (SEQ ID NO: 413) |
| [F-1 > H, A-2 > F]Rev-4F | Ac-HFEKFKEAVKDYFAKFWD-NH₂ |
| | (SEQ ID NO: 414) |
| [F-1 > H, A-2 > F, D-E switched]Rev-4F | Ac-HFDKFKDAVKEYFAKFWE-NH₂ |
| | (SEQ ID NO: 415) |
| [F-1 > H, A-2 > F, D > E]Rev-4F | Ac-HFEKFKEAVKEYFAKFWE-NH₂ |
| | (SEQ ID NO: 416) |
| [F-1 > H, A-2 > F, E-3 > D]Rev-4F | Ac-HFDKFKEAVKDYFAKFWD-NH₂ |
| | (SEQ ID NO: 417) |
| [F-1 > H, A-2 > F, E-7 > D]Rev-4F | Ac-HFEKFKDAVKDYFAKFWD-NH₂ |
| | (SEQ ID NO: 418) |
| [F-1 > H, A-2 > F, D-11 > E]Rev-4F | Ac-HFEKFKEAVKEYFAKFWD-NH₂ |
| | (SEQ ID NO: 419) |
| [F-1 > H, A-2 > F, D-18 > E]Rev-4F | Ac-HFEKFKEAVKDYFAKFWE-NH₂ |
| | (SEQ ID NO: 420) |
| [A-2 > F, F-5 > H] Rev D-4F | Ac-FFEKHKEAVKDYFAKFWD-NH₂ |
| | (SEQ ID NO: 421) |
| [A-2 > F, F-5 > H, D-E switched]Rev D-4F | Ac-FFDKHKDAVKEYFAKFWE-NH₂ |
| | (SEQ ID NO: 422) |
| [A-2 > F, F-5 > H, D > E]Rev D-4F | Ac-FFEKHKEAVKEYFAKFWE-NH₂ |
| | (SEQ ID NO: 423) |
| [A-2 > F, F-5 > H, E > D]Rev D-4F [ | Ac-FFDKHKDAVKDYFAKFWD-NH₂ |
| | (SEQ ID NO: 424) |
| A-2 > F, F-5 > H, E-3 > D]Rev | Ac-FFDKHKEAVKDYFAKFWD-NH₂ |
| | (SEQ ID NO: 425) |
| D-4F [A-2 > F, F-5 > H, D-11 > E]Rev D-4F | Ac-FFEKHKEAVKEYFAKFWD-NH₂ |
| | (SEQ ID NO: 426) |
| [A-2 > F, F-5 > H, D-18 > E]Rev D-4F | Ac-FFEKHKEAVKDYFAKFWE-NH₂ |
| | (SEQ ID NO: 427) |
| [A-2 > V, V-9 > H]Rev D-4F | Ac-FVEKFKEAHKDYFAKFWD-NH₂ |
| | (SEQ ID NO: 428) |
| [A-2 > V, V-9 > H, D-E switched]Rev D-4 | Ac-FVDKFKDAHKEYFAKFWE-NH₂ |
| | (SEQ ID NO: 429) |
| [A-2 > V, V-9 > H, D > E]Rev D-4F | Ac-FVEKFKEAHKEYFAKFWE-NH₂ |
| | (SEQ ID NO: 430) |
| [A-2 > V, V-9 > H, E > D]Rev D-4F | Ac-FVDKFKDAHKDYFAKFWD-NH₂ |
| | (SEQ ID NO: 431) |
| [A-2 > V, V-9 > H, E-3 > D]Rev D-4F | Ac-FVDKFKEAHKDYFAKFWD-NH₂ |
| | (SEQ ID NO: 432) |
| [A-2 > V, V-9 > H, E-7 > D]Rev D-4F | Ac-FVEKFKDAHKDYFAKFWD-NH₂ |
| | (SEQ ID NO: 433) |
| [A-2 > V, V-9 > H, D-11 > E]Rev D-4F | Ac-FVEKFKEAHKEYFAKFWD-NH₂ |
| | (SEQ ID NO: 434) |
| [A-2 > V, V-9 > H, D-18 > E]Rev D-4F | Ac-FVEKFKEAHKDYFAKFWE-NH₂ |
| | (SEQ ID NO: 435) |
| [A-8 > H]Rev-4F | Ac-FAEKFKEHVKDYFAKFWD-NH₂ |
| | (SEQ ID NO: 436) |
| [A-8 > H, D-E switched]Rev-4F | Ac-FADKFKDHVKEYFAKFWE-NH₂ |
| | (SEQ ID NO: 437) |
| [A-8 > H, D > E]Rev-4F | Ac-FAEKFKEHVKEYFAKFWE-NH₂ |
| | (SEQ ID NO: 438) |
| [A-8 > H, E > D]Rev-4F | Ac-FADKFKDHVKDYFAKFWD-NH₂ |
| | (SEQ ID NO: 439) |
| [A-8 > H, E-3 > D]Rev-4F | Ac-FADKFKEHVKDYFAKFWD-NH₂( |
| | (SEQ ID NO: 440) |
| [A-8 > H, E-7 > D]Rev-4F | Ac-FAEKFKDHVKDYFAKFWD-NH₂ |
| | (SEQ ID NO: 441) |
| [A-8 > H, D-11 > E]Rev-4F | Ac-FAEKFKEHVKEYFAKFWD-NH₂ |
| | (SEQ ID NO: 442) |
| [A-8 > H, D-18 > E]Rev-4F | Ac-FAEKFKEHVKDYFAKFWE-NH₂ |
| | (SEQ ID NO: 443) |
| [A-8 > F, F-13 > H]Rev-4F | Ac-FAEKFKEFVKDYHAKFWD-NH₂ |
| | (SEQ ID NO: 444) |
| [A-8 > F, F-13 > H, D-E switched]Rev-4F | Ac-FADKFKDFVKEYHAKFWE-NH₂ |
| | (SEQ ID NO: 445) |
| [A-8 > F, F-13 > H, E-3 > D]Rev-4F | Ac-FADKFKEFVKDYHAKFWD-NH₂ |
| | (SEQ ID NO: 446) |
| [A-8 > F, F-13 > H, E-7 > D]Rev-4F | Ac-FAEKFKDFVKDYHAKFWD-NH₂ |
| | (SEQ ID NO: 447) |
| [A-8 > F, F-13 > H, E > D]Rev-4F | Ac-FADKFKDFVKDYHAKFWD-NH₂ |
| | (SEQ ID NO: 448) |
| [A-8 > F, F-13 > H, D > E]Rev-4F | Ac-FAEKFKEFVKEYHAKFWE-NH₂ |
| | (SEQ ID NO: 449) |
| [A-8 > F, F-13 > H, D-11 > E]Rev-4F | Ac-FAEKFKEFVKEYHAKFWD-NH₂ |
| | (SEQ ID NO: 450) |
| [A-8 > F, F-13 > H, D-18 > E]Rev-4F | Ac-FAEKFKEFVKDYHAKFWE-NH₂ |
| | (SEQ ID NO: 451) |
| [A-8 > F, F16 > H]Rev-4F | Ac-FAEKFKEFVKDYFAKHWD-NH₂ |
| | (SEQ ID NO: 452) |
| [A-8 > F, F16 > H, D-E switched]Rev-4F | Ac-FADKFKDFVKEYFAKHWE-NH₂ |
| | (SEQ ID NO: 453) |
| [A-8 > F, F16 > H, D > E]Rev-4F | Ac-FAEKFKEFVKEYFAKHWE-NH₂ |
| | (SEQ ID NO: 454) |
| [A-8 > F, F16 > H, E > D]Rev-4F | Ac-FADKFKDFVKDYFAKHWD-NH₂ |
| | (SEQ ID NO: 455) |
| [A-8 > F, F16 > H, E-3 > D]Rev-4F | Ac-FADKFKEFVKDYFAKHWD-NH₂ |
| | (SEQ ID NO: 456) |
| [A-8 > F, F16 > H, E-7 > D]Rev-4F | Ac-FAEKFKDFVKDYFAKHWD-NH₂ |
| | (SEQ ID NO: 457) |
| [A-8 > F, F16 > H, D-11 > E]Rev-4F | Ac-FAEKFKEFVKEYFAKHWD-NH₂ |
| | (SEQ ID NO: 458) |
| [A-8 > F, F16 > H, D-18 > E]Rev-4F | Ac-FAEKFKEFVKDYFAKHWE-NH₂ |
| | (SEQ ID NO: 459) |

[symbol]

indicates data missing or illegible when filed

Examples of class A 4F and Rev 4F analogs with beta-Nph. Similarly, alpha-Nph analogs can be designed. Similarly to the above analogs, His can be incorporated to Nph analogs. D>E analogs, E>D analogs and D-E switch analogs are additional possibilities similarly to the above described analogs.

4Nph

(SEQ ID NO: 460)

Ac-DWNphKANphYDKVAEKNphKEANph-NH2
[D-E switched] 4Nph

(SEQ ID NO: 461)

Ac-EWNphKANphYEKVADKNphKDANph-NH2
[D > E]4Nph

(SEQ ID NO: 462)

Ac-EWNphKANphYEKVAEKNphKEANph-NH2
[E > D]4Nph

(SEQ ID NO: 463)

Ac-DWNphKANphYDKVADKNphKDANph-NH2
[D-1 > E]4Nph

(SEQ ID NO: 464)

Ac-EWNphKANphYDKVAEKNphKEALph-NH2
[D-8 > E]4Nph

(SEQ ID NO: 465)

Ac-DWNphKANphYEKVAEKNphKEANph-NH2
[E-12 > D]4Nph

(SEQ ID NO: 466)

Ac-DWNphKANphYDKVADKNphKEANph-NH2
[E-16 > D]4Nph

(SEQ ID NO: 467)

Ac-DWNphKANphYDKVAEKNphKDANph-NH2

As described above for 4Nph, a minimum of 7 additional analogs for each of the analogs given below.

[F-3, 6, > Nph]4F

(SEQ ID NO: 468)

Ac-DWNphKANphYDKVAEKFKEAF-NH2
[F-14, 18 > Nph]4F

(SEQ ID NO: 469)

Ac-DWFKAFYDKVAEKNphKEANph-NH2
[[F-3 > Nph]4F

(SEQ ID NO: 470)

Ac-DWNphKAFYDKVAEKFKEAF-NH2
[F-6 > Nph]4F

(SEQ ID NO: 471)

Ac-DWFKANphYDKVAEKFKEAF-NH2
[F-14 > Nph]4F

(SEQ ID NO: 472)

Ac-DWFKAFYDKVAEKNphKEAF-NH2
[F-18 > Nph]4F

(SEQ ID NO: 473)

Ac-DWFKAFYDKVAEKFKEANph-NH2

For each of the analog described below, a minimum of 7 additional analogs are possible as described above by switching D-E, D>E and E>D and single D or E analogs.

Rev-4Nph

(SEQ ID NO: 474)

Ac-NphAEKNphKEAVKDYNphAKNphWD-NH2
[F-3, 6 > Nph]Rev

(SEQ ID NO: 475)

Ac-NphAEKNphKEAVKDYFAKFWD-NH2
4F [F-13, 16]Rev-4F

(SEQ ID NO: 476)

Ac-FAEKFKEAVKDYNphAKNphWD-NH2
[F-3 > Nph]Rev-4F

(SEQ ID NO: 477)

Ac-NphAEKFKEAVKDYFAKFWD-NH2
[F-6 > Nph]Rev-4F

(SEQ ID NO: 478)

Ac-FAEKNphKEAVKDYFAKFWD-NH2
[F-13 > Nph]Rev-4F

(SEQ ID NO: 479)

Ac-FAEKFKEAVKDYNphAKFWD-NH2
[F-16 > Nph]Rev-4F

(SEQ ID NO: 480)

Ac-FAEKEKEAVKDYFAKNphWD-NH2

For the analogs described below, additional analogs are possible by incorporating His or alpha-Nph and beta-Nph

Rev-[D > E]-4F

(SEQ ID NO: 481)

Ac-FAEKFKEAVKEYFAKFWE-NH2
Rev-[E > D]4F

(SEQ ID NO: 482)

Ac-FADKFKDAVKDYFAKFWD-NH2
Rev-R4-4F

(SEQ ID NO: 483)

Ac-FAERFREAVKDYFAKFWD-NH2
Rev-R6-4F

(SEQ ID NO: 484)

Ac-FAEKFREAVKDYFAKFWD-NH2
Rev-R10-4F

(SEQ ID NO: 485)

Ac-FAEKFKEAVRDYFAKFWD-NH2
Rev-R14-4F

(SEQ ID NO: 486)

Ac-FAEKFKEAVKDYFARFWD-NH2
Rev-[D > E]-4F

(SEQ ID NO: 481)

Ac-FAEKFKEAVKEYFAKFWE-NH2
Rev-[E > D]4F

(SEQ ID NO: 482)

Ac-FADKFKDAVKDYFAKFWD-NH2
Rev-R4-4F

(SEQ ID NO: 483)

Ac-FAERFREAVKDYFAKFWD-NH2
Rev-R6-4F

(SEQ ID NO: 484)

Ac-FAEKFREAVKDYFAKFWD-NH2
Rev-R10-4F

(SEQ ID NO: 485)

Ac-FAEKFKEAVRDYFAKFWD-NH2
Rev-R14-4F

(SEQ ID NO: 486)

Ac-FAEKFKEAVKDYFARFWD-NH2
Rev-[D > E]-4F

(SEQ ID NO: 481)

Ac-FAEKFKEAVKEYFAKFWE-NH2
Rev-[E > D]4F

(SEQ ID NO: 482)

Ac-FADKFKDAVKDYFAKFWD-NH2
Rev-R4-4F

(SEQ ID NO: 483)

Ac-FAERFREAVKDYFAKFWD-NH2
Rev-R6-4F

(SEQ ID NO: 484)

Ac-FAEKFREAVKDYFAKFWD-NH2
Rev-R10-4F

(SEQ ID NO: 485)

Ac-FAEKFKEAVRDYFAKFWD-NH2
Rev-R14-4F

(SEQ ID NO: 486)

Ac-FAEKFKEAVKDYFARFWD-NH2
Rev-R4-4F

(SEQ ID NO: 483)

Ac-FAERFREAVKDYFAKFWD-NH2
Rev-R6-4F

(SEQ ID NO: 487)

Ac-FAEKFREAVKDYEAKFWD-NH2
Rev-R10-4F

(SEQ ID NO: 485)

Ac-FAEKFKEAVRDYFAKFWD-NH2
Rev-R14-4F

(SEQ ID NO: 486)

Ac-FAEKFKEAVKDYFARFWD-NH2
Rev-[D > E]-4F

(SEQ ID NO: 481)

Ac-FAEKFKEAVKEYFAKFWE-NH2
Rev-[E > D]4F

(SEQ ID NO: 482)

Ac-FADKFKDAVKDYFAKFWD-NH2
Rev-R4-4F

(SEQ ID NO: 483)

Ac-FAERFREAVKDYFAKFWD-NH2
Rev-R6-4F

(SEQ ID NO: 484)

Ac-FAEKFREAVKDYFAKFWD-NH2
Rev-R10-4F

(SEQ ID NO: 485)

Ac-FAEKFKEAVRDYFAKFWD-NH2
Rev-R14-4F

(SEQ ID NO: 486)

Ac-FAEKFKEAVKDYFARFWD-NH2

For each of the analogs below, additional H and Nph analogs are possible using the examples described above. Each analog can yield 7 analogs with the changes described in the examples given above.

Rev3F-2

(SEQ ID NO: 488)

Ac-LFEKFAEAFKDYVAKWKD-NH2
RevR4-3F-2

(SEQ ID NO: 489)

Ac-LFERFAEAFKDYVAKWKD-NH2
RevR10-3F2

(SEQ ID NO: 490)

Ac-LFEKFAEAFRDYVAKWKD-NH2
RevR15-3F-2

(SEQ ID NO: 491)

Ac-LFEKFAEAFKDYVARWKD-NH2
RevR17-3F-2

(SEQ ID NO: 492)

Ac-LFEKFAEAFKDYVAKWRD-NH2
Rev[D > E]3F2

(SEQ ID NO: 493)

Ac-LFEKFAEAFKEYVAKWKE-NH2
Rev[E > D]3F-2

(SEQ ID NO: 494)

Ac-LFDKFADAFKDYVAKWKD-NH2
Rev-[E3 > D]-3F-2

(SEQ ID NO: 495)

Ac-LFDKFAEAFKDYVAKWKD-NH2
Rev-[E7 > D]-3F-2

(SEQ ID NO: 496)

Ac-LFEKFADAFKDYVAKWKD-NH2
Rev[D11 > E]3F-2

(SEQ ID NO: 497)

Ac-LFEKFAEAFKEYVAKWKD-NH2
Rev-[D18 > E]3F-2

(SEQ ID NO: 498)

Ac-LFEKFAEAFKDYVAKWKE-NH2
Rev3F-1

(SEQ ID NO: 499)

Ac-FAEKAWEFVKDYFAKLKD-NH2
RevR4-3F-1

(SEQ ID NO: 500)

Ac-FAERAWEFVKDYFAKLKD-NH2
RevR10-3F-1

(SEQ ID NO: 501)

Ac-FAEKAWEFVKDYFAKLKD-NH2
RevR15-3F-1

(SEQ ID NO: 502)

Ac-FAEKAWEFVKDYFAKLKD-NH2
RevR17-3F-1

(SEQ ID NO: 503)

Ac-FAEKAWEFVKDYFAKLRD-NH2
Rev[D > E]3F-1

(SEQ ID NO: 504)

Ac-FAEKAWEFVKEYFAKLKE-NH2
Rev[E > D}3F-1

(SEQ ID NO: 505)

Ac-FADKAWDFVKDYFAKLKD-NH2
Rev[E3 > D]-3F-1

(SEQ ID NO: 506)

Ac-FADKAWEFVKDYFAKLKD-NH2
Rev[E7 > D]3F-1

(SEQ ID NO: 507)

Ac-FAEKAWDFVKDYFAKLKD-NH2
Rev-[D11 > E]3F-1

(SEQ ID NO: 508)

Ac-FAEKAWEFVKEYFAKLKD-NH2
Rev-[D18 > E]3F-1

(SEQ ID NO: 509)

Ac-FAEKAWEFVKDYFAKLKE-NH2
Rev-5F

(SEQ ID NO: 510)

Ac-FFEKFKEFVKDYFAKLWD-NH2
Rev-[D > E]5F

(SEQ ID NO: 511)

Ac-FFEKFKEFVKEYFAKLWE-NH2
Rev-[E > D]5F

(SEQ ID NO: 512)

Ac-FFDKFKDFVKDYFAKLWD-NH2
Rev-R4-5F

(SEQ ID NO: 513)

Ac-FFERFKEFVKDYFAKLWD-NH2
Rev-R6-5F

(SEQ ID NO: 514)

Ac-FFEKFREFVKDYFAKLWD-NH2
Rev-R10-5F

(SEQ ID NO: 515)

Ac-FFEKFKEFVRDYFAKLWD-NH2
Rev-R15-5F

(SEQ ID NO: 516)

Ac-FFEKFKEFVKDYFARLWD-NH2
Rev-[E3 > D]-5F

(SEQ ID NO: 517)

Ac-FFDKFKEFVKDYFAKLWD-NH2
Rev-[E7 > D]5F

(SEQ ID NO: 518)

Ac-FFEKFKDFVKDYFAKLWD-NH2
Rev-[D11 > E]-5F

(SEQ ID NO: 519)

Ac-FFEKFKEFVKEYFAKLWD-NH2
Rev-[D18 > E]-5F

(SEQ ID NO: 520)

Ac-FFEKFKEFVKDYFAKLWE-NH2
Rev-5F-2

(SEQ ID NO: 521)

Ac-FLEKFKEFVKDYFAKFWD-NH2
Rev-[D > E]-5F-2

(SEQ ID NO: 522)

Ac-FLEKFKEFVKEYFAKFWE-NH2
Rev-[E > D]-5F-2

(SEQ ID NO: 523)

Ac-FLDKFKEFVKDYFAKFWD-NH2
Rev-[E3 > D]-5F-2

(SEQ ID NO: 524)

Ac-FLDKFKEFVKDYFAKFWD-NH2
Rev-[E7 > D]-5F-2

(SEQ ID NO: 525)

Ac-FLEKFKDFVKDYFAKFWD-NH2
Rev-[D11 > E]-5F-2

(SEQ ID NO: 526)

Ac-FLEKFKEFVKEYFAKFWD-NH2
Rev-[D18 > E]-5F-2

(SEQ ID NO: 527)

Ac-FLEKFKEFVKDYFAKFWE-NH2
Rev-R4-5F-2

(SEQ ID NO: 528)

Ac-FLERFKEFVKDYFAKFWD-NH2
Rev-R6-5F-2

(SEQ ID NO: 529)

Ac-FLEKFREFVKDYFAKFWD-NH2
RevR10-5F-2

(SEQ ID NO: 530)

Ac-FLEKFKEFVRDYFAKFWD-NH2
Rev-R16-5F-2

(SEQ ID NO: 531)

Ac-FLEKFKEFVKDYFARFWD-NH2
Rev-6F

(SEQ ID NO: 532)

Ac-FFEKFKEFFKDYFAKLWD-NH2
Rev-[D > E]-6F

(SEQ ID NO: 533)

Ac-FFEKFKEFFKEYFAKLWE-NH2
Rev-[E > D]-6F

(SEQ ID NO: 534)

Ac-FF [symbol] KFK [symbol] FFKDYFAKLWD-NH2
Rev-R4-6F

(SEQ ID NO: 535)

Ac-FFERFKEFFKDYFAKLWD-NH2
Rev-R6-6F

(SEQ ID NO: 536)

Ac-FFEKFREFFKDYFAKLWD-NH2
Rev-R10-6F

(SEQ ID NO: 537)

Ac-FFEKFKEFFRDYFAKLWD-NH2
Rev-R14-6F

(SEQ ID NO: 538)

Ac-FFERFKEFFKDYFARLWD-NH2
Rev-[E3 > D]-6F

(SEQ ID NO: 539)

Ac-FFDKFKEFFKDYFAKLWD-NH2
Rev-[E7 > D]-6F

(SEQ ID NO: 540)

Ac-FFEKEKDFFKDYFAKLWD-NH2
Rev-[D11 > E]-6F

(SEQ ID NO: 541)

Ac-FFEKFKEFFKEYFAKLWD-NH2
Rev-[D18 > E]-6F

(SEQ ID NO: 542)

Ac-FFEKFKEFFKDYFAKLWE-NH2
Rev-4F

(SEQ ID NO: 543)

Ac-FAEKFKEAVKDYFAKFWD-NH2
Rev-[D > E]-4F

(SEQ ID NO: 481)

Ac-FAEKFKEAVKEYFAKFWE-NH2
Rev-[E > D]4F

(SEQ ID NO: 482)

Ac-FADKFKDAVKDYFAKFWD-NH2
Rev-R4-4F

(SEQ ID NO: 483)

Ac-FAERFREAVKDYFAKFWD-NH2
Rev-R6-4F

(SEQ ID NO: 484)

Ac-FAEKFREAVKDYFAKFWD-NH2
Rev-R10-4F

(SEQ ID NO: 485)

Ac-FAEKFKEAVRDYFAKFWD-NH2
Rev-R14-4F

(SEQ ID NO: 486)

Ac-FAEKFKEAVKDYFARFWD-NH2
4F-2

(SEQ ID NO: 544)

Ac-DKWKAVYDKFAEAFKEFF-NH2
[D > E]-4F-2

(SEQ ID NO: 545)

Ac-EKWKAVYEKFAEAFKEFF-NH2
[E > D]-4F-2

(SEQ ID NO: 546)

Ac-DKWKAVYDKFADAFKDFF-NH2
R2-4F-2

(SEQ ID NO: 547)

Ac-DRWKAVYDKFAEAFKEFF-NH2
R4-4F-2

(SEQ ID NO: 548)

Ac-DKWRAVYDKFAEAFKEFF-NH2
R9-4F-2

(SEQ ID NO: 549)

Ac-DKWKAVYDRFAEAFKEFF-NH2
R14-4F-2

(SEQ ID NO: 550)

Ac-DKWKAVYDKFAEAFREFF-NH2
Rev4F-2

(SEQ ID NO: 551)

Ac-FFEKFAEAFKDYVAKWKD-NH2
Rev-[D > E]-4F-2

(SEQ ID NO: 552)

Ac-FFEKFAEAFKEYVAKWKE-NH2
Rev-[E > D]-3F-2

(SEQ ID NO: 553)

Ac-FFDKFADAFKDYVAKWKD-NH2
Rev-R4-4F-2

(SEQ ID NO: 554)

Ac-FFERFAEAFKDYVAKWKD-NH2
Rev-R10-4F-2

(SEQ ID NO: 555)

Ac-EFERFAEAFRDYVAKWKD-NH2
Rev-R15-4F-2

(SEQ ID NO: 556)

Ac-FFEKFAEAFKDYVARWKD-NH2
Rev-R17-4F-2

(SEQ ID NO: 557)

Ac-FFERFAEAFKDYVAKWRD-NH2
Rev-[E3 > D]-4F-2

(SEQ ID NO: 558)

Ac-FFDKFAEAFKDYVAKWKD-NH2
Rev-[E7 > D]-4F-2

(SEQ ID NO: 559)

Ac-FFEKFADAFKDYVAKWKD-NH2
Rev-[D11 > E]-4F-2

(SEQ ID NO: 560)

Ac-FFERFAEAFKEYVAKWKD-NH2
Rev-[D18 > E]-4F-2

(SEQ ID NO: 561)

Ac-FFERFAEAFKDYVAKWKE-NH2
Rev-7F

(SEQ ID NO: 562)

Ac-FFEKFKEFFKDYFAKFWD-NH2
Rev-[E > D]-7F

(SEQ ID NO: 563)

Ac-FFDKFKDFFKDYFAKFWD-NH2
Rev-[D > E]-7F

(SEQ ID NO: 564)

Ac-FFEKFKEFFKEYFAKFWE-NH2
Rev-R4-7F

(SEQ ID NO: 565)

Ac-FFERFKEFFKDYFAKFWD-NH2
Rev-R6-7F

(SEQ ID NO: 566)

Ac-FFEKFREFFKDYFAKFWD-NH2
Rev-R10-7F

(SEQ ID NO: 567)

Ac-FFEKFKEFFRDYFAKFWD-NH2
Rev-R14-7F

(SEQ ID NO: 568)

Ac-FFEKFKEFFKDYFARFWD-NH2
Rev-[E3 > D]-7F

(SEQ ID NO: 569)

Ac-FFDKFKEFFKDYFAKFWD-NH2
Rev-[E7 > D]7F

(SEQ ID NO: 570)

Ac-FFEKFKDFFKDYFAKFWD-NH2
Rev-[D11 > E]-7F

(SEQ ID NO: 571)

Ac-FFEKFKEFFKEYFAKFWD-NH2
Rev-[D18 > E]-7F

(SEQ ID NO: 572)

Ac-FFEKFKEFFKDYFAKFWE-NH2

It is also noted that any of the peptides described herein can comprise non-natural amino acids in addition to or instead of the corresponding the natural amino acids identified herein. Such modifications include, but are not limited to acetylation, amidation, formylation, methylation, sulfation, and the like. Illustrative non-natural amino acids include, but are not limited to Ornithine, norleucine, norvaline, N-methylvaline, 6-N-methyllysine, N-methylisoleucine, N-methylglycine, sarcosine, inosine, allo-isoleucine, isodesmolysine, 4-hydroxyproline, 3-hydroxyproline, allo-hydroxylysine, hydoxylisine, N-ethylasparagine, N-ethylglycine, 2,3-diaminopropionic acid, 2,2′-diaminopropionic acid, desmosine, 2,4-diaminobutyric acid, 2-aminopimelic acid, 3-aminoisobutyric acid, 2-aminoisobutyric acid, 2-aminoheptanoic acid, 6-aminocaproic acid, 4-aminobutyric acid, 2-aminobutyric acid, beta-alanine, 3-aminoadipic acid, 2-aminoadipic acid, and the like. In certain embodiments and one or more of the “natural” amino acids of the peptides described herein, can be substituted with the corresponding non-natural amino acid (e.g. as describe above).

In certain embodiments, this invention contemplates particularly the use of modified lysines. Such modifications include, but are not limited to, biotin modification of epsilon lysines and/or methylation of the epsilon lysines. Illustrative peptide comprising epsilon methylated lysines include, but are not limited to: Ac-D-W-F-K(eCH₃)₂-A-F-Y-D-K(eCH₃)₂-V-A-E-K(eCH₃)—₂-F-K(eCH₃)₂-E-A-F-NH(CH₃)₂ (SEQ ID NO:573) and: Ac-DWFK(eCH₃)₂AFYDK(eCH₃)₂VAEK(eCH₃)₂FK(eCH₃)₂EAF-NH(CH₃) (SEQ ID NO:574). Other modified amino acids include but are not limited to ornithine analogs and homoaminoalanine analogs (instead of (CH₂)₄—NH₂ for Lys it can be —(CH₂)₂—NH₂ for Haa and —(CH₂)₃—NH₂ for Orn] and the like. It is noted that these modifications are illustrative and not intended to be limiting. Illustrative 4F analogues that possess modified amino acids are shown in Table 6.

TABLE 6
Illustrative 4F analogs that comprise modified amino acids.

εN-Dimethyl-Lys derivative of 4F (εN-Dime)
Ac-D-W-F-K(εN-Dime)-A-F-Y-D-K(εN-Dime)-V-A-E-K(εN-Dime)-F-
K(εN-Dime)-E-A-F-NH₂ (SEQ ID NO: 575)
Ac-D-W-F-K-(εN-Dime)-A-F-Y-D-K(εN-Dime)-V-A-E-K(εN-Dime)-F-
K((εN-Dime)-E-A-F-NH-Me (SEQ ID NO: 576)
Ac-D-W-F-K-(EN-Dime)-A-F-Y-D-K(EN-Dime)-V-A-E-K(EN-Dime)-F-
K(EN-Dime)-E-A-F-N-(Me)₂ (SEQ ID NO: 577)
εN-Diethyl-Lys derivatives of 4F (εN-Diet)
Ac-D-W-F-K(εN-Diet)-A-F-Y-D-K(εN-Diet)-V-A-E-K(εN-Diet)-F-K(εN-
Diet)-E-A-F-NH₂ (SEQ ID NO: 578)
Ac-D-W-F-K(εN-Diet)-A-F-Y-D-K(εN-Diet)-V-A-E-K(εN-Diet)-F-K(εN-
Diet)-E-A-F-NH-Et (SEQ ID NO: 579)
Ac-D-W-F-K(εN-Diet)-A-F-Y-D-K(εN-Diet)-V-A-E-K(εN-Diet)-F-K(εN-
Diet)-E-A-F-NH-(Et)₂ (SEQ ID NO: 580)
εN-Monomethyl-Lys derivative of 4F (εN-Me)
Ac-D-W-F-K(εN-Me)-A-F-Y-D-K(εN-Me)-V-A-E-K(εN-Me)-F-K(εN-
Me)-E-A-F-NH₂ (SEQ ID NO: 581)
Ac-D-W-F-K(εN-Me)-A-F-Y-D-K(εN-Me)-V-A-E-K(εN-Me)-F-K(εN-
Me)-E-A-F-NH-Me (SEQ ID NO: 582)
Ac-D-W-F-K(εN-Me)-A-F-Y-D-K(εN-Me)-V-A-E-K(εN-Me)-F-K(εN-
Me)-E-A-F-N-(Me)₂ (SEQ ID NO: 583)
εN-ethylLys derivative of 4F (εN-Et)
Ac-D-W-F-K(εN-Et)-A-F-Y-D-K(εN-E0-V-A-E-K(εN-Et)-F-K(εN-
Et)-E-A-F-NH₂ (SEQ ID NO: 584)
Ac-D-W-F-K(εN-Et)-A-F-Y-D-K(εN-E0-V-A-E-K(εN-Et)-F-K(εN-
E0-E-A-F-NH-Et (SEQ ID NO: 585)
Ac-D-W-F-K(εN-Et)-A-F-Y-D-K(εN-Et)-V-A-E-K(εN-Et)-F-K(εN-
Et)-E-A-F-NH-(Et)₂ (SEQ ID NO: 586)
HomoLys analogs of 4F (hK) (—CH₂)₅—NH₂
Ac-D-W-F-hK-A-F-Y-D-hK-V-A-E-hK-F-hK-E-A-F-NH₂ (SEQ ID
NO: 587)
Ac-D-W-F-hK(εN-Dime)-A-F-Y-D-hK(εN-Dime)-V-A-E-hK(εN-Dime)-F-
hK(εN-Dime)-E-A-F-NH₂ (SEQ ID NO: 588)
Ac-D-W-F-hK(εN-Dime)-A-F-Y-D-hK(εN-Dime)-V-A-E-hK(εN-Dime)-F-
hK(εN-Dime)-E-A-F-N-(Me)₂ (SEQ ID NO: 589)
Ac-D-W-F-hK(εN-Dime)-A-F-Y-D-hK(εN-Dime)-V-A-E-hK(εN-
Dime)-F-hK(εN-Dime)-E-A-F-NH-Me (SEQ ID NO: 590)
Ac-D-W-F-hK(εN-Diet)-A-F-Y-D-hK(εN-Diet)-V-A-E-hK(εN-Diet)-F-
hK(εN-Diet)-E-A-F-NH-Et (SEQ ID NO: 591)
Ac-D-W-F-hK(εN-Me)-A-F-Y-D-hK(εN-Me)-V-A-E-hK(εN-Me)-F-
hK(εN-Me)-E-A-F-NH₂ (SEQ ID NO: 592)
Ac-D-W-F-hK(εN-Me)-A-F-Y-D-hK(εN-Me)-V-A-E-hK(εN-Me)-F-
hK(εN-Me)-E-A-F-NH-Me (SEQ ID NO: 593)
Ac-D-W-F-hK(εN-Me)-A-F-Y-D-hK(εN-Me)-V-A-E-hK(εN-Me)-F-
hK(εN-Me)-E-A-F-N-(Me)₂ (SEQ ID NO: 594)
Ac-D-W-F-hK(εN-Et)-A-F-Y-D-hK(εN-Et)-V-A-E-hK(εN-Et)-F-
hK(εN-Et)-E-A-F-NH₂ (SEQ ID NO: 595)
Ac-D-W-F-hK(εN-Et-A-F-Y-D-hK(εN-Et)-V-A-E-hK(εN-Et)-F-
hK(εN-Et)-E-A-F-NH-Et (SEQ ID NO: 596)
Ac-D-W-F-hK(εN-Et)-A-F-Y-D-hK(εN-Et)-V-A-E-hK(εN-Et)-F-
hK(εN-Et)-E-A-F-NH-(Et)₂ (SEQ ID NO: 597)
4F analogs in which K is replaced O (O = Ornithine, —(CH₂)₃—NH₂)
Ac-D-W-F-O-A-F-Y-D-O-V-A-E-O-F-O-E-A-F-NH₂ (SEQ ID NO: 598)
Ac-D-W-F-O(δN-Dime)-A-F--Y-D-O(δN-Dime)-V-A-E-O(δN-Dime)-F-O(δN-
Dime)-E-A-F-NH₂ (SEQ ID NO: 599)
Ac-D-W-F-O(δN-Dime)-A-F-Y-D-)(δN-Dime)-V-A-E-O(δN-Dime)-F-O(δN-
Dime)-E-A-F-N-(Me)₂ (SEQ ID NO: 600)
Ac-D-W-F-O(δN-Dime)-A-F-Y-D-O(δN-Dime)-V-A-E-O(δN-Dime)-F-O(δN-Dime)-
E-A-F-NH-Me (SEQ ID NO: 601)
Ac-D-W-F-O(δN-Diet)-A-F-Y-D-O(δN-Diet)-V-A-E-O(δN-Diet)-F-O(δN-Diet)-E-
A-F-NH-Et (SEQ ID NO: 602)
Ac-D-W-F-O(δN-Me)-A-F-Y-D-O(δN-Me)-V-A-E-O(δN-Me)-F-O(δN-
Me)-E-A-F-NH₂ (SEQ ID NO: 603)
Ac-D-W-F-O(δN-Me)-A-F-Y-D-O(δN-Me)-V-A-E-O(δN-Me)-F-O(δN-
Me)-E-A-F-NH-Me (SEQ ID NO: 604)
Ac-D-W-F-O(δN-Me)-A-F-Y-D-O(δN-Me)-V-A-E-O(δN-Me)-F-O(δN-
Me)-E-A-F-N-(Me)₂ (SEQ ID NO: 605)
Ac-D-W-F-O(δN-Et)-A-F-Y-D-O(δN-Et)-V-A-E-O(δN-Et)-F-O(δN-
Et)-E-A-F-NH₂ (SEQ ID NO: 606)
Ac-D-W-F-O(δN-Et)-A-F-Y-D-O(δN-Et)-V-A-E-O(δN-Et)-F-O(δN-
Et)-E-A-F-NH-Et (SEQ ID NO: 607)
Ac-D-W-F-O(δN-Et)-A-F-Y-D-O(δN-Et)-V-A-E-OdεN-Et)-F-O(δN-Et)-E-
A-F-NH-(Et)₂ (SEQ ID NO: 608)

iii. Dual Domain Peptides

Dual domain peptides are also disclosed. Dual domain peptides can be synthetic Apo E mimetics, consisting of a combination of the disclosed receptor binding domains of apolipoprotein E and the disclosed lipid-associating peptides, wherein said receptor binding domain is covalently linked to said lipid-associating peptide.

Also disclosed are synthetic Apo E mimetics, consisting of a combination of the disclosed receptor binding domains of apolipoprotein B and the disclosed lipid-associating peptides, wherein said receptor binding domain is covalently linked to said lipid-associating peptide. Non-limiting examples of the disclosed synthetic Apo E mimetics are provided in Table 7. The disclosed synthetic Apo E mimetics can also be N-terminally protected using acetyl and amino groups.

TABLE 7
Non-limiting Examples of the Disclosed
Synthetic Apo E mimetics

| Receptor Binding | |
|---|---|
| Domains of Apo E | Lipid-Associating Peptides |
| LRKLRKRLLR | DWLKAFYDKVAEKLKEAF |
| (SEQ ID NO: 4) | (SEQ ID NO: 5) |
| LRKLRKRLLR | DWLKAFYDKVAEKLKEAF |
| (SEQ ID NO: 4) | (SEQ ID NO: 5) |
| LRKLRKRLLR | DWLKAFYDKVAEKLKEAF |
| (SEQ ID NO: 4) | (SEQ ID NO: 5) |
| LRKMRKRLMR | DWLKAFYDKVAEKLKEAF |
| (SEQ ID NO: 7) | (SEQ ID NO: 5) |
| LRKMRKRLMR | DWLKAFYDKVAEKLKEAF |
| (SEQ ID NO: 7) | (SEQ ID NO: 5) |
| LRKL [symbol] KRLLR | DWLKAFYDKVAEKLKEAF |
| (SEQ ID NO: 8) | (SEQ ID NO: 5) |
| LRNVRKRLVR | DWLKAFYDKVAEKLKEAF |
| (SEQ ID NO: 9) | (SEQ ID NO: 5) |
| MRKLRKRVLR | DWLKAFYDKVAEKLKEAF |
| (SEQ ID NO: 10) | (SEQ ID NO: 5) |
| LRRLRRRLLR | DWLKAFYDKVAEKLKEAF |
| (SEQ ID NO: 11) | (SEQ ID NO: 5) |
| LRKLRKR [symbol] R | DWLKAFYDKVAEKLKEAF |
| (SEQ ID NO: 12) | (SEQ ID NO: 5) |
| LRKLRKRLLR | DWFKAFYDKVAEKFKEAF |
| (SEQ ID NO: 4) | (SEQ ID NO: 16) |
| LRKLRKRLLR | DWFKAFYDKVAEKFKEAF |
| (SEQ ID NO: 4) | (SEQ ID NO: 16) |
| LRKLRKRLLR | DWFKAFYDKVAEKFKEAF |
| (SEQ ID NO: 4) | (SEQ ID NO: 16) |
| LRKMRKRLMR | DWFKAFYDKVAEKFKEAF |
| (SEQ ID NO: 7) | (SEQ ID NO: 16) |
| LRKMRKRLMR | DWFKAFYDKVAEKFKEAF |
| (SEQ ID NO: 7) | (SEQ ID NO: 16) |
| LRKL [symbol] KRLLR | DWFKAFYDKVAEKFKEAF |
| (SEQ ID NO: 8) | (SEQ ID NO: 16) |
| LRNVRKRLVR | DWFKAFYDKVAEKFKEAF |
| (SEQ ID NO: 9) | (SEQ ID NO: 16) |
| MRKLRKRVLR | DWFKAFYDKVAEKFKEAF |
| (SEQ ID NO: 10) | (SEQ ID NO: 16) |
| LRRLRRRLLR | DWFKAFYDKVAEKFKEAF |
| (SEQ ID NO: 11) | (SEQ ID NO: 16) |
| LRKLRKR [symbol] R | DWFKAFYDKVAEKFKEAF |
| (SEQ ID NO: 12) | (SEQ ID NO: 16) |

In one aspect of the invention, the synthetic Apo E mimetics, consist of: a receptor binding domain of apolipoprotein E comprising the amino acid sequence of LRKLRKRFFR (SEQ ID NO:12); and a lipid-associating peptide, wherein said receptor binding domain is covalently linked to said lipid-associating peptide. As such, the receptor binding domain replaced the two leucine (L) residues at positions 148 and 149 of LRKLRKRLLR (SEQ ID NO:4) (hApoE[141-150], LRKLRKRLLR; SEQ ID NO:4) with two phenylalanine (F) residues.

In another aspect of the invention, the synthetic Apo E mimetics, comprise: a lipid binding domain of apolipoprotein E comprising the amino acid sequence of E-W-L-K-A-F-Y-D-K-V-F-E-K-F-K-E-A-F (SEQ ID NO:28); and a receptor binding domain peptide, wherein said lipid binding domain is covalently linked to said receptor binding domain peptide. As such, the lipid binding domain replaced the two leucine (L) residues of DWLKAFYDKVAEKLKEAF (SEQ ID NO:5) (18A) with two phenylalanine (F) residues resulting in the sequence DWFKAFYDKVAEKFKEAF (SEQ ID NO:16), also referred to as modified 18A or m18A).

a. Domain Switched Peptides

Also disclosed are synthetic Apo E mimetics, consisting of a combination of the disclosed receptor binding domains of apolipoprotein E and the disclosed lipid-associating peptides, wherein said receptor binding domain is covalently linked to said lipid-associating peptide in a domain switched orientation. Also disclosed are synthetic Apo E mimetics, consisting of a combination of the disclosed receptor binding domains of apolipoprotein B and the disclosed lipid-associating peptides, wherein said receptor binding domain is covalently linked to said lipid-associating peptide in a domain switched orientation. These peptides can be referred to as “domain switched” “switched domain”, or “switched” peptides. For example, disclosed are synthetic Apo E mimetics, consisting of a combination of the disclosed receptor binding domains of apolipoprotein E and the disclosed lipid-associating peptides, wherein said receptor binding domain is covalently linked to said lipid-associating peptide in a domain switched orientation to those described above and in Table 7. Specifically, the lipid-associating peptide is covalently linked to the receptor binding domain of apolipoprotein E such that the lipid-associating peptide is at the N-terminus of the synthetic apolipoprotein E-mimicking peptide. Non-limiting examples of the disclosed synthetic Apo E mimetics are provided in Table 8.

TABLE 8
Non-limiting Examples of Disclosed Synthetic
Apo E mimetics

| | Receptor Binding |
|---|---|
| Lipid-Associating Peptides | Domains of Apo E |
| DWLKAFYDKVAEKLKEAF | LRKLRKRLLR (SEQ ID |
| (SEQ ID NO: 5) | NO: 6) |
| DWLKAFYDKVAEKLKEAF | LRKLRKRLLR (SEQ |
| (SEQ ID NO: 5) | ID NO: 6) |
| DWLKAFYDKVAEKLKEAF | LRKLRKRLLR (SEQ |
| (SEQ ID NO: 5) | ID NO: 6) |
| DWLKAFYDKVAEKLKEAF | LRKMRKRLMR (SEQ |
| (SEQ ID NO: 5) | ID NO: 7) |
| DWLKAFYDKVAEKLKEAF | LRKMRKRLMR (SEQ |
| (SEQ ID NO: 5) | ID NO: 7) |
| DWLKAFYDKVAEKLKEAF | LRKL [symbol] KRLLR (SEQ ID |
| (SEQ ID NO: 5) | NO: 8) |
| DWLKAFYDKVAEKLKEAF | LRNVRKRLVR (SEQ ID |
| (SEQ ID NO: 5) | NO: 9) |
| DWLKAFYDKVAEKLKEAF | MRKLRKRVLR (SEQ ID |
| (SEQ ID NO: 5) | NO: 10) |
| DWLKAFYDKVAEKLKEAF | LRRLRRRLLR (SEQ ID |
| (SEQ ID NO: 5) | NO: 11) |
| DWLKAFYDKVAEKLKEAF | LRKLRKR [symbol] R (SEQ ID |
| (SEQ ID NO: 5) | NO: 12) |
| DWFKAFYDKVAEKFKEAF | LRKLRKRLLR (SEQ ID |
| (SEQ ID NO: 16) | NO: 4) |
| DWFKAFYDKVAEKFKEAF | LRKLRKRLLR (SEQ ID |
| (SEQ ID NO: 16) | NO: 4) |
| DWFKAFYDKVAEKFKEAF | LRKLRKRLLR (SEQ ID |
| (SEQ ID NO: 16) | NO: 4) |
| DWFKAFYDKVAEKFKEAF | LRKMRKRLMR (SEQ |
| (SEQ ID NO: 16) | ID NO: 7) |
| DWFKAFYDKVAEKFKEAF | LRKMRKRLMR (SEQ |
| (SEQ ID NO: 16) | ID NO: 7) |
| DWFKAFYDKVAEKFKEAF | LRKL [symbol] KRLLR (SEQ ID |
| (SEQ ID NO: 16) | NO: 8) |
| DWFKAFYDKVAEKFKEAF | LRNVRKRLVR (SEQ ID |
| (SEQ ID NO: 16) | NO: 9) |
| DWFKAFYDKVAEKFKEAF | MRKLRKRVLR (SEQ ID |
| (SEQ ID NO: 16) | NO: 10) |
| DWFKAFYDKVAEKFKEAF | LRRLRRRLLR (SEQ ID |
| (SEQ ID NO: 16) | NO: 11) |
| DWFKAFYDKVAEKFKEAF | LRKLRKR [symbol] R (SEQ ID |
| (SEQ ID NO: 16) | NO: 12) |

The disclosed domain switched synthetic Apo E mimetics can also be N-terminally protected using acetyl and amino groups.

b. Peptides with Reverse Orientation

Also disclosed are synthetic Apo E mimetics, consisting of a combination of the disclosed receptor binding domains of apolipoprotein E and the disclosed lipid-associating peptides, wherein said receptor binding domain is covalently linked to said lipid-associating peptide in a reversed orientation. For example, disclosed are synthetic Apo E mimetics, consisting of a combination of the disclosed receptor binding domains of apolipoprotein E and the disclosed lipid-associating peptides, wherein either the sequence of the receptor binding domain or the sequence of the lipid-associating peptide or both sequences are in the reversed orientation. Also disclosed are synthetic Apo E mimetics, consisting of a combination of the disclosed receptor binding domains of apolipoprotein B and the disclosed lipid-associating peptides, wherein said receptor binding domain is covalently linked to said lipid-associating peptide in a reversed orientation. Non-limiting examples of the disclosed synthetic Apo E mimetics are provided in Table 9.

TABLE 9
Non-limiting Examples of Synthetic
Apo E mimetics

| Receptor Binding | |
|---|---|
| Domains of Apo E | Lipid-Associating Peptides |
| RLLRKRLKRL | DWLKAFYDKVAEKLKEAF |
| (SEQ ID NO: 609) | (SEQ ID NO: 5) |
| RLLRKRLKRL | DWLKAFYDKVAEKLKEAF |
| (SEQ ID NO: 609) | (SEQ ID NO: 5) |
| RLLRKRLKRL | DWLKAFYDKVAEKLKEAF |
| (SEQ ID NO: 609) | (SEQ ID NO: 5) |
| RMLRKRMKRL | DWLKAFYDKVAEKLKEAF |
| (SEQ ID NO: 610) | (SEQ ID NO: 5) |
| RMLRKRMKRL | DWLKAFYDKVAEKLKEAF |
| (SEQ ID NO: 610) | (SEQ ID NO: 5) |
| RLLRKPLKRL | DWLKAFYDKVAEKLKEAF |
| (SEQ ID NO: 611) | (SEQ ID NO: 5) |
| RVLRKRVNRL | DWLKAFYDKVAEKLKEAF |
| (SEQ ID NO: 612) | (SEQ ID NO: 5) |
| RLVRKRLKRM | DWLKAFYDKVAEKLKEAF |
| (SEQ ID NO: 613) | (SEQ ID NO: 5) |
| RLLRRRLRRL | DWLKAFYDKVAEKLKEAF |
| (SEQ ID NO: 614) | (SEQ ID NO: 5) |
| RFFRKRLKRL | DWLKAFYDKVAEKLKEAF |
| (SEQ ID NO: 615) | (SEQ ID NO: 5) |
| RLLRKRLKRL | DWFKAFYDKVAEKFKEAF |
| (SEQ ID NO: 609) | (SEQ ID NO: 16) |
| RLLRKRLKRL | DWFKAFYDKVAEKFKEAF |
| (SEQ ID NO: 609) | (SEQ ID NO: 16) |
| RLLRKRLKRL | DWFKAFYDKVAEKFKEAF |
| (SEQ ID NO: 609) | (SEQ ID NO: 16) |
| RMLRKRMKRL | DWFKAFYDKVAEKFKEAF |
| (SEQ ID NO: 610) | (SEQ ID NO: 16) |
| RMLRKRMKRL | DWFKAFYDKVAEKFKEAF |
| (SEQ ID NO: 610) | (SEQ ID NO: 16) |
| RLLRKPLKRL | DWFKAFYDKVAEKFKEAF |
| (SEQ ID NO: 611) | (SEQ ID NO: 16) |
| RVLRKRVNRL | DWFKAFYDKVAEKFKEAF |
| (SEQ ID NO: 612) | (SEQ ID NO: 16) |
| RLVRKRLKRM | DWFKAFYDKVAEKFKEAF |
| (SEQ ID NO: 613) | (SEQ ID NO: 16) |
| RLLRRRLRRL | DWFKAFYDKVAEKFKEAF |
| (SEQ ID NO: 614) | (SEQ ID NO: 16) |
| RFFRKRLKRL | DWFKAFYDKVAEKFKEAF |
| (SEQ ID NO: 615) | (SEQ ID NO: 16) |
| LRKLRKRLLR | FAEKLKEAVKDYFAKLWD |
| (SEQ ID NO: 4) | (SEQ ID NO: 616) |
| LRKLRKRLLR | FAEKLKEAVKDYFAKLWD |
| (SEQ ID NO: 4) | (SEQ ID NO: 616) |
| LRKLRKRLLR | FAEKLKEAVKDYFAKLWD |
| (SEQ ID NO: 4) | (SEQ ID NO: 616) |
| LRKMRKRLMR | FAEKLKEAVKDYFAKLWD |
| (SEQ ID NO: 7) | (SEQ ID NO: 616) |
| LRKMRKRLMR | FAEKLKEAVKDYFAKLWD |
| (SEQ ID NO: 7) | (SEQ ID NO: 616) |
| LRKLPKRLLR | FAEKLKEAVKDYFAKLWD |
| (SEQ ID NO: 4) | (SEQ ID NO: 616) |
| LRNVRKRLVR | FAEKLKEAVKDYFAKLWD |
| (SEQ ID NO: 9) | (SEQ ID NO: 616) |
| MRKLRKRVLR | FAEKLKEAVKDYFAKLWD |
| (SEQ ID NO: 10) | (SEQ ID NO: 616) |
| LRRLRRRLLR | FAEKLKEAVKDYFAKLWD |
| (SEQ ID NO: 6) | (SEQ ID NO: 616) |
| LRKLRKRFFR | FAEKLKEAVKDYFAKLWD |
| (SEQ ID NO: 12) | (SEQ ID NO: 616) |
| LRKLRKRLLR | FAEKFKEAVKDYFAKFWD |
| (SEQ ID NO: 4) | (SEQ ID NO: 617) |
| LRKLRKRLLR | FAEKFKEAVKDYFAKFWD |
| (SEQ ID NO: 4) | (SEQ ID NO: 617) |
| LRKLRKRLLR | FAEKFKEAVKDYFAKFWD |
| (SEQ ID NO: 4) | (SEQ ID NO: 617) |
| LRKMRKRLMR | FAEKFKEAVKDYFAKFWD |
| (SEQ ID NO: 7) | (SEQ ID NO: 617) |
| LRKMRKRLMR | FAEKFKEAVKDYFAKFWD |
| (SEQ ID NO: 7) | (SEQ ID NO: 617) |
| LRKLPKRLLR | FAEKFKEAVKDYFAKFWD |
| (SEQ ID NO: 8) | (SEQ ID NO: 617) |
| LRNVRKRLVR | FAEKFKEAVKDYFAKFWD |
| (SEQ ID NO: 9) | (SEQ ID NO: 617) |
| MRKLRKRVLR | FAEKFKEAVKDYFAKFWD |
| (SEQ ID NO: 10) | (SEQ ID NO: 617) |
| LRRLRRRLLR | FAEKFKEAVKDYFAKFWD |
| (SEQ ID NO: 4) | (SEQ ID NO: 617) |
| LRKLRKRFFR | FAEKFKEAVKDYFAKFWD |
| (SEQ ID NO: 12) | (SEQ ID NO: 617) |

c. Scrambled Peptides

Also disclosed are synthetic Apo E mimetics, consisting of: a receptor binding domain of apolipoprotein E and a lipid-associating peptide, wherein said receptor binding domain is covalently linked to said lipid-associating peptide, wherein the receptor binding domain of apolipoprotein E is scrambled. For example, disclosed is a synthetic apolipoprotein E-mimicking peptide, consisting of: a receptor binding domain of apolipoprotein E comprising the amino acid sequence of D-W-L-K-A-F-V-Y-D-K-V-F-K-L-K-E-F-F (SEQ ID NO:69); and a lipid-associating peptide, wherein said receptor binding domain is covalently linked to said lipid-associating peptide. Also disclosed are synthetic Apo E mimetics, consisting of: a receptor binding domain of apolipoprotein B and a lipid-associating peptide, wherein said receptor binding domain is covalently linked to said lipid-associating peptide, wherein the receptor binding domain of apolipoprotein B is scrambled.

Also disclosed are synthetic Apo E mimetics, consisting of: a receptor binding domain of apolipoprotein E and a lipid-associating peptide, wherein said receptor binding domain is covalently linked to said lipid-associating peptide, wherein the lipid-associating peptide is scrambled. For example, disclosed herein is a synthetic Apo E mimetics, comprising: a lipid binding domain of apolipoprotein E comprising the amino acid sequence of E-W-L-K-A-F-V-Y-E-K-V-F-K-L-K-E-F-F (SEQ ID NO:70) and a receptor binding domain peptide, wherein said lipid binding domain is covalently linked to said receptor binding domain peptide.

Also disclosed are synthetic Apo E mimetics, consisting of: a receptor binding domain of apolipoprotein E and a lipid-associating peptide of apolipoprotein E, wherein receptor binding domain is covalently linked to said lipid-associating peptide, wherein both the receptor binding domain and the lipid-associating peptide are scrambled. Non-limiting examples of the disclosed scrambled synthetic Apo E mimetics are provided in Table 10.

TABLE 10
Scrambled Synthetic Apolipoprotein E-Mimicking
Peptides

| | Receptor | |
|---|---|---|
| | Binding Domains | Lipid-Associating |
| Name | of Apo E | Peptides |
| hE-Sc18A | LRKLRKRLLR | KAFEEVLAKKFYDKALWD |
| (hE with | (SEQ ID NO: 4) | (SEQ ID NO: 618) |
| Sc18A also | | |
| referred to as | | |
| Sc2F) | | |
| SchE-18A | LRLLRKLKRR | DWLKAFYDKVAEKLKEAF |
| | (SEQ ID NO: 619) | (SEQ ID NO: 5) |

The disclosed scrambled synthetic Apo E mimetics can also be N-terminally and C-terminally protected using acetyl and amide groups. The disclosed scrambled synthetic Apo E mimetics can also be reverse-oriented as described above.

d. Linkages

Any suitable linker can be used in accordance with the present invention. The peptide linkages can be selected from the group consisting of: —CH₂NH—, —CH₂S—, —CH₂—CH₂—, —CH═CH— (cis and trans), —COCH₂—, —CH(OH)CH₂—, —CH₂SO—, etc. by methods known in the art and further described in the following references: Spatola (1983) p. 267 in Chemistry and Biochemistry of Amino Acids, Peptides, and Proteins, B. Weinstein, eds., Marcel Dekker, New York; Spatola (1983) Vega Data 1(3) Peptide Backbone Modifications. (general review); Morley (1980) Trends Pharm Sci pp. 463-468 (general review); Hudson et al. (1979) Int J PeptProt Res 14:177-185 (—CH₂NH—, CH₂CH₂—); Spatola et al. (1986) Life Sci 38:1243-1249 (—CH₂—S); Hann, (1982) J ChemSoc Perkin Trans I 307-314 (—CH—CH—, cis and trans); Almquist et al. (1980) J Med. Chem. 23:1392-1398 (—COCH₂—); Jennings-White et al. (1982) Tetrahedron Lett. 23:2533 (—COCH₂—); Szelke et al., European Appln. EP 45665 (1982) CA: 97:39405 (1982) (—CH(OH)CH2-); Holladay et al. (1983) Tetrahedron Lett 24:4401-4404 (—C(OH)CH₂—); and Hruby (1982) Life Sci., 31:189-199 (—CH₂—S—)).

One particularly preferred non-peptide linkage is —CH₂NH—. Such peptide mimetics may have significant advantages over polypeptide embodiments, including, for example: more economical production, greater chemical stability, enhanced pharmacological properties (half-life, absorption, potency, efficacy, etc.), reduced antigenicity, and others.

In one aspect, the linker is a cleavable linker. To give but a few examples, cleavable linkers include protease cleavable peptide linkers, nuclease sensitive nucleic acid linkers, lipase sensitive lipid linkers, glycosidase sensitive carbohydrate linkers, pH sensitive linkers, hypoxia sensitive linkers, photo-cleavable linkers, heat-labile linkers, enzyme cleavable linkers (e.g., esterase cleavable linker), ultrasound-sensitive linkers, x-ray cleavable linkers, etc.

iv. Variants

The receptor binding domain or the lipid-associating peptide can be modified or altered as described above. For example, the receptor binding domain or the lipid-associating peptide can be mutated, scrambled, and/or reverse-oriented. Any other modifications or alterations disclosed herein for the dual-domain polypeptides can also be used for the single-domain peptides.

Numerous other variants or derivatives of the peptides disclosed herein are also contemplated. For example, scrambled peptides can also be reverse-oriented, or can be in a switched orientation. Additionally, reverse-oriented peptides can be in a switched orientation. All other combinations of the disclosed peptides are also contemplated. Non-limiting examples of the peptides have been described herein (see Tables 1-5, for example). As used herein, the term “analog” is used interchangeably with “variant” and “derivative.” Variants and derivatives are well understood to those of skill in the art and can involve amino acid sequence modifications. Such, amino acid sequence modifications typically fall into one or more of three classes: substantial; insertional; or deletional variants. Insertions include amino and/or carboxyl terminal fusions as well as intrasequence insertions of single or multiple amino acid residues. Insertions ordinarily are smaller insertions than those of amino or carboxyl terminal fusions, for example, on the order of one to four residues. These variants ordinarily are prepared by site-specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once. Substitutions, deletions, insertions or any combination thereof may be combined to arrive at a final derivative or analog. Substitutional variants are those in which at least one residue has been removed and a different residue inserted in its place. Such substitutions generally are made in accordance with Tables 11 and 12 and are referred to as conservative substitutions.

Substantial changes in function or immunological identity are made by selecting substitutions that are less conservative than those in Table 11, i.e., selecting residues that differ more significantly in their effect on maintaining (a) the structure of the polypeptide backbone in the area of the substitution, for example as a sheet or helical conformation, (b) the charge or hydrophobicity of the molecule at the target site, or (c) the bulk of the side chain. The substitutions which in general are expected to produce the greatest changes in the protein properties are those in which: (a) the hydrophilic residue, e.g. seryl or threonyl, is substituted for (or by) a hydrophobic residue, e.g., leucyl, isoleucyl, phenylalanyl, valyl or alanyl; Tryptophan, Tyrosinyl (b) a cysteine or proline is substituted for (or by) any other residue; (c) a residue having an electropositive side chain, e.g., lysyl, arginyl, or hystidyl, is substituted for (or by) an electronegative residue, e.g. glutamyl or aspartyl; or (d) a residue having a bulky side chain, e.g., phenylalanine, is substituted for (or by) one not having a side chain, e.g., glycine, in this case, or (e) by increasing the number of sites for sulfation and/or glycosylation.

TABLE 11
Amino Acid Substitutions

| | Non-limiting Exemplary |
|---|---|
| Original Residue | Conservative Substitutions |
| Ala | Ser |
| Arg | Gly; Gln; Lys |
| Asn | Gln; His |
| Asp | Glu |
| Cys | Ser |
| Gln | Asn; Lys |
| Glu | Asp |
| Gly | Ala |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

TABLE 12
Amino Acid Abbreviations

| Amino Acid | Abbreviations |
|---|---|
| Alanine | Ala (A) |
| Allosoleucine | AIle |
| Arginine | Arg (R) |
| Asparagine | Asn (N) |
| Aspartic Acid | Asp (D) |
| Cysteine | Cys (C) |
| Glutamic Acid | Glu (E) |
| Glutamine | Gln (Q) |
| Glycine | Gly (G) |
| Histidine | His (H) |
| Isolelucine | Ile (I) |
| Leucine | Leu (L) |
| Lysine | Lys (K) |
| Phenylalanine | Phe (F) |
| Praline | Pro (P) |
| Pyroglutamic Acid | PGlu (U) |
| Serine | Ser (S) |
| Threonine | Thr (T) |
| Tyrosine | Tyr (Y) |
| Tryptophan | Trp (W) |
| Valine | Val (V) |

It is understood that one way to define the variants and derivatives of the disclosed proteins herein is to define them in terms of homology/identity to specific known sequences. Specifically disclosed are variants of synthetic Apo E mimetics and other proteins or peptides herein disclosed which have at least, 70% or at least 75% or at least 80% or at least 85% or at least 90% or at least 95% homology to the synthetic Apo E mimetics specifically recited herein. Those of skill in the art readily understand how to determine the homology of two proteins.

The polypeptides can be modified by either natural processes, such as posttranslational processing, or by chemical modification techniques which are well known in the art. Modifications can occur anywhere in the polypeptide, including the peptide backbone, the amino acid side-chains and the amino or carboxyl termini. The same type of modification can be present in the same or varying degrees at several sites in a given polypeptide. Also, a given polypeptide can have many types of modifications. Modifications include, without limitation, acetylation, acylation, ADP-ribosylation, amidation, covalent cross-linking or cyclization, covalent attachment of flavin, covalent attachment of a heme moiety, covalent attachment of a nucleotide or nucleotide derivative, covalent attachment of a lipid or lipid derivative, covalent attachment of a phosphytidylinositol, disulfide bond formation, demethylation, formation of cysteine or pyroglutamate, formylation, gamma-carboxylation, glycosylation, GPI anchor formation, hydroxylation, iodination, methylation, yristolyation, oxidation, pergylation, proteolytic processing, phosphorylation, prenylation, racemization, selenoylation, sulfation, and transfer-RNA mediated addition of amino acids to protein such as arginylation. (See Proteins—Structure and Molecular Properties 2nd Ed., T. E. Creighton, W.H. Freeman and Company, New York (1993); Posttranslational Covalent Modification of Proteins, B. C. Johnson, Ed., Academic Press, New York, pp. 1-12 (1983)).

Variants can also include peptidomimetics. As used herein, “peptidomimetic” means a mimetic of a function of a protein which includes some alteration of the normal peptide chemistry. Peptidomimetics typically are short sequences of amino acids that in biological properties, mimic one or more function(s) of a particular protein. Peptide analogs enhance some property of the original peptide, such as increases stability, increased efficacy, enhanced delivery, increased half-life, etc. Methods of making peptidomimetics based upon a known polypeptide sequence is described, for example, in U.S. Pat. Nos. 5,631,280; 5,612,895; and 5,579,250. Use of peptidomimetics can involve the incorporation of a non-amino acid residue with non-amide linkages at a given position. One embodiment of the present invention is a peptidomimetic wherein the compound has a bond, a peptide backbone or an amino acid component replaced with a suitable mimic. Some non-limiting examples of unnatural amino acids which may be suitable amino acid mimics include β-alanine, L-α-amino butyric acid, L-γ-amino butyric acid, L-α-amino isobutyric acid, L-ε-amino caproic acid, 7-amino heptanoic acid, L-aspartic acid, L-glutamic acid, N-ε-Boc-N-α-CBZ-L-lysine, N-ε-Boc-N-α-Fmoc-L-lysine, L-methionine sulfone, L-norleucine, L-norvaline, N-α-Boc-N-δCBZ-L-ornithine, N-δ-Boc-N-α-CBZ-L-ornithine, Boc-p-nitro-L-phenylalanine, Boc-hydroxyproline, and Boc-L-thioproline.

v. Nucleic Acids

As this specification discusses various peptide sequences it is understood that the nucleic acids that can encode those polypeptide sequences are also disclosed. This would include all degenerate sequences related to a specific polypeptide sequence, i.e. all nucleic acids having a sequence that encodes one particular polypeptide sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed polypeptide sequences.

vi. Blocking/Protecting Groups and D Residues

While the various compositions described herein may be shown with no protecting groups, in certain embodiments (e.g., particularly for oral administration), they can bear one, two, three, four, or more protecting groups. The protecting groups can be coupled to the C- and/or N-terminus of the peptide(s) and/or to one or more internal residues comprising the peptide(s) (e.g., one or more R-groups on the constituent amino acids can be blocked). Thus, for example, in certain embodiments, any of the peptides described herein can bear, e.g., an acetyl group protecting the amino terminus and/or an amide group protecting the carboxyl terminus. One example of such a “dual protected peptide” is Ac-LRKLRKRLLRDWLKAFYDKVAEKLKEAF-NH₂ (SEQ ID NO:1) with blocking groups), either or both of these protecting groups can be eliminated and/or substituted with another protecting group as described herein. Without being bound by a particular theory, it was a discovery of this invention that blockage, particularly of the amino and/or carboxyl termini of the subject peptides of this invention can improve oral delivery and can also increase serum half-life.

A wide number of protecting groups are suitable for this purpose. Such groups include, but are not limited to acetyl, amide, and alkyl groups with acetyl and alkyl groups being particularly preferred for N-terminal protection and amide groups being preferred for carboxyl terminal protection. For example, the protecting groups can include, but are not limited to alkyl chains as in fatty acids, propeonyl, formyl, and others. Carboxyl protecting groups include amides, esters, and ether-forming protecting groups can also be used. For example, an acetyl group can be used to protect the amino terminus and an amide group can be used to protect the carboxyl terminus. These blocking groups enhance the helix-forming tendencies of the peptides. Additional blocking groups include alkyl groups of various lengths, e.g., groups having the formula: CH₃(CH₂)ₙCO where n ranges from about 1 to about 20, preferably from about 1 to about 16 or 18, more preferably from about 3 to about 13, and most preferably from about 3 to about 10.

Additionally, the protecting groups include, but are not limited to alkyl chains as in fatty acids, propeonyl, formyl, and others. For example, carboxyl protecting groups can include amides, esters, and ether-forming protecting groups. These blocking groups can enhance the helix-forming tendencies of the peptides. Blocking groups can include alkyl groups of various lengths, e.g., groups having the formula: CH₃(CH₂)ₙCO where n ranges from about 3 to about 20, preferably from about 3 to about 16, more preferably from about 3 to about 13, and most preferably from about 3 to about 10.

Other protecting groups include, but are not limited to Fmoc, t-butoxycarbonyl (t-BOC), 9-fluoreneacetyl group, 1-fluorenecarboxylic group, 9-fluorenecarboxylic group, 9-fluorenone-1-carboxylic group, benzyloxycarbonyl, Xanthyl (Xan), Trityl (Trt), 4-methyltrityl (Mtt), 4-methoxytrityl (Mmt), 4-methoxy-2,3,6-trimethyl-benzenesulphonyl (Mtr), Mesitylene-2-sulphonyl (Mts), 4,4-dimethoxybenzhydryl (Mbh), Tosyl (Tos), 2,2,5,7,8-pentamethyl chroman-6-sulphonyl (Pmc), 4-methylbenzyl (MeBzl), 4-methoxybenzyl (MeOBzl), Benzyloxy (BzlO), Benzyl (Bzl), Benzoyl (Bz), 3-nitro-2-pyridinesulphenyl (Npys), 1-(4,4-dimenthyl-2,6-diaxocyclohexylidene)ethyl (Dde), 2,6-dichlorobenzyl (2,6-DiCl-Bzl), 2-chlorobenzyloxycarbonyl (2-Cl—Z), 2-bromobenzyloxy-carbonyl (2-Br—Z), Benzyloxymethyl (Bom), cyclohexyloxy (cHxO), t-butoxymethyl (Bum), t-butoxy (tBuO), t-Butyl (tBu), Acetyl (Ac), and Trifluoroacetyl (TFA).

Protecting/blocking groups are well known to those of skill as are methods of coupling such groups to the appropriate residue(s) comprising the peptides of this invention (see, e.g., Greene et al., (1991) Protective Groups in Organic Synthesis, 2nd ed., John Wiley & Sons, Inc. Somerset, N.J.). For example, acetylation can be accomplished during the synthesis when the peptide is on the resin using acetic anhydride. Amide protection can be achieved by the selection of a proper resin for the synthesis.

The compositions disclosed herein can also comprise one or more D-form (dextro rather than levo) amino acids as described herein. For example, at least two enantiomeric amino acids, at least 4 enantiomeric amino acids or at least 8 or 10 enantiomeric amino acids can be in the “D” form amino acids. Additionally, every other, or even every amino acid (e.g., every enantiomeric amino acid) of the peptides described herein is a D-form amino acid.

Additionally, at least 50% of the enantiomeric amino acids can be “D” form, at least 80% of the enantiomeric amino acids are “D” form, at least 90%, or even all of the enantiomeric amino acids can be in the “D” form amino acids.

### C. Methods of Treating

Methods of treating atherosclerosis, peripheral or cerebral vascular disease by administering an effective amount of an Apo E mimetic for at least one treatment cycle followed by a rest phase are provided. Thus, the disclosed methods involve administering an Apo E mimetic using one or more of the disclosed dosing regimens. Thus, any of the disclosed treatment cycles or rest phases can be used in the disclosed methods. The methods disclosed herein can allow for prolonged therapeutic effects even in the absence of the therapeutic. The disclosed methods can include the administration of an effective amount of Apo E mimetic. The effective amount of an Apo E mimetic can be an amount that allows for sustained therapeutic effects after the Apo E mimetic has been withdrawn.

Disclosed herein are methods of treating atherosclerosis comprising administering to a subject an effective amount of an Apo E mimetic for at least one treatment cycle, wherein the treatment cycle comprises administering an effective amount of an Apo E mimetic to allow for a sustained therapeutic effect after withdrawal of the Apo E mimetic, wherein the treatment cycle is followed by a rest phase, wherein Apo E mimetic is not administered during the rest phase.

Disclosed herein are methods of treating atherosclerosis comprising administering to a subject an effective amount of an Apo E mimetic for at least one treatment cycle, wherein the treatment cycle comprises administering an effective amount of an Apo E mimetic to allow for a sustained therapeutic effect after withdrawal of the Apo E mimetic, wherein the treatment cycle is followed by a rest phase, wherein Apo E mimetic is not administered during the rest phase, wherein the rest phase is at least four weeks.

Disclosed herein are methods of treating atherosclerosis comprising administering to a subject an effective amount of an Apo E mimetic for at least one treatment cycle, wherein the treatment cycle comprises administering an effective amount of an Apo E mimetic to allow for a sustained therapeutic effect after withdrawal of the Apo E mimetic, wherein the treatment cycle is followed by a rest phase, wherein Apo E mimetic is not administered during the rest phase, further comprising a second treatment cycle after the rest phase. The second treatment cycle can be administered after a four week rest phase or one year from the beginning of the initial treatment cycle.

Disclosed herein are methods of treating atherosclerosis comprising administering to a subject an effective amount of an Apo E mimetic for at least one treatment cycle, wherein the treatment cycle comprises administering an effective amount of an Apo E mimetic to allow for a sustained therapeutic effect after withdrawal of the Apo E mimetic, wherein the treatment cycle is followed by a rest phase, wherein Apo E mimetic is not administered during the rest phase, wherein an atherosclerosis therapeutic other than an Apo E mimetic is administered during the rest phase. The atherosclerosis therapeutic other than an Apo E mimetic can be a conventional lipid lowering therapy and the conventional lipid lowering therapy can be a statin.

Disclosed herein are methods of treating atherosclerosis comprising administering to a subject an effective amount of an Apo E mimetic for at least one treatment cycle, wherein the treatment cycle comprises administering an effective amount of an Apo E mimetic to allow for a sustained therapeutic effect after withdrawal of the Apo E mimetic, wherein the Apo E mimetic consists of the amino acid sequence LRKLRKRLLRDWLKAFYDKVAEKLKEAF (SEQ ID NO:1), wherein the treatment cycle is followed by a rest phase, wherein Apo E mimetic is not administered during the rest phase.

Disclosed herein are methods of treating atherosclerosis comprising administering to a subject an effective amount of an Apo E mimetic for at least one treatment cycle, wherein the treatment cycle comprises administering an effective amount of an Apo E mimetic to allow for a sustained therapeutic effect after withdrawal of the Apo E mimetic, wherein the treatment cycle is followed by a rest phase, wherein Apo E mimetic is not administered during the rest phase, wherein the treatment cycle comprises administration of an effective amount of an Apo E mimetic once a week for three months.

Disclosed herein are methods of treating peripheral vascular disease comprising administering to a subject an effective amount of an Apo E mimetic for at least one treatment cycle, wherein the treatment cycle comprises administering an effective amount of an Apo E mimetic to allow for a sustained therapeutic effect after withdrawal of the Apo E mimetic, wherein the treatment cycle is followed by a rest phase, wherein Apo E mimetic is not administered during the rest phase.

Peripheral vascular disease (PVD) can also refer to peripheral artery disease (PAD) or peripheral artery occlusive disease (PAOD) or peripheral obliterative arteriopathy. Peripheral vascular disease refers to the obstruction of large arteries not within the coronary, aortic arch vasculature, or brain. PVD can result from atherosclerosis, inflammatory processes leading to stenosis, an embolism, orthrombus formation. PVD can cause either acute or chronic ischemia (lack of blood supply). PVD can also refer to atherosclerotic blockages found in the lower extremity

Peripheral artery occlusive disease is commonly divided in the Fontaine stages, introduced by René Fontaine in 1954 for ischemia. The Fontain stages are provide below in the following Table 13.

TABLE 13

| Fontain Stage | Substage | Description |
|---|---|---|
| Stage I | | Asymptomatic, incomplete |
| | | blood vessel obstruction |
| Stage II | | Mild claudication |
| | Stage IIA | Claudication at a distance of |
| | | greater than 200 metres |
| | Stage IIB | Claudication distance of |
| | | less than 200 metres |
| Stage III | | Rest pain, mostly in the feet |
| Stage IV | | Necrosis and/or gangrene of |
| | | the limb |

Peripheral artery occlusive disease can also refer to a classification designed by Rutherford which consists of three grades and six categories. The Rutherford stages are Provide below in the following Table 14.

TABLE 14

| Rutherford Stage | Description |
|---|---|
| Category 0 | Asymptomatic |
| Category 1 | Mild claudication |
| Category 2 | Moderate claudication |
| Category 3 | Severe claudication |
| Category 4 | Rest pain |
| Category 5 | Minor tissue loss; Ischemic ulceration not |
| | exceeding ulcer of the digits of the foot |
| Category 6 | Major tissue loss; Severe ischemic ulcers |
| | or frank gangrene |

Disclosed herein are methods of treating peripheral vascular disease comprising administering to a subject an effective amount of an Apo E mimetic for at least one treatment cycle, wherein the treatment cycle comprises administering an effective amount of an Apo E mimetic to allow for a sustained therapeutic effect after withdrawal of the Apo E mimetic, wherein the treatment cycle is followed by a rest phase, wherein Apo E mimetic is not administered during the rest phase, wherein the rest phase is at least four weeks.

Disclosed herein are methods of treating peripheral vascular disease comprising administering to a subject an effective amount of an Apo E mimetic for at least one treatment cycle, wherein the treatment cycle comprises administering an effective amount of an Apo E mimetic to allow for a sustained therapeutic effect after withdrawal of the Apo E mimetic, wherein the treatment cycle is followed by a rest phase, wherein Apo E mimetic is not administered during the rest phase, further comprising a second treatment cycle after the rest phase. The second treatment cycle can be administered after a four week rest phase or one year from the beginning of the initial treatment cycle.

Disclosed herein are methods of treating peripheral vascular disease comprising administering to a subject an effective amount of an Apo E mimetic for at least one treatment cycle, wherein the treatment cycle comprises administering an effective amount of an Apo E mimetic to allow for a sustained therapeutic effect after withdrawal of the Apo E mimetic, wherein the treatment cycle is followed by a rest phase, wherein Apo E mimetic is not administered during the rest phase, wherein a peripheral vascular disease therapeutic other than an Apo E mimetic is administered during the rest phase. The atherosclerosis therapeutic other than an Apo E mimetic can be a conventional lipid lowering therapy and the conventional lipid lowering therapy can be one or more of the following: a statin, an antiplatelet drus, aspirin, clopidogrel, Cilostazol, or pentoxifylline. The atherosclerosis therapeutic other than an Apo E mimetic can also include surgical intervention, smoking cessation, management of diabetes, management of hypertension management of cholesterol, exercise, Angioplasty (PTA or percutaneous transluminal angioplasty), plaque excision, bypass grafting, sympathectomy, amputation, or thrombolysis.

Disclosed herein are methods of treating peripheral vascular disease comprising administering to a subject an effective amount of an Apo E mimetic for at least one treatment cycle, wherein the treatment cycle comprises administering an effective amount of an Apo E mimetic to allow for a sustained therapeutic effect after withdrawal of the Apo E mimetic, wherein the Apo E mimetic consists of the amino acid sequence LRKLRKRLLRDWLKAFYDKVAEKLKEAF (SEQ ID NO:1), wherein the treatment cycle is followed by a rest phase, wherein Apo E mimetic is not administered during the rest phase.

Disclosed herein are methods of treating peripheral vascular disease comprising administering to a subject an effective amount of an Apo E mimetic for at least one treatment cycle, wherein the treatment cycle comprises administering an effective amount of an Apo E mimetic to allow for a sustained therapeutic effect after withdrawal of the Apo E mimetic, wherein the treatment cycle is followed by a rest phase, wherein Apo E mimetic is not administered during the rest phase, wherein the treatment cycle comprises administration of an effective amount of an Apo E mimetic once a week for three months.

Disclosed herein are methods of treating critical limb ischemia comprising administering to a subject an effective amount of an Apo E mimetic for at least one treatment cycle, wherein the treatment cycle comprises administering an effective amount of an Apo E mimetic to allow for a sustained therapeutic effect after withdrawal of the Apo E mimetic, wherein the treatment cycle is followed by a rest phase, wherein Apo E mimetic is not administered during the rest phase.

Critical limb ischemia (CLI) is the most severe form of peripheral arterial disease (PAD) caused by chronic inflammatory processes associated with atherosclerosis that result in markedly reduced blood flow to the legs, feet and hands. There are an estimated 10-12 million people with PAD in the United States, and over 1 million people with CLI.

Disclosed herein are methods of treating critical limb ischemia comprising administering to a subject an effective amount of an Apo E mimetic for at least one treatment cycle, wherein the treatment cycle comprises administering an effective amount of an Apo E mimetic to allow for a sustained therapeutic effect after withdrawal of the Apo E mimetic, wherein the treatment cycle is followed by a rest phase, wherein Apo E mimetic is not administered during the rest phase, wherein the rest phase is at least four weeks.

Disclosed herein are methods of treating critical limb ischemia comprising administering to a subject an effective amount of an Apo E mimetic for at least one treatment cycle, wherein the treatment cycle comprises administering an effective amount of an Apo E mimetic to allow for a sustained therapeutic effect after withdrawal of the Apo E mimetic, wherein the treatment cycle is followed by a rest phase, wherein Apo E mimetic is not administered during the rest phase, further comprising a second treatment cycle after the rest phase. The second treatment cycle can be administered after a four week rest phase or one year from the beginning of the initial treatment cycle.

Disclosed herein are methods of treating critical limb ischemia comprising administering to a subject an effective amount of an Apo E mimetic for at least one treatment cycle, wherein the treatment cycle comprises administering an effective amount of an Apo E mimetic to allow for a sustained therapeutic effect after withdrawal of the Apo E mimetic, wherein the treatment cycle is followed by a rest phase, wherein Apo E mimetic is not administered during the rest phase, wherein an peripheral vascular disease therapeutic other than an Apo E mimetic is administered during the rest phase. The critical limb ischemia therapeutic other than an Apo E mimetic can be a conventional lipid lowering therapy and the conventional lipid lowering therapy can be one or more of the following: a statin, an antiplatelet drus, aspirin, clopidogrel, Cilostazol, or pentoxifylline. The atherosclerosis therapeutic other than an Apo E mimetic can also include surgical intervention, smoking cessation, management of diabetes, management of hypertension management of cholesterol, exercise, Angioplasty (PTA or percutaneous transluminal angioplasty), plaque excision, bypass grafting, sympathectomy, amputation, or thrombolysis.

Disclosed herein are methods of treating critical limb ischemia comprising administering to a subject an effective amount of an Apo E mimetic for at least one treatment cycle, wherein the treatment cycle comprises administering an effective amount of an Apo E mimetic to allow for a sustained therapeutic effect after withdrawal of the Apo E mimetic, wherein the Apo E mimetic consists of the amino acid sequence LRKLRKRLLRDWLKAFYDKVAEKLKEAF (SEQ ID NO:1), wherein the treatment cycle is followed by a rest phase, wherein Apo E mimetic is not administered during the rest phase.

Disclosed herein are methods of treating critical limb ischemia comprising administering to a subject an effective amount of an Apo E mimetic for at least one treatment cycle, wherein the treatment cycle comprises administering an effective amount of an Apo E mimetic to allow for a sustained therapeutic effect after withdrawal of the Apo E mimetic, wherein the treatment cycle is followed by a rest phase, wherein Apo E mimetic is not administered during the rest phase, wherein the treatment cycle comprises administration of an effective amount of an Apo E mimetic once a week for three months.

Any of the disclosed Apo E mimetics can be used in these methods. For example, the Apo E mimetic can be hE18A (LRKLRKRLLRDWLKAFYDKVAEKLKEAF; SEQ ID NO:1), in particular the Apo E mimetic can be the hE18A peptide that has an acetyl group on the amino terminus and an amide group at the carboxyl terminus.

The disclosed methods of treating can occur at different times depending on the subject. In particular, treatment can occur in a subject considered to be of high, or high residual risk of a cardio- or cerebrovascular event. In one instance, the treatment can be initiated after a subject is stabilized following an acute coronary event. In one instance, the treatment can be initiated immediately after the acute coronary event, or 3, 6, 9, or 12 months after the acute coronary event. The treatment can be initiated following acute interventional cardiology procedures such as coronary artery bypass surgery (CABG), percutaneous coronary intervention (angioplasty, PCI), or implant of a stent into a coronary artery. Subjects considered as high risk can be those individuals that have homozygous familial hypercholesterolemia (FH), severe refractory FH, diabetes or an individual following acute coronary syndrome (ACS). In high risk subjects, treatment can be extended.

1. Treatment Cycle

The treatment cycle, as previously described with respect to the dosing regimens, can vary in length of time. The treatment cycle can be at least four weeks but can last up to six months. In one instance, the disclosed methods have a treatment cycle that involves the administration of an effective amount of an Apo E mimetic once a week for one month (four weeks), three months (12 weeks) or six months (24 weeks). A treatment cycle can include the administration of Apo E mimetic once, twice or three times a week. In some aspects, the Apo E mimetic can be administered daily. In some aspects, the Apo E mimetic can be administered once every two weeks or even once a month. In some instances, the Apo E mimetic can be administered every two weeks for 4, 6, 8, 10, 12, 14, 16, 18, 20, 22, or 24 weeks. Each treatment cycle can include an established length of time for administration as well as an established dosing schedule during that time frame.

The methods can further include a second treatment cycle after the rest phase. In one aspect, the second treatment cycle can be administered after a four week rest phase. In another aspect, the second treatment cycle can be administered at least one year from the beginning of the initial treatment cycle.

2. Rest Phase

The rest phase, as previously described with regards to the dosing regimen, can be at least four weeks but can last for several years. Apo E mimetic is not administered during the rest phase.

The length of the rest phase is dependent on how long the sustained therapeutic effects of the Apo E mimetic administered during the treatment cycle last. In some instances the rest phase can be at least 1, 2, 3, 4, 5, 6, 7, 8, 9, 10, 11, or 12 months. In some instances the rest phase can be at least 1, 2, 3, 4, 5, 6, 7, 8, 9, or 10 years. For example, the rest phase can be at least four weeks (one month).

In one aspect, the rest phase can be decreased or extended depending on the dose of Apo E mimetic administered during the treatment cycle. For example, the rest phase can be extended if the dose of Apo E mimetic during the treatment cycle is increased. The length of the rest phase can also vary based on the length of the treatment cycle. For instance, if a subject receives a certain dose of Apo E mimetic once a week for three months then the rest phase may be shorter than a subject that receives the same dose of Apo E mimetic once a week for six months.

Although an Apo E mimetic is not administered during the rest phase, an atherosclerosis therapeutic other than an Apo E mimetic can be administered during the rest phase. The atherosclerosis therapeutic other than an Apo E mimetic can be a conventional lipid lowering therapy, such as a statin, or bile acid sequestrant, and/or a therapeutic such as a PCSK9 inhibitor, a VLDL synthesis inhibitor and/or a CETP inhibitor.

3. Atherosclerosis

The combination of LDL accumulation in a vessel wall and an inflammatory response to the LDL's is responsible for initiating atherosclerosis. The LDL within the vessel wall becomes oxidized which damages the vessel wall and triggers an immune response. Immune cells, such as macrophages, are not able to process the oxidized-LDL and eventually rupture which leads to more oxidized cholesterol in the artery wall. This cycle continues which causes more and more damage to the vessel walls. The increase in cholesterol leads to plaques which ultimately results in hardening and narrowing of the vessel wall. The disclosed methods are useful for treating atherosclerosis.

4. Delivery

In the methods described herein, administration or delivery of the Apo E mimetics can be via a variety of mechanisms. As defined above, disclosed herein are dosing regimens and methods of using those dosing regimens to treat atherosclerosis. The dosing regimens and methods include compositions containing any one or more of the polypeptides or nucleic acids described herein that can also include a carrier such as a pharmaceutically acceptable carrier. For example, disclosed are pharmaceutical compositions, comprising the Apo E mimetics disclosed herein, and a pharmaceutically acceptable carrier.

The disclosed Apo E mimetics can be in solution or in suspension (for example, incorporated into microparticles, liposomes, or cells). These compositions can be targeted to a particular cell type via antibodies, receptors, or receptor ligands. One of skill in the art knows how to make and use such targeting agents with the disclosed compositions. A targeting agent can be a vehicle such as an antibody conjugated liposomes; receptor mediated targeting of DNA through cell specific ligands, and highly specific retroviral targeting of cells in vivo. Any such vehicles can be part of the compositions herein. For example, targeting agents that direct the Apo E mimetic to the blood vessel walls can be included in the compositions.

Any suitable route of administration can be used for the disclosed compositions. Suitable routes of administration can, for example, include topical, enteral, local, systemic, or parenteral. For example, administration can be epicutaneous, inhalational, enema, conjunctival, eye drops, ear drops, alveolar, nasal, intranasal, enteral, oral, intraoral, transoral, intestinal, rectal, intrarectal, transrectal, injection, infusion, intravenous, intraarterial, intramuscular, intracerebral, intraventricular, intracerebroventricular, intracardiac, subcutaneous, intraosseous, intradermal, intrathecal, intraperitoneal, intravesical, intracavernosal, intramedullar, intraocular, intracranial, transdermal, transmucosal, transnasal, inhalational, intracisternal, epidural, peridural, intravitreal, etc. The disclosed compositions can be used in and with any other therapy.

The compositions described herein can comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidyl (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, Pa. 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

Pharmaceutical compositions may also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like. The pharmaceutical composition may be administered in a number of ways depending on whether local or systemic treatment is desired, and on the area to be treated.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium choloride solution, Ringer's dextrose, dextrose and sodium choloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

Unlike typical peptide formulations, the peptides of this invention comprising D-form amino acids can be administered, even orally, without protection against proteolysis by stomach acid, etc. Nevertheless, in certain embodiments, peptide delivery can be enhanced by the use of protective excipients. This is typically accomplished either by complexing the polypeptide with a composition to render it resistant to acidic and enzymatic hydrolysis or by packaging the polypeptide in an appropriately resistant carrier such as a liposome. Means of protecting polypeptides for oral delivery are well known in the art (see, e.g., U.S. Pat. No. 5,391,377 describing lipid compositions for oral delivery of therapeutic agents).

Elevated serum half-life can be maintained by the use of sustained-release protein “packaging” systems. Such sustained release systems are well known to those of skill in the art. In one preferred embodiment, the ProLease biodegradable microsphere delivery system for proteins and peptides (Tracy (1998) Biotechnol. Prog., 14: 108; Johnson et al. (1996) Nature Med. 2: 795; Herbert et al. (1998), Pharmaceut. Res. 15, 357) a dry powder composed of biodegradable polymeric microspheres containing the active agent in a polymer matrix that can be compounded as a dry formulation with or without other agents.

The ProLease microsphere fabrication process was specifically designed to achieve a high encapsulation efficiency while maintaining integrity of the active agent. The process consists of (i) preparation of freeze-dried drug particles from bulk by spray freeze-drying the drug solution with stabilizing excipients, (ii) preparation of a drug-polymer suspension followed by sonication or homogenization to reduce the drug particle size, (iii) production of frozen drug-polymer microspheres by atomization into liquid nitrogen, (iv) extraction of the polymer solvent with ethanol, and (v) filtration and vacuum drying to produce the final dry-powder product. The resulting powder contains the solid form of the active agents, which is homogeneously and rigidly dispersed within porous polymer particles. The polymer most commonly used in the process, poly(lactide-co-glycolide) (PLG), is both biocompatible and biodegradable.

Encapsulation can be achieved at low temperatures (e.g., −40° C.). During encapsulation, the protein is maintained in the solid state in the absence of water, thus minimizing water-induced conformational mobility of the protein, preventing protein degradation reactions that include water as a reactant, and avoiding organic-aqueous interfaces where proteins may undergo denaturation. A preferred process uses solvents in which most proteins are insoluble, thus yielding high encapsulation efficiencies (e.g., greater than 95%).

In another embodiment, one or more components of the solution can be provided as a “concentrate”, e.g., in a storage container (e.g., in a premeasured volume) ready for dilution, or in a soluble capsule ready for addition to a volume of water.

The foregoing formulations and administration methods are intended to be illustrative and not limiting. It will be appreciated that, using the teaching provided herein, other suitable formulations and modes of administration can be readily devised.

5. Combination Therapy

In one aspect of the disclosed methods, the Apo E mimetics can be administered alone or in combination with one or more additional therapeutic agents. The additional therapeutic agents are selected based on the disease or symptom to be treated. A description of the various classes of suitable pharmacological agents and drugs may be found in Goodman and Gilman, The Pharmacological Basis of Therapeutics, (11th Ed., McGraw-Hill Publishing Co.) (2005). For example, pharmaceutical compositions containing Apo E mimetics can be administered in combination with one or more known therapeutic agents for treating atherosclerosis. Therapeutic agents for treating atherosclerosis include, but are not limited to, cholesterol-lowering agents, blood pressure-lowering agents, blood thinning agents (i.e. medicines that prevent blood clots), anti-inflammatory agents, and anti-atherogenic agents. Examples of cholesterol-lowering agents include, but are not limited to, a cholesterol absorption inhibitor, a bile acid sequestrant, a fibrate, a PCSK9 inhibitor, a microsomal triglyceride transfer protein inhibitor, an apolipoprotein B synthesis inhibitor, or a CETP inhibitor.

In one aspect of the disclosed methods, the Apo E mimetics can be administered alone or in combination with one or more additional peripheral vascular disease therapeutic agents. For example, pharmaceutical compositions containing Apo E mimetics can be administered in combination with one or more known therapeutic agents for treating peripheral vascular disease. Therapeutic agents for treating peripheral vascular disease include, but are not limited to, cholesterol-lowering agents, blood pressure-lowering agents, blood thinning agents (i.e. medicines that prevent blood clots), anti-inflammatory agents, and anti-atherogenic agents. Examples of cholesterol-lowering agents include, but are not limited to, a cholesterol absorption inhibitor, a bile acid sequestrant, a fibrate, a PCSK9 inhibitor, a microsomal triglyceride transfer protein inhibitor, an apolipoprotein B synthesis inhibitor, or a CETP inhibitor.

In one aspect of the disclosed methods, the Apo E mimetics can be administered alone or in combination with one or more additional interventional therapies such as angioplasty, artherectomy or grafting.

In one aspect of the disclosed methods, the Apo E mimetics can be administered alone or in combination with one or more additional critical limb ischemia therapeutic agents. For example, pharmaceutical compositions containing Apo E mimetics can be administered in combination with one or more known therapeutic agents for treating critical limb ischemia. Therapeutic agents for treating critical limb ischemia include, but are not limited to, cholesterol-lowering agents, blood pressure-lowering agents, blood thinning agents (i.e. medicines that prevent blood clots), anti-inflammatory agents, and anti-atherogenic agents. Examples of cholesterol-lowering agents include, but are not limited to, a cholesterol absorption inhibitor, a bile acid sequestrant, a fibrate, a PCSK9 inhibitor, a microsomal triglyceride transfer protein inhibitor, an apolipoprotein B synthesis inhibitor, or a CETP inhibitor.

The critical limb ischemia therapeutic other than an Apo E mimetic can be a conventional lipid lowering therapy and the conventional lipid lowering therapy can be one or more of the following: a statin, an antiplatelet drus, aspirin, clopidogrel, Cilostazol, or pentoxifylline. The atherosclerosis therapeutic other than an Apo E mimetic can also include surgical intervention, smoking cessation, management of diabetes, management of hypertension management of cholesterol, exercise, Angioplasty (PTA or percutaneous transluminal angioplasty), plaque excision, bypass grafting, sympathectomy, amputation, or thrombolysis.

The Apo E mimetics can be administered in conjunction with or followed by any of the disclosed additional therapeutics. The treatments can be administered in conjunction with or followed by LDL apheresis.

The combination therapies can include administering the Apo E mimetic and an additional therapeutic agent during the treatment cycle of a dosing regimen. The combination therapies can also include administering the Apo E mimetic during the treatment cycle and an additional therapeutic agent during the rest phase.

### EXAMPLES

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

Those skilled in the art will recognize, or be able to ascertain using no more than routine experimentation, many equivalents to the specific embodiments of the method and compositions described herein. Such equivalents are intended to be encompassed by the following claims.

### A. Example 1

### Study One—Prolonged Effect of Apo E Mimetics (a Comparison of hE18A with mR18L, a Single Domain Cationic Peptide)

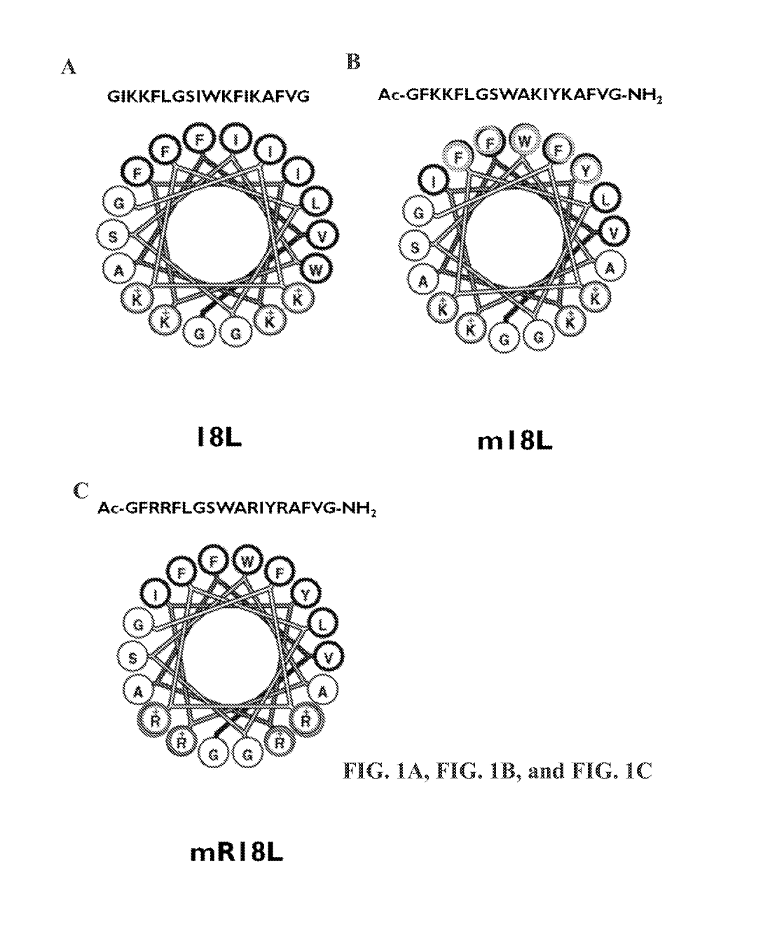

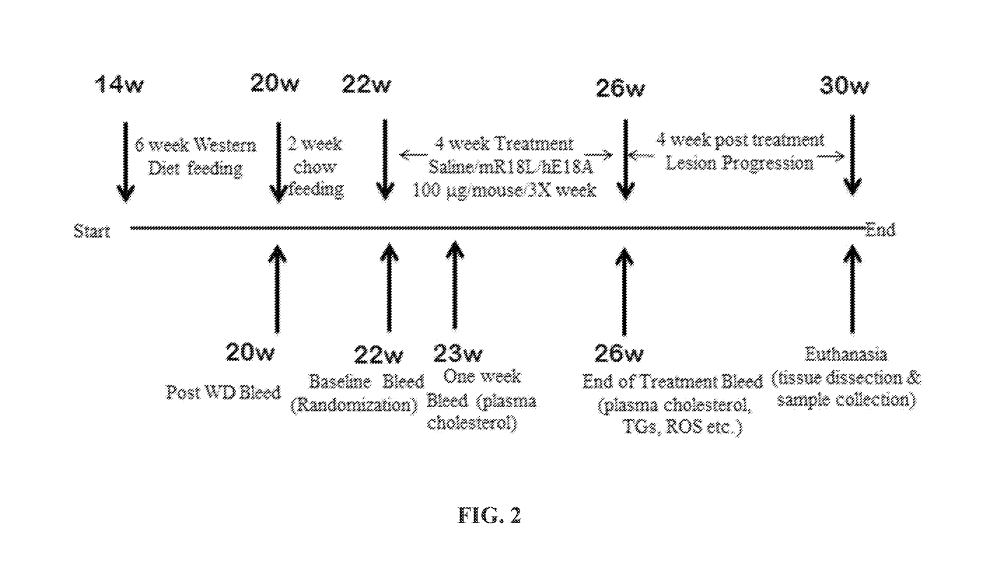

FIG. 1 shows the design of mR18L (Tytler et al. JBC, 268:22112-22118, 1993; Handattu et al. J. Lipid Res, 51:3491-3499, 2010). The experimental protocol was performed in Apo E null mice as shown in FIG. 2. The mice were fed a Western diet for 6 weeks to elevate cholesterol. The animals were then switched to a chow diet for two weeks after which they were treated with peptide or saline for 4 weeks. Treatment was then withdrawn for 4 weeks while the animals remained on a chow diet. After the 4 week post treatment phase, the mice were euthanized and tissue dissection and sample collection was performed.

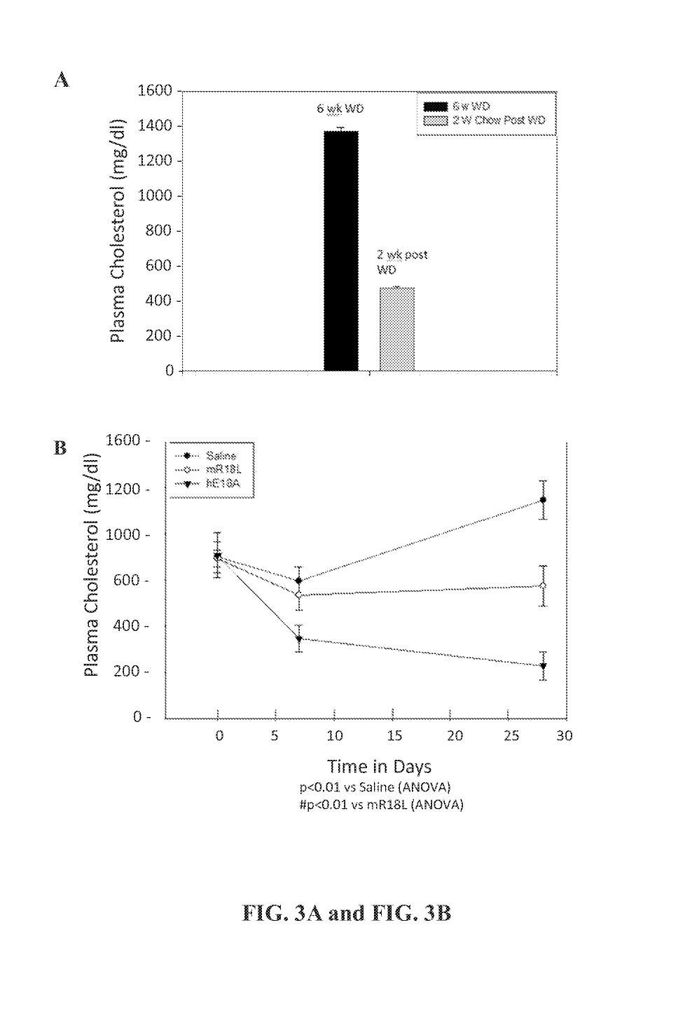

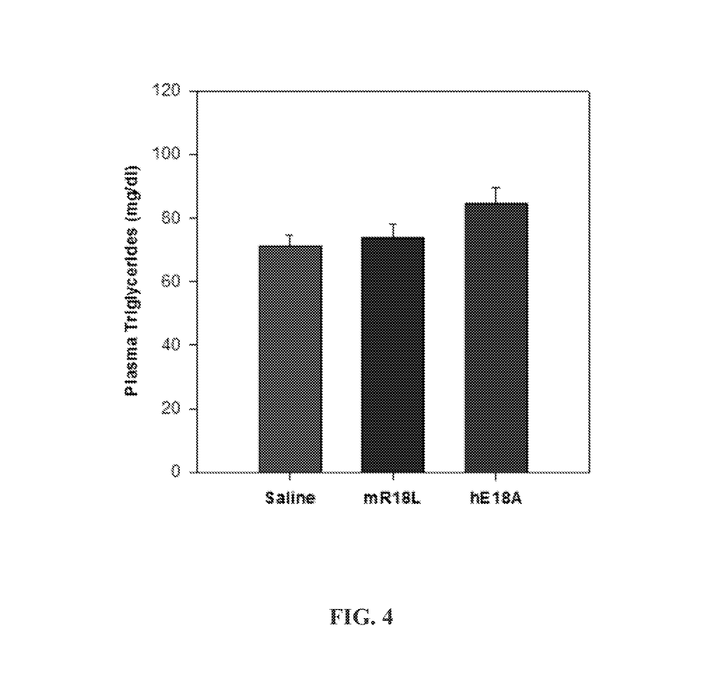

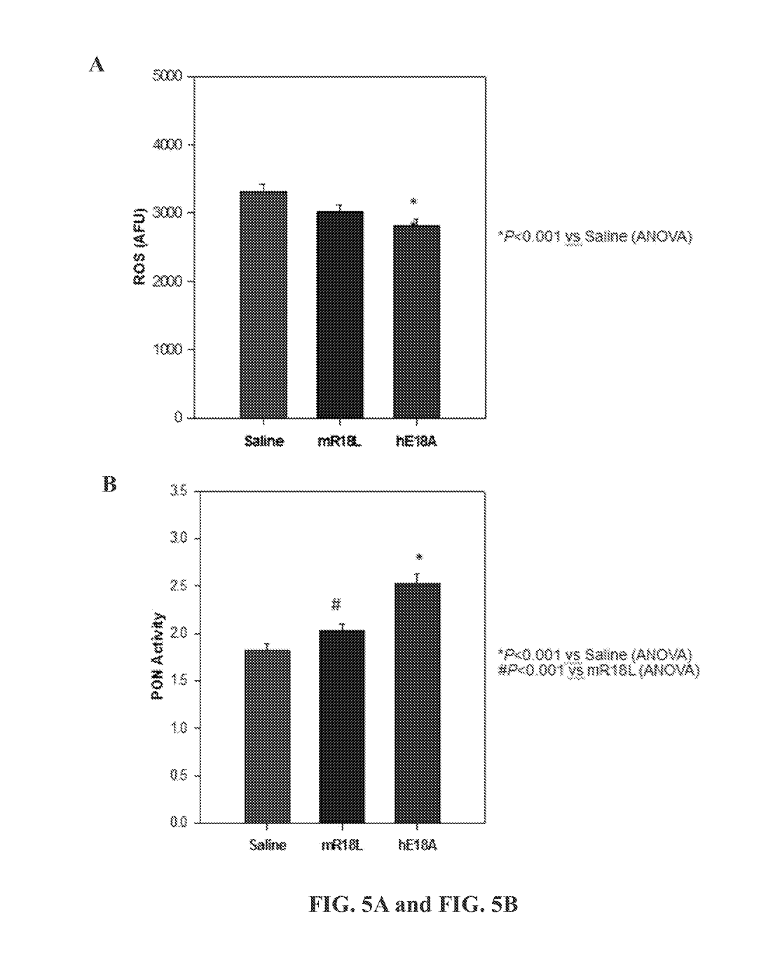

Blood was drawn after 4 weeks of treatment at week 26 and analyzed for cholesterol. At the end of treatment, hE18A was better in reducing plasma cholesterol levels in Apo E null mice compared to mR18L and saline (FIGS. 3A and 3B). Apo E mimetic peptide treatment did not change plasma triglyceride levels (FIG. 4). However, Apo E mimetic treatment did improve plasma marker of oxidative stress, such as ROS and PON activity. FIG. 5A shows a significant decrease in reactive oxygen species (ROS) in the hE18A treated mice compared to the saline treated mice. Because ROS have been implicated in the pathogenesis of atherosclerosis, the decrease in ROS by treatment with an Apo E mimetic is beneficial.

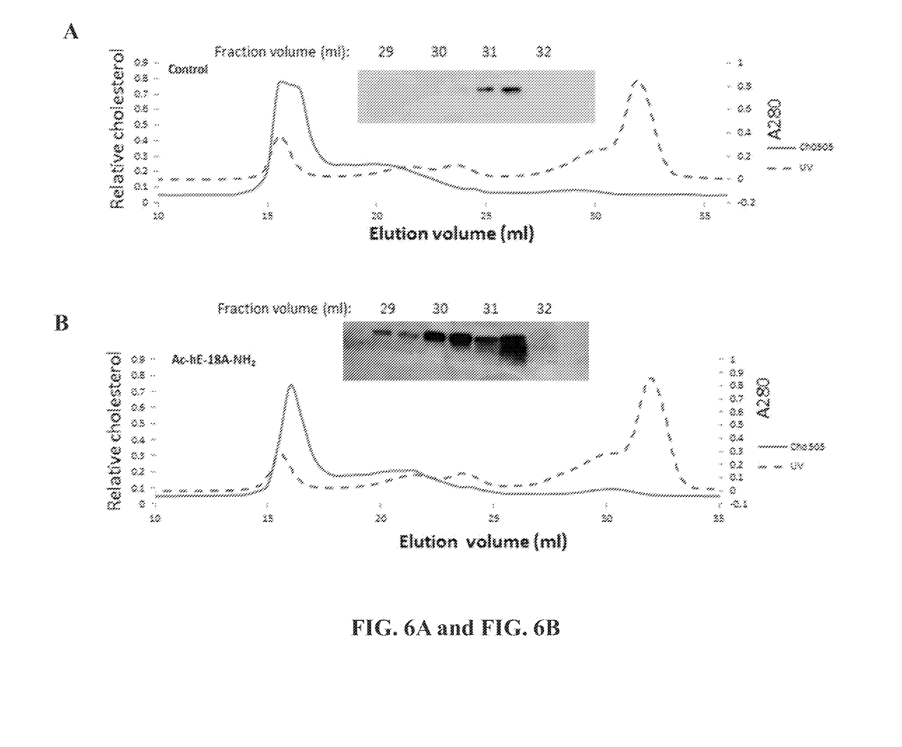

FIG. 5B shows a significant increase in paraoxonase (PON) activity in hE18A treated mice compared to saline and mR18L treated mice. The PON gene family has anti-atherosclerotic effects, such as preventing oxidation of LDLs. Thus, the increase in PON activity in hE18A treated mice is beneficial in the atherosclerosis animal model. FIGS. 6A and 6B further shows the dramatic increase in PON-1 levels in HDL fractions in Apo E null mice treated with Ac-hE18A-NH₂.

The mice were euthanized at week 30, which is four weeks post treatment with Apo E mimetic.

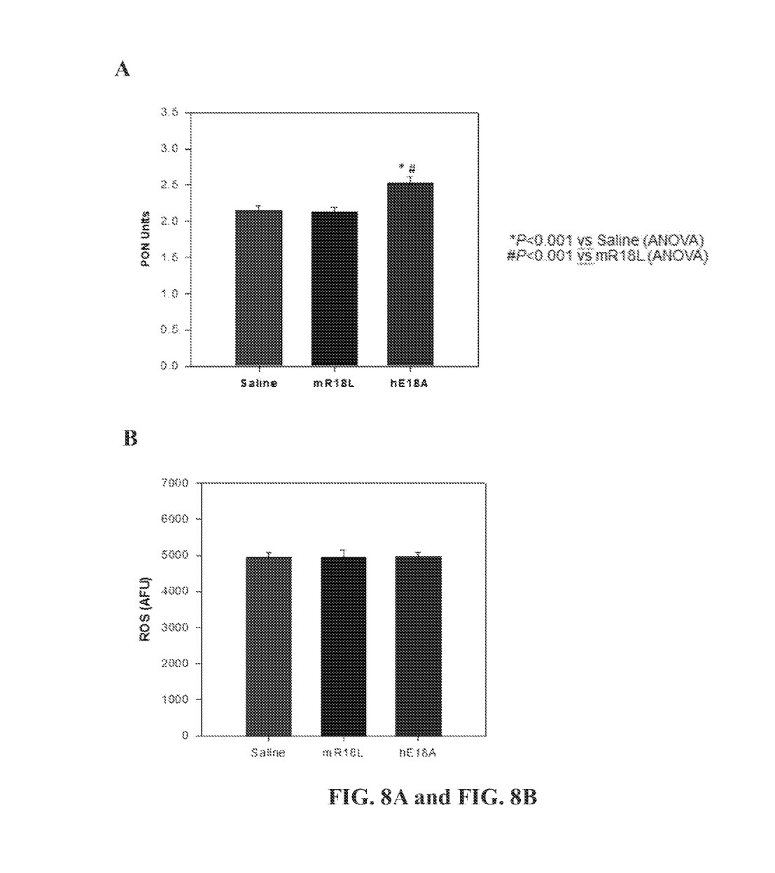

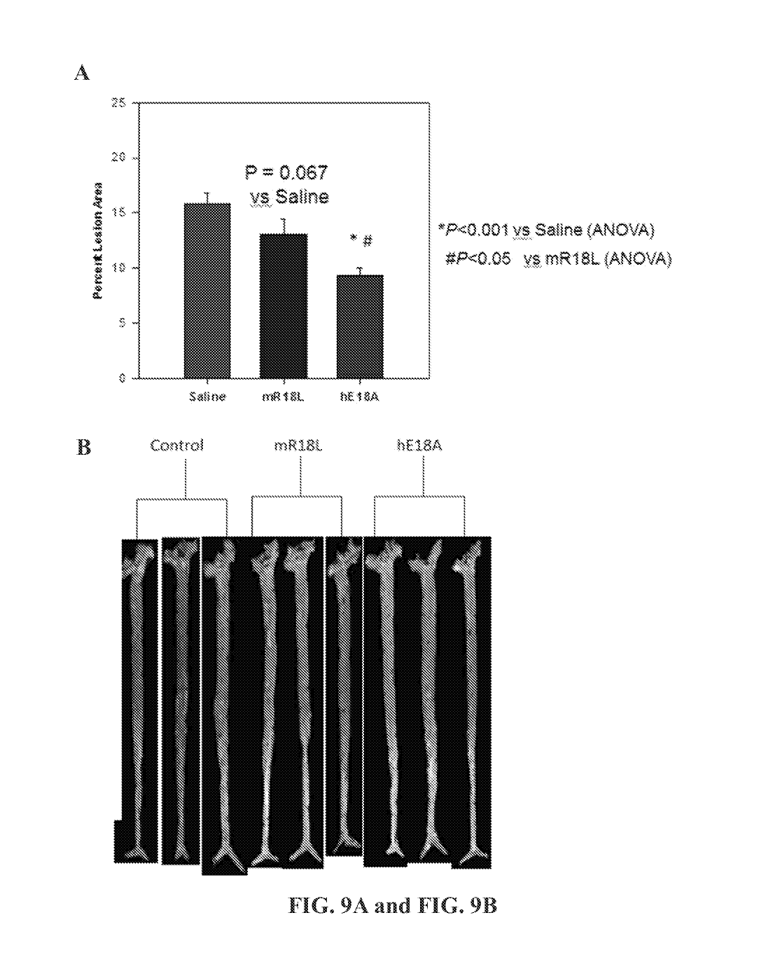

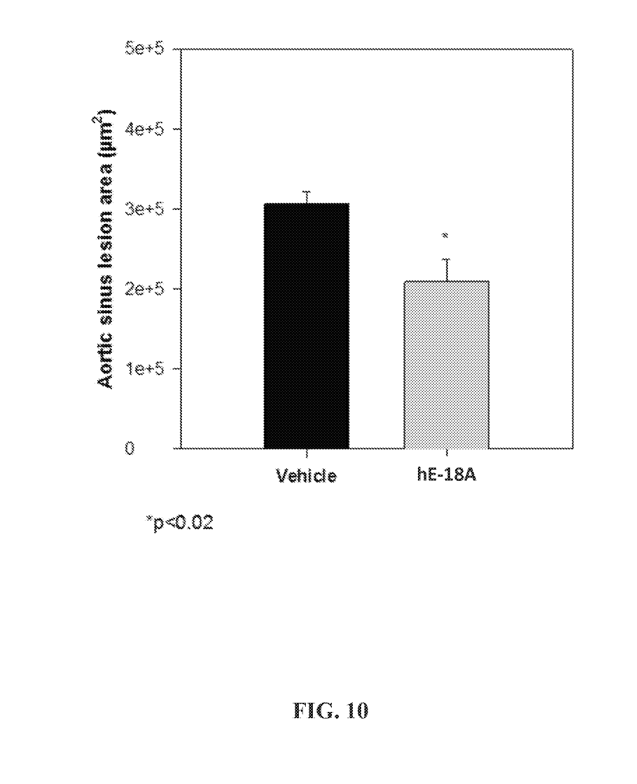

FIGS. 7 and 8B show that cholesterol levels and ROS levels, respectively, were not significantly different four weeks after the end of treatment period. However, PON-1 activity was still increased in the hE18A treated mice over the saline and mR18L treated mice 4 weeks post treatment (FIG. 8A). Importantly, the atherosclerotic lesion area was significantly reduced in the hE18A treated mice compared to the saline or mR18L treated mice (FIGS. 9A and 9B). Atherosclerotic lesion area in the aortic sinus was also significantly decreased by hE18A (FIG. 10), but not by mR18L. There was also a non-significant trend towards a decrease in macrophage coverage of the aortic sinus with hE18A (FIG. 11).

### B. Example 2

### Study Two—Late Intervention Study in Apo E −/− Mice

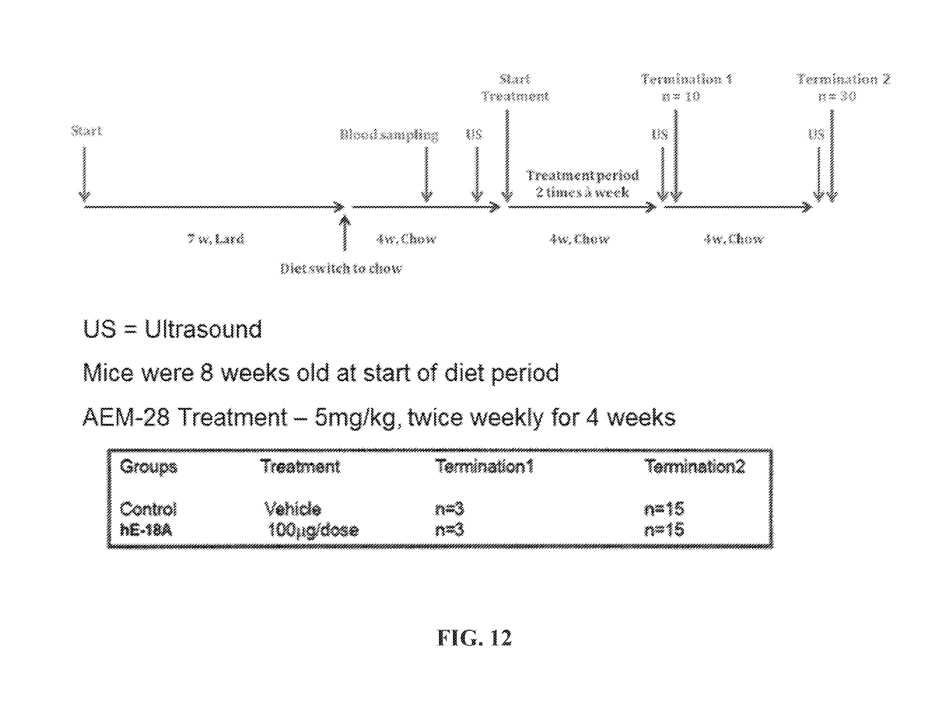

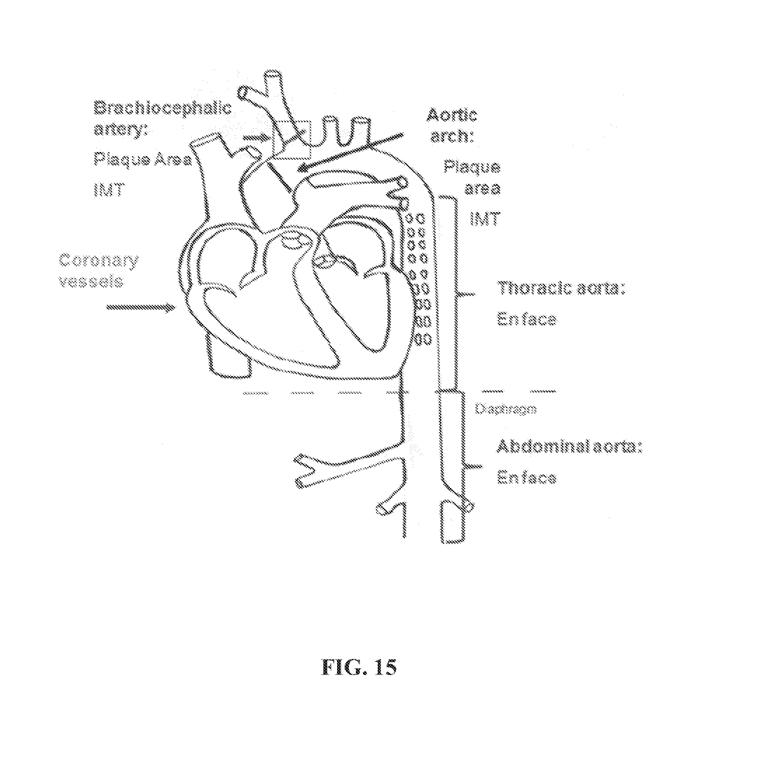

FIG. 12 shows the study design used to treat Apo E −/− mice. The mice were fed a lard diet for seven weeks and then were switched to a chow diet for four weeks. The mice were treated two times a week for four weeks and then treatment was withdrawn for four weeks. Termination 1 represents the time point immediately following termination of the Apo E mimetic treatment. Termination 2 represents the time point immediately following the four weeks of treatment withdrawal. Lipids were analyzed in the plasma (cholesterol, triglycerides) and in the liver (cholesterol esters, unesterified cholesterol, triglycerides). A cardiovascular analysis was also performed and the different regions of the aorta that were analyzed are shown in FIG. 15.

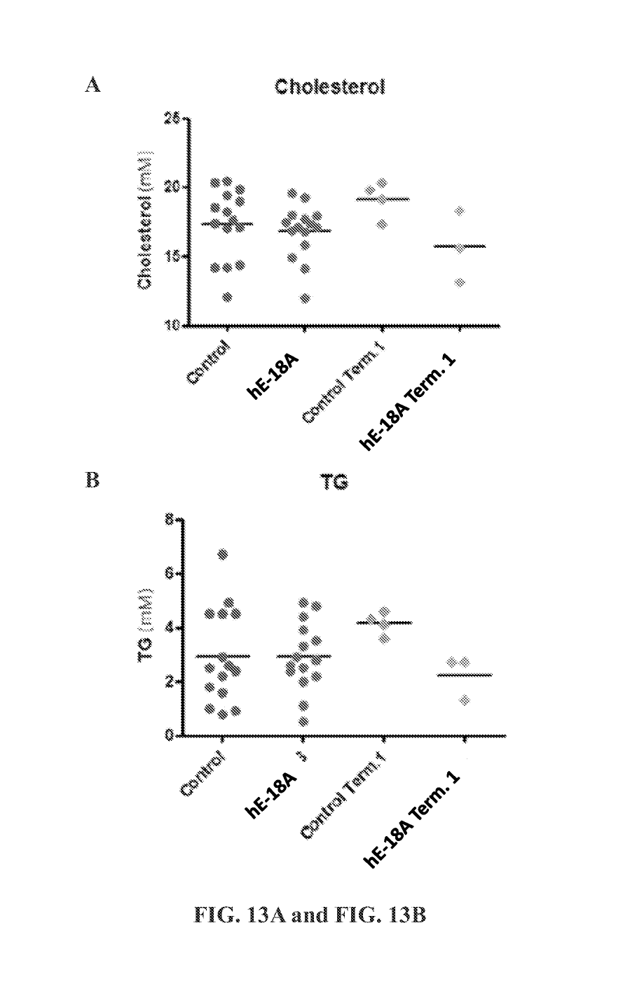

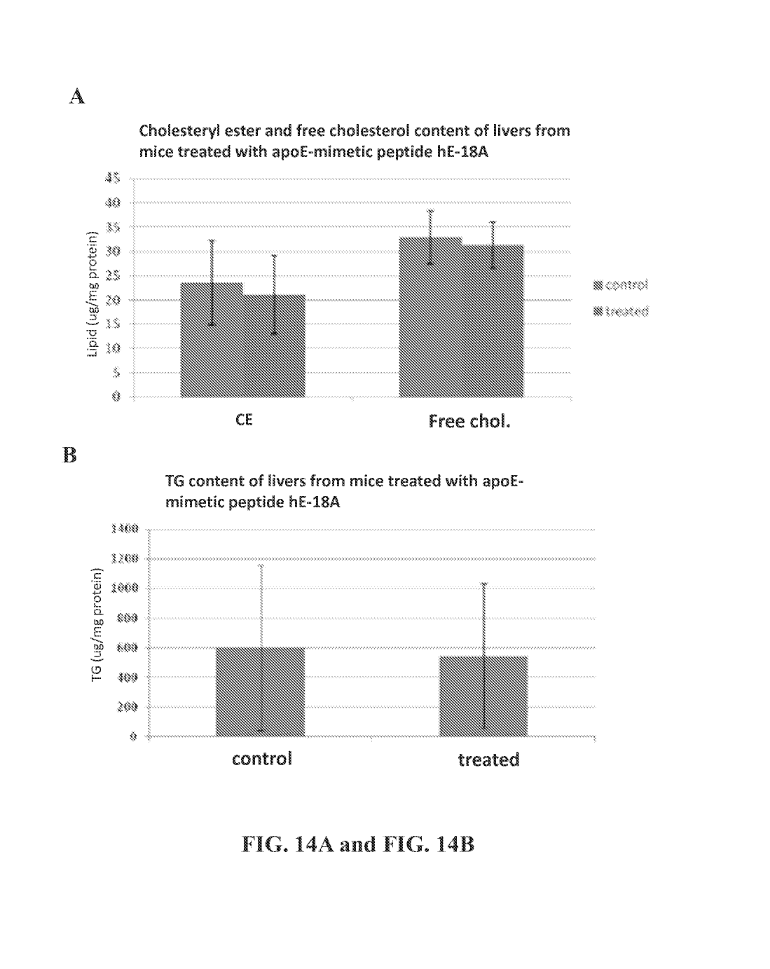

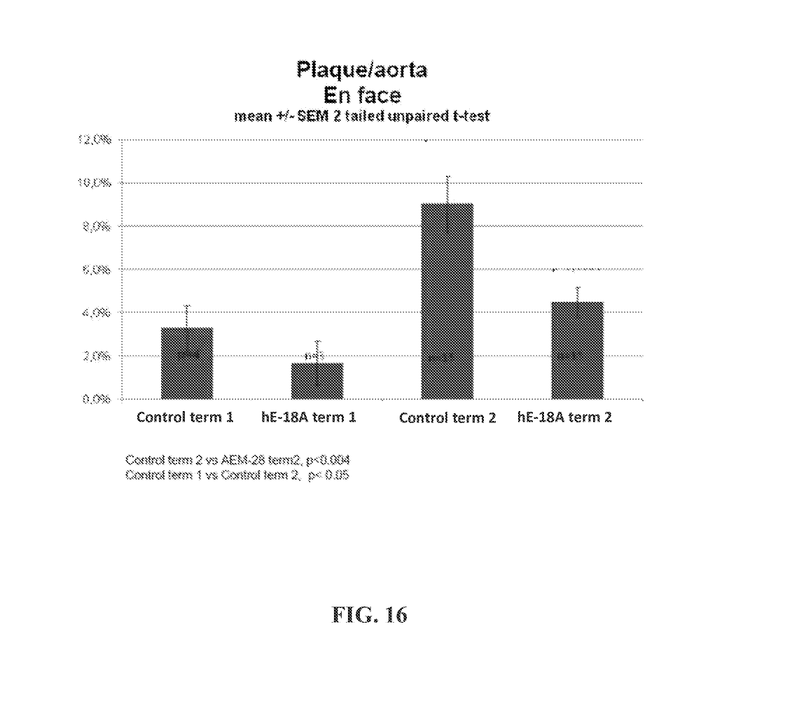

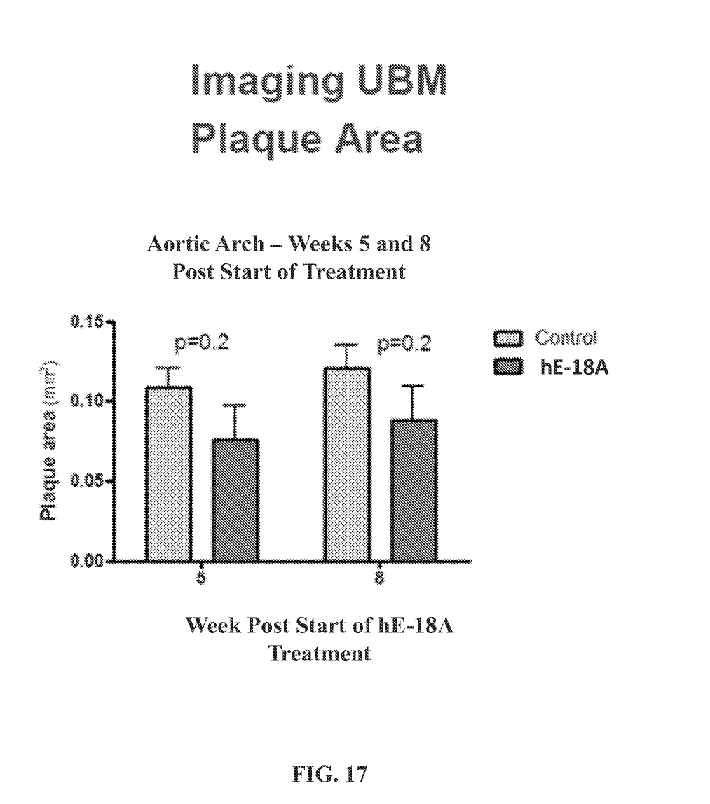

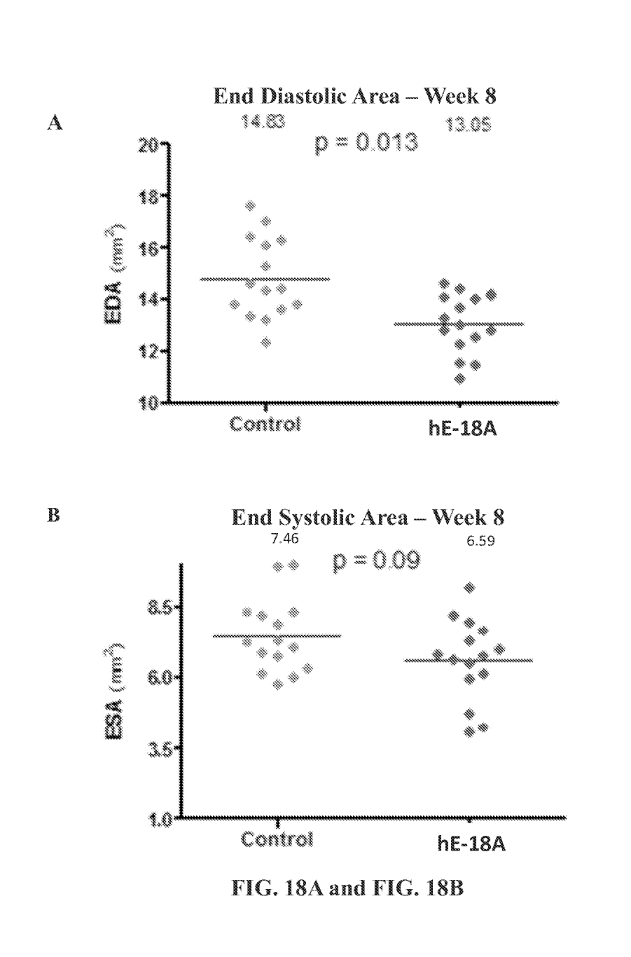

Treatment with the Apo E mimetic lowered lipid levels during the treatment period but did not appear to have sustained effects (FIGS. 13 and 14). However, the cardiovascular analysis showed that the percentage of plaques in the aorta was significantly decreased even four weeks after treatment with Apo E mimetic was withdrawn (FIGS. 16 and 17). Furthermore, both end-diastolic and end-systolic cross sectional areas of the heart were decreased four weeks after treatment with Apo E mimetics were withdrawn (FIGS. 18A and 18B). Ejection fraction remained the same. There was no increase in heart rate of ventricle wall thickness, indicating that the hearts were operating with a greater cardiac reserve to deliver the same amount of blood.

### SUMMARY

Both studies showed similar results. Although the animals had cholesterol levels of about 650 mg/dl four weeks after cessation of treatment, the animals treated with 5 mg/kg of AchE18ANH2 had decreased atherosclerotic lesions in the thoracic aorta by about 50%, and reduced lesions in the aortic arch by about 35% at the end of treatment.

Four weeks after the cessation of treatment, the end diastolic and end systolic areas were reduced in the treated animals. Ejection fraction and heart rate was unchanged. As a result, cardiac output was maintained at a lower heart volume, increasing cardiac reserve and efficiency. This is indicative of decreased arterial stiffness and enhanced vasorelaxation.

Four weeks after cessation of treatment, PON-1 activity remained elevated in the Apo E mimetic treated animals. PON-1 is a major anti-atherosclerotic component of HDL, and thus improves HDL function. All of these sustained benefits indicate that a course of treatment with Ac-hE18A-NH2 reduces atherosclerotic burden, improves the health of the artery wall, and rejuvenates the artery wall, regressing atherosclerosis caused by chronic hypercholesterolemia and inflammation.

1. A dosing regimen comprising at least one treatment cycle followed by a rest phase, wherein the treatment cycle comprises administering an effective amount of an Apo E mimetic to allow for a sustained therapeutic effect after withdrawal of the Apo E mimetic, wherein the Apo E mimetic is not administered during the rest phase.

2. The dosing regimen of claim 1, wherein the treatment cycle comprises administration of an effective amount of the Apo E mimetic once a week for three months.

3. The dosing regimen of claim 1, wherein the treatment cycle comprises administration of an effective amount of the Apo E mimetic once every two weeks for up to 12 weeks.

4. The dosing regimen of claim 1, wherein the dosing regimen further comprises a second treatment cycle after the rest phase.

5. A method of treating atherosclerosis comprising administering to a subject an effective amount of an Apo E mimetic for at least one treatment cycle, wherein the treatment cycle comprises administering an effective amount of an Apo E mimetic to allow for a sustained therapeutic effect after withdrawal of the Apo E mimetic, wherein the treatment cycle is followed by a rest phase, wherein Apo E mimetic is not administered during the rest phase.

6. The method of claim 5, wherein the rest phase is at least four weeks.

7. The method of claim 5, further comprising a second treatment cycle after the rest phase.

8. The method of claim 7, wherein the second treatment cycle is administered after a four week rest phase.

9. The method of claim 7, wherein the second treatment cycle is administered one year from the beginning of the initial treatment cycle.

10. The method of claim 5, wherein an atherosclerosis therapeutic other than an Apo E mimetic is administered during the rest phase.

11. The method of claim 10, wherein the atherosclerosis therapeutic other than an Apo E mimetic is a conventional lipid lowering therapy.

12. The method of claim 11, wherein the conventional lipid lowering therapy is a statin.

13. The method of claim 5, wherein the Apo E mimetic is LRKLRKRLLRDWLKAFYDKVAEKLKEAF.

14. The method of claim 5, wherein the treatment cycle comprises administration of an effective amount of an Apo E mimetic once a week for three months.

15. A dosing regimen comprising at least one treatment cycle followed by a rest phase, wherein the treatment cycle comprises administering an effective amount of an Apo E mimetic to allow for a sustained therapeutic effect after withdrawal of the Apo E mimetic, wherein the Apo E mimetic consists of the amino acid sequence LRKLRKRLLRDWLKAFYDKVAEKLKEAF (SEQ ID NO: 1), wherein the Apo E mimetic is not administered during the rest phase.

16. The dosing regimen of claim 15, wherein the amino acid sequence LRKLRKRLLRDWLKAFYDKVAEKLKEAF (SEQ ID NO: 1) is end protected with an acetyl group protecting the amino terminus and an amide group protecting the carboxyl terminus.

17. (canceled)
